(19) United States
(12) Patent Application Publication
(10) Pub. No.: US 2010/0215579 A1
(43) Pub. Date: Aug. 26, 2010

(54) PHEN-NAPHTHALENE AND PHEN-QUINOLINE DERIVATIVES AND THEIR USE FOR BINDING AND IMAGING AMYLOID PLAQUES

(75) Inventors: Hank F. Kung, Wynnewood, PA (US); Mei-Ping Kung, Wynnewood, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia (PA)

(21) Appl. No.: 12/595,111

(22) PCT Filed: Apr. 10, 2008
(86) PCT No.: PCT/US08/59864

§ 371 Date: Mar. 11, 2010

Related U.S. Application Data
(60) Provisional application No. 60/907,598, filed on Apr. 10, 2007.

(51) Int. Cl. A61K 51/04; C07C 43/225; C07D 215/227; C07C 211/44; A61K 31/085; A61K 31/47; A61K 31/136; A61P 25/28

(52) U.S. Cl. 424/1.89; 568/633; 546/158; 564/428; 514/721; 514/312; 514/657

(57) ABSTRACT

This invention relates to methods of imaging amyloid deposits, radiolabeled compounds, and methods of making radiolabeled compounds useful in imaging amyloid deposits. This invention also relates to compounds and methods of making compounds for inhibiting the aggregation of amyloid proteins to form amyloid deposits and methods of delivering therapeutic agents to amyloid deposits.

### CROSS-REFERENCE TO RELATED APPLICATION

This application claims the benefit of U.S. Provisional Application No. 60/907,598, filed Apr. 10, 2007, the entirety of which is incorporated herein by reference.

### STATEMENT REGARDING FEDERALLY-SPONSORED RESEARCH

Part of the work performed during development of this invention utilized U.S. Government funds. The U.S. Government has certain rights in this invention under grant number AG-022559 awarded by the National Institutes of Health.

### FIELD OF THE INVENTION

This invention relates to novel bioactive compounds, methods of diagnostic imaging using radiolabeled compounds, and methods of making radiolabeled compounds.

### BACKGROUND OF THE INVENTION

Alzheimer's disease (AD) is a progressive neurodegenerative disorder characterized by cognitive decline, irreversible memory loss, disorientation, and language impairment. Alzheimer's disease (AD) is a common neurodegenerative disease of the brain. It is a significant medical problem with a high prevalence in millions of elder people. Major neuropathology observations of postmortem AD brains depict the presence of senile plaques (containing β-amyloid (Aβ) aggregates) and neurofibrillary tangles (highly phosphorylated tau proteins). Currently, there is no definitive imaging method to diagnose AD, except by postmortem biopsy and staining of the brain tissue which demonstrates the senile plaques containing predominantly AB aggregates.

Several genomic factors have been linked to AD. Familial AD (or early onset AD) has been reported to have mutations in genes encoding β-amyloid precursor protein (APP), presenilin 1 (PS1) and presenilin 2 (PS2) (Berezovska, O, A Lleo, L D Herl, et al. “Familial Alzheimer's disease presenilin 1 mutations cause alterations in the conformation of presenilin and interactions with amyloid precursor protein.” J Neurosci 25:3009 (2005); Deng, Y, L Tarassishin, V Kallhoff, et al. “Deletion of presenilin 1 hydrophilic loop sequence leads to impaired gamma-secretase activity and exacerbated amyloid pathology.” J Neurosci 26:3845 (2006); Hardy, J, D J Selkoe “The amyloid hypothesis of Alzheimer's disease: progress and problems on the road to therapeutics.” Science 297:353 (2002); Selkoe, D J “Alzheimer's disease: genes, proteins, and therapy.” Physiol Rev 81:741 (2001)). The exact mechanisms of these mutations which lead to the development of AD, are not fully understood; however, the formation of Aβ plaques in the brain is a pivotal event in the pathology of Alzheimer's disease.

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins. Formation of soluble and diffusible Aβ and Aβ aggregates in the brain are now considered the critical events, which produce various toxic effects in neuronal cells leading to the formation of neuritic plaques (Catalano, S M, E C Dodson, D A Henze, et al. “The Role of Amyloid-Beta Derived Diffusible Ligands (ADDLs) in Alzheimer's Disease.” Curr Top Med Chem 6:597 (2006); Hardy, (2002); Jicha, G A, J E Parisi, D W Dickson, et al. “Neuropathologic outcome of mild cognitive impairment following progression to clinical dementia.” Arch Neurol 63:674 (2006); Rosenberg, R N “Explaining the cause of the amyloid burden in Alzheimer disease.” Arch Neurol 59:1367 (2002); Thal, D R, E Capetillo-Zarate, K Del Tredici, et al. “The development of amyloid beta protein deposits in the aged brain.” Sci Aging Knowledge Environ 2006:re1, (2006)). Recent reports have suggested that β-amyloid aggregates, i.e. Aβ plaques, in the brain play a key role in a cascade of events leading to AD. Postmortem examination of AD brain sections reveals abundant senile plaques (SPs) composed of amyloid-β (Aβ) peptides and numerous neurofibrillary tangles (NFTs) formed by filaments of highly phosphorylated tau proteins (for recent reviews and additional citations see Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

While the exact mechanisms underlying AD are not fully understood, all pathogenic familial AD (FAD) mutations studied thus far increase production of the more amyloidogenic 42-43 amino-acid long form of the Aβ peptide. Thus, at least in FAD, dysregulation of Aβ production appears to be sufficient to induce a cascade of events leading to neurodegeneration. Indeed, the amyloid cascade hypothesis suggests that formation of extracellular fibrillar Aβ aggregates in the brain may be a pivotal event in AD pathogenesis (Selkoe, D. J., “Biology of β-amyloid Precursor Protein and the Mechanism of Alzheimer's Disease,” Alzheimer's Disease, Lippincot Williams & Wilkins, Philadelphia, Pa. (1999), pp. 293-310; Selkoe, D. J., J. Am. Med. Assoc. 283:1615-1617 (2000); Naslund, J., et al., J. Am. Med. Assoc. 283:1571-1577, (2000); Golde, T. E., et al., Biochimica et Biophysica Acta 1502:172-187 (2000)).

Significant circumstantial evidence suggests that fibrillary Aβ plaques consisting predominately of aggregates of Aβ₄₀ and Aβ₄₂ peptides play a major role in AD pathogenesis -“Amyloid Cascade Hypothesis” (Armstrong, R A “Plaques and tangles and the pathogenesis of Alzheimer's disease.” Folia Neuropathol 44:1 (2006); Golde, T E “The Abeta hypothesis: leading us to rationally-designed therapeutic strategies for the treatment or prevention of Alzheimer disease.” Brain Pathol 15:84 (2005); Hardy, J “Has the amyloid cascade hypothesis for Alzheimer's disease been proved?” Curr Alzheimer Res 3:71 (2006); Hardy (2002); Marchesi, V T “An alternative interpretation of the amyloid Abeta hypothesis with regard to the pathogenesis of Alzheimer's disease.” Proc Natl Acad Sci USA 102:9093 (2005)). ApoE4 expression appears to increase the risk of AD (Fryer, J D, J W Taylor, R B DeMattos, et al. “Apolipoprotein E markedly facilitates age-dependent cerebral amyloid angiopathy and spontaneous hemorrhage in amyloid precursor protein transgenic mice.” J Neurosci 23:7889 (2003)). It is likely that amyloid precursor protein (APP) is degraded by several proteases, among which the catabolism reactions of β- and γ-secretases on APP lead to the production of excess Aβ. The excessive burden of Aβ, produced by various normal or abnormal mechanisms, may represent the starting point of neurodegenerative events. The fibrillar aggregates of amyloid peptides, Aβ₄₀ and Aβ₄₂, are major metabolic peptides derived from amyloid precursor protein found in senile plaques and cerebrovascular amyloid deposits in AD patients (Xia, W., et al., J. Proc. Natl. Acad. Sci. U.S.A. 97:9299-9304, (2000)). Prevention and reversal of Aβ plaque formation are being targeted as a treatment for this disease (Selkoe, D., J. JAMA 283:1615-1617 (2000); Wolfe, M. S., et al., J. Med. Chem. 41:6-9, 1998; Skovronsky, D. M., and Lee, V. M., Trends Pharmacol. Sci. 21:161-163 (2000)).

Early appraisal of clinical symptoms for diagnosis of AD is often difficult and unreliable (Boss, M A “Diagnostic approaches to Alzheimer's disease.” Biochim Biophys Acta 1502:188 (2000)). Positron emission tomography (PET) and single photon emission tomography (SPECT) imaging of regional cerebral blood flow (rCBF) for diagnosis and monitoring of patients with AD have been reported (Ishii, K, S Minoshima “PET is better than perfusion SPECT for early diagnosis of Alzheimer's disease—for.” Eur J Nucl Med Mol Imaging 32:1463 (2005); Mega, M S, ID Dinov, L Lee, et al. “Orbital and dorsolateral frontal perfusion defect associated with behavioral response to cholinesterase inhibitor therapy in Alzheimer's disease.” J Neuropsychiatry Clin Neurosci 12:209 (2000a); Mega, M S, L Lee, I D Dinov, et al. “Cerebral correlates of psychotic symptoms in Alzheimer's disease.” J Neurol Neurosurg Psychiatry 69:167 (2000b); Tang, B N, S Minoshima, J George, et al. “Diagnosis of suspected Alzheimer's disease is improved by automated analysis of regional cerebral blood flow.” Eur J Nucl Med Mol Imaging 31:1487 (2004)). Diagnosis of AD based on regional glucose metabolism in the brain has been evaluated using PET imaging with [¹⁸F]2-fluoro-2-deoxyglucose (FDG). The overall performance of FDG/PET is favorable for routine clinical evaluation of suspected AD (Frey, K A, S Minoshima, D E Kuhl “Neurochemical imaging of Alzheimer's disease and other degenerative Dementias.” Q J Nucl Med 42:166 (1998); Hoffman, J M, K A Welsh-Bohmer, M Hanson, et al. “FDG PET imaging in patients with pathologically verified d41ementiaj.” J Nucl Med 41:1920 (2000); Minoshima, S “Imaging Alzheimer's disease: clinical applications.” Neuroimaging Clin N Am 13:769 (2003); Minoshima, S, B Giordani, S Berent, et al. “Metabolic reduction in the posterior cingulate cortex in very early Alzheimer's disease.” Ann Neurol 42:85 (1997); Phelps, M E “PET: the merging of biology and imaging into molecular imaging.” J Nucl Med 41:661 (2000); Silverman, D H S, M E Phelps “Invited Commentary: Evaluating Dementia Using PET: How Do We Put into Clinical Perspective What We Know to Date?” J Nucl Med 41:1929 (2000)). While imaging rCBF and glucose metabolism may have some use in AD patients, none of these modalities provide any information on the presence or quantity of Aβ aggregates in the brain.

Various approaches in trying to inhibit the production and reduce the accumulation of fibrillar Aβ in the brain are currently being evaluated as potential therapies for AD (Skovronsky, D. M. and Lee, V. M., Trends Pharmacol. Sci. 21:161-163 (2000); Vassar, R., et al., Science 286:735-741, 1999; Wolfe, M. S., et al., J. Med. Chem. 41:6-9, 1998; Moore, C. L., et al., J. Med. Chem. 43:3434-3442 (2000); Findeis, M. A., Biochimica et Biophysica Acta 1502:76-84, 2000; Kuner, P., Bohrmann, et al., J. Biol. Chem. 275:1673-1678 (2000)). It is therefore of interest to develop ligands that specifically bind fibrillar Aβ aggregates. Since extracellular SPs are accessible targets, these new ligands could be used as in vivo diagnostic tools and as probes to visualize the progressive deposition of Aβ in studies of AD amyloidogenesis in living patients. Development of Aβ plaque-specific imaging agents has been reported previously (for review see Blennow, K, H Zetterberg “Pinpointing plaques with PIB.” Nat Med 12:753 (2006b); Huddleston, D E, S A Small “Technology Insight: imaging amyloid plaques in the living brain with positron emission tomography and MRI.” Nat Clin Pract Neurol 1:96 (2005); Mathis, C A, Y Wang, W E Klunk “Imaging β-amyloid plaques and neurofibrillary tangles in the aging human brain.” Curr Pharm Des 10:1469 (2004); Nichols, L, V W Pike, L Cai, et al. “Imaging and in vivo quantitation of beta-amyloid: an exemplary biomarker for Alzheimer's disease?” Biol Psychiatry 59:940 (2006); Schmidt, B, H A Braun, R Narlawar “Drug development and PET-diagnostics for Alzheimer's disease.” Curr Med Chem 12:1677 (2005)).

Potential ligands for detecting Aβ aggregates in the living brain must cross the intact blood-brain barrier. Thus brain uptake can be improved by using ligands with relatively smaller molecular size and increased lipophilicity. Highly conjugated thioflavins (S and T) are commonly used as dyes for staining the Aβ aggregates in the AD brain (Elhaddaoui, A., et al., Biospectroscopy 1:351-356 (1995)). To this end, several interesting approaches for developing fibrillar Aβ aggregate-specific ligands have been reported (Ashburn, T. T., et al., Chem. Biol. 3:351-358 (1996); Han, G., et al., J. Am. Chem. Soc. 118:4506-4507 (1996); Klunk, W. E., et al., Biol. Psychiatry 35:627 (1994); Klunk, W. E., et al., Neurobiol. Aging 16:541-548 (1995); Klunk, W. E., et al., Society for Neuroscience Abstract 23:1638 (1997); Mathis, C. A., et al., Proc. XIIth Intl. Symp. Radiopharm. Chem., Uppsala, Sweden: 94-95 (1997); Lorenzo, A. and Yankner, B. A., Proc. Natl. Acad. Sci. U.S.A. 91:12243-12247 (1994); Zhen, W., et al., J. Med. Chem. 42:2805-2815 (1999); Klunk, W. E., et al., J. Histochem. Cytochem. 37:1273-1281 (1989)).

The approach has been based on highly conjugated dyes, such as Congo Red and Chrysamine G (CG) (Dezutter, N A, R J Dom, T J de Groot, et al. “^{99m}Tc-MAMA-chrysamine G, a probe for beta-amyloid protein of Alzheimer's disease.” Eur J Nucl Med 26:1392 (1999); Klunk, W E, M L Debnath, A M Koros, et al. “Chrysamine-G, a lipophilic analogue of Congo red, inhibits Aβ-induced toxicity in PC12 cells.” Life Sci 63:1807 (1998); Klunk, W E, M L Debnath, J W Pettegrew “Small-molecule beta-amyloid probes which distinguish homogenates of Alzheimer's and control brains.” Biol Psychiatry 35:627 (1994)). Thioflavin S and T have also been used in fluorescent staining of plaques and tangles in postmortem AD brain sections (Elhaddaoui, A, E Pigorsch, A Delacourte, et al. “Competition of congo red and thioflavin S binding to amyloid sites in Alzheimer's diseased tissue.” Biospectroscopy 1:351 (1995)). More abbreviated forms of Chrysamine G (CG), such as styrylbenzenes, have been reported as fluorescent dyes for staining amyloid aggregates (Link, C D, C J Johnson, V Fonte, et al. “Visualization of fibrillar amyloid deposits in living, transgenic Caenorhabditis elegans animals using the sensitive amyloid dye, X-34.” Neurobiol Aging 22:217 (2001); Styren, S D, R L Hamilton, G C Styren, et al. “X-34, a fluorescent derivative of Congo Red: a novel histochemical stain for Alzheimer's disease pathology.” J Histochem Cytochem 48:1223 (2000)). They are useful research tools but these charged and bulky agents do not cross intact blood-brain barrier.

A highly lipophilic tracer, [¹⁸F]FDDNP, for binding both tangles (mainly composed of hyperphosphorylated tau protein) and plaques (containing Aβ protein aggregates) has been reported. (Shoghi-Jadid K, et al., Am J Geriatr Psychiatry. 10:24-35 (2002); Barrio, J R, S-C Huang, G Cole, et al. “PET imaging of tangles and plaques in Alzheimer's disease with a highly hydrophobic probe.” J Lab Compds Radiopharm 42 Suppl. 1:S194, (1999a); Barrio, J R, S C Huang, G M Cole, et al. “PET imaging of tangles and plaques in Alzheimer's disease.” J Nucl Med 40:70 P, (1999b)). Preliminary studies in humans suggested that [¹⁸F]FDDNP showed a higher retention in regions of brain suspected of having tangles and plaques (Kepe, V, J R Barrio, S C Huang, et al. “Serotonin 1A receptors in the living brain of Alzheimer's disease patients.” Proc Natl Acad Sci USA 103:702 (2006); Shoghi-Jadid, K, J R Barrio, V Kepe, et al. “Exploring a mathematical model for the kinetics of beta-amyloid molecular imaging probes through a critical analysis of plaque pathology.” Mol Imaging Biol 8:151 (2006); Shoghi-Jadid, K, J R Barrio, V Kepe, et al. “Imaging beta-amyloid fibrils in Alzheimer's disease: a critical analysis through simulation of amyloid fibril polymerization.” Nucl Med Biol 32:337 (2005); Shoghi-Jadid, K, G W Small, E D Agdeppa, et al. “Localization of neurofibrillary tangles and beta-amyloid plaques in the brains of living patients with Alzheimer disease: Binding characteristics of radiofluorinated 6-dialkylamino-2-naphthylethylidene derivatives as positron emission tomography imaging probes for beta-amyloid plaques in Alzheimer disease.” Am J Geriatr Psychiatry 10:24, (2002)). Using positron-emission tomography (PET), it was reported that this tracer specifically labeled deposits of plaques and tangles in nine AD patients and seven comparison subjects. (Nordberg A. Lancet Neurol. 3:519-27 (2004)). Using a novel pharmacokinetic analysis procedure called the relative residence time of the brain region of interest versus the pons, differences between AD patients and comparison subjects were demonstrated. The relative residence time was significantly higher in AD patients. This is further complicated by an intriguing finding that FDDNP competes with some NSAIDs for binding to Aβ fibrils in vitro and to Aβ plaques ex vivo (Agdeppa E D, et al. 2001; Agdeppa E D, et al., Neuroscience. 2003; 117:723-30).

A neutral and lipophilic thioflavin derivative, [¹¹C]6-OH-BTA-1 (PIB), showed excellent brain penetration and initial brain uptake, and displayed a high binding affinity to Aβ plaques (Kᵢ=2.8 nM) (Klunk, W E, Y Wang, G-f Huang, et al. “Uncharged thioflavin-T derivatives bind to amyloid-beta protein with high affinity and readily enter the brain.” Life Sci 69:1471 (2001); Mathis, C A, B J Bacskai, STBMC Kajdasz, et al. “A lipophilic thioflavin-T derivative for positron emission tomography (PET) imaging of amyloid in brain.” Bioorg Med Chem Lett 12:295 (2002a); Mathis, C A, Y Wang, W E Klunk “Imaging b-amyloid plaques and neurofibrillary tangles in the aging human brain.” Curr Pharm Des 10:1469 (2004); (Mathis C A, et al., Curr Pharm Des. 10:1469-92 (2004); Mathis C A, et al., Arch. Neurol. 62:196-200 (2005)). Contrary to that observed for [¹⁸F]FDDNP, [¹¹C]6-OH-BTA-1 binds specifically to fibrillar Aβ in vivo. Patients with diagnosed mild AD showed marked retention of [¹¹C]6-OH-BTA-1 in the cortex, known to contain large amounts of amyloid deposits in AD. In the AD patient group, [¹¹C]6-OH-BTA-1 retention was increased most prominently in the frontal cortex. Large increases also were observed in parietal, temporal, and occipital cortices and in the striatum. [¹¹C]6-OH-BTA-1 retention was equivalent in AD patients and comparison subjects in areas known to be relatively unaffected by amyloid deposition (such as subcortical white matter, pons, and cerebellum). Fluorinated PIB and related neutral thioflavin derivatives, such as BTA-1, have also been reported (Mathis, C A, DP Holt, Y Wang, et al. “¹⁸F-labeled thioflavin-T analogs for amyloid assessment.” J Nucl Med 43:166 P, (2002b)).

In the past few years, successful PET imaging study in AD patients with [¹¹C]PIB has been reported (Klunk, W E, B J Lopresti, M D Ikonomovic, et al. “Binding of the positron emission tomography tracer Pittsburgh compound-B reflects the amount of amyloid-beta in Alzheimer's disease brain but not in transgenic mouse brain.” J Neurosci 25:10598, (2005); Lopresti, B J, W E Klunk, C A Mathis, et al. “Simplified Quantification of Pittsburgh Compound B Amyloid Imaging PET Studies: A Comparative Analysis.” J Nucl Med 46:1959 (2005); Mathis, C A, W E Klunk, J C Price, et al. “Imaging technology for neurodegenerative diseases: progress toward detection of specific pathologies.” Arch Neurol 62:196 (2005); Price, J C, W E Klunk, B J Lopresti, et al. “Kinetic modeling of amyloid binding in humans using PET imaging and Pittsburgh Compound-B.” J Cereb Blood Flow Metab 25:1528 (2005)). Recently, [¹¹C]PIB has been used in testing a limited number of patients with mild cognitive impairment (MCI) (Buckner, R L, A Z Snyder, B J Shannon, et al. “Molecular, structural, and functional characterization of Alzheimer's disease: evidence for a relationship between default activity, amyloid, and memory.” J Neurosci 25:7709 (2005); Nordberg, A “PET imaging of amyloid in Alzheimer's disease.” Lancet Neurol 3:519 (2004); Price, J C, W E Klunk, B J Lopresti, et al. “Kinetic modeling of amyloid binding in humans using PET imaging and Pittsburgh Compound-B.” J Cereb Blood Flow Metab 25:1528, (2005)). Using PIB/PET to study the relationship between Aβ plaque burden and AD neurological measurements, the results seem to suggest that there are some MCI cases that convert to AD, while those with lower PIB uptake in the cortex appear to have less propensity to convert to AD (Engler, H, A Forsberg, O Almkvist, et al. “Two-year follow-up of amyloid deposition in patients with Alzheimer's disease.” Brain (2006); Mintun, M A, G N Larossa, Y I Sheline, et al. “[¹¹C]PIB in a nondemented population: potential antecedent marker of Alzheimer disease.” Neurology 67:446 (2006); Price, J C, S K Ziolko, L A Weissfeld, et al. “[O-15] Water and PIB PET imaging in Alzheimer's disease and mild cognitive impairment.” J Nucl Med:75p (abstract) (2006); Rentz, D M, J A Becker, E Moran, et al. “Amyloid imaging in AD, MCI, and highly intelligent older adults with Pittsburgh Compound-B (PIB).” J Nucl Med:289p (abstract) (2006); Villemagne, V L, S Ng, S J Gong, et al. “¹¹C-PIB PET imaging in the differential diagnosis of dementia.” J Nucl Med:74p (abstract), (2006)).

Recently, another ¹¹C labeled Aβ plaque-targeting probe, a stilbene derivative, [¹¹C]SB-13, has been studied. In vitro binding using the [³H]SB-13 suggests that the compound showed excellent binding affinity and binding can be clearly measured in the cortical gray matter, but not in the white matter of AD cases. (Kung M-P, et al., Brain Res. 1025:98-105 (2004). There was a very low specific binding in cortical tissue homogenates of control brains. The K_{d} values of [³H]SB-13 in AD cortical homogenates were 2.4±0.2 nM. High binding capacity and comparable values were observed (14-45 μmol/mg protein) (Id.). As expected, in AD patients [¹¹C]SB-13 displayed a high accumulation in the frontal cortex (presumably an area containing a high density of Aβ plaques) in mild to moderate AD patients, but not in age-matched control subjects. (Verhoeff N P, et al., Am J Geriatr Psychiatry. 12:584-95, (2004)).

Recently, there have been reports on using an in vivo multiphoton optical imaging technique for invasive imaging of senile plaques in transgenic mice (by opening the skull) (Bacskai, B J, S T Kajdasz, R H Christie, et al. “Imaging of amyloid-beta deposits in brains of living mice permits direct observation of clearance of plaques with immunotherapy.” Nat Med 7:369, (2001)). Additional improvements on developing near-infrared optical imaging agents have been reported (Bacskai, B J, G A Hickey, J Skoch, et al. “Four-dimensional multiphoton imaging of brain entry, amyloid binding, and clearance of an amyloid-beta ligand in transgenic mice.” Proc Natl Acad Sci USA 100:12462 (2003); Hintersteiner, M, A Enz, P Frey, et al. “In vivo detection of amyloid-beta deposits by near-infrared imaging using an oxazine-derivative probe.” Nat Biotechnol 23:577 (2005); Nesterov, E E, J Skoch, B T Hyman, et al. “In vivo optical imaging of amyloid aggregates in brain: design of fluorescent markers.” Angew Chem Int Ed Engl 44:5452 (2005)).

There are several potential benefits of imaging Aβ aggregates in the brain. The imaging technique will improve diagnosis by identifying potential patients with excess Aβ plaques in the brain; therefore, they may be likely to develop Alzheimer's disease. It will also be useful to monitor the progression of the disease. When anti-plaque drug treatments become available, imaging Aβ plaques in the brain may provide an essential tool for monitoring treatment. Thus, a simple, noninvasive method for detecting and quantitating amyloid deposits in a patient has been eagerly sought. Presently, detection of amyloid deposits involves histological analysis of biopsy or autopsy materials. Both methods have drawbacks. For example, an autopsy can only be used for a postmortem diagnosis.

In addition to the role of amyloid deposits in Alzheimer's disease, the presence of amyloid deposits has been shown in diseases such as Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

The direct imaging of amyloid deposits in vivo is difficult, as the deposits have many of the same physical properties (e.g., density and water content) as normal tissues. Attempts to image amyloid deposits using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have provided for poor imaging of tissue interiors.

It would be useful to have a noninvasive technique for imaging and quantitating amyloid deposits in a patient. In addition, it would be useful to have compounds that inhibit the aggregation of amyloid proteins to form amyloid deposits and a method for determining a compound's ability to inhibit amyloid protein aggregation.

### SUMMARY OF THE INVENTION

The present invention provides novel compounds of Formulas I and II. The present invention also provides diagnostic compositions comprising radiolabeled compounds of Formulas I and II and pharmaceutically acceptable carriers and/or diluents.

The invention further provides methods of imaging amyloid deposits, the methods comprising introducing into a mammal a detectable quantity of a labeled compound of Formula I or II or a pharmaceutically acceptable salt, ester, amide or prodrug thereof.

The present invention also provides methods for inhibiting the aggregation of amyloid proteins, the methods comprising administering to a mammal an amyloid inhibiting amount of a compound Formula I or II or a pharmaceutically acceptable salt, ester, amide, or prodrug thereof.

A further aspect of this invention is directed to methods and intermediates useful for synthesizing the amyloid inhibiting and imaging compounds of Formulas I and II.

### BRIEF DESCRIPTION OF THE DRAWINGS

FIG. 1 depicts Kᵢ binding data of preferred embodiments of the present invention.

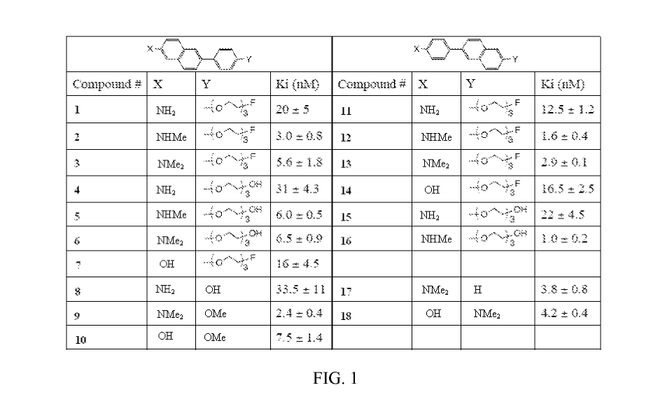

### DETAILED DESCRIPTION OF ILLUSTRATIVE EMBODIMENTS

In a first aspect the present invention is directed to compounds of Formula I:

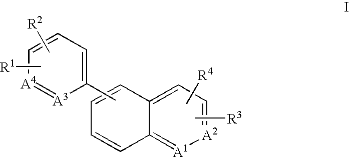

or a pharmaceutically acceptable salt or prodrug thereof, wherein:

A¹, A², A³ and A⁴ are independently C, CH, or N;

R¹ and R⁴ are each independently:
NR′R″, wherein R′ and R″ are independently hydrogen, C₁₋₄ alkyl, hydroxy(C₁₋₄)alkyl or halo(C₁₋₄)alkyl;
hydroxy;
C₁₋₄ alkoxy;
hydroxy(C₁₋₄)alkyl;
halogen;
cyano;
hydrogen;
nitro;
(C₁-C₄)alkyl;
halo(C₁-C₄)alkyl;
formyl;
O—CO(C₁₋₄ alkyl);
—COO(C₁₋₄ alkyl);
—NHCO(C₁₋₄ alkyl), or
radiohalogen;

R² and R³ are hydrogen or fragments i, ii or iii, wherein:
fragment i is:

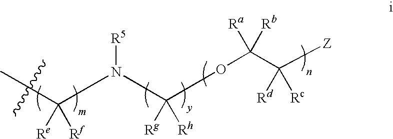

wherein, n is an integer from 1 to 10; m is an integer from 0 to 5; y is an integer from 1 to 5; R⁵ is hydrogen, C₁₋₄ alkyl, or hydroxy(C₁₋₄)alkyl; R^{a}, R^{b}, R^{c}, R^{d}, R^{e}, R^{f}, R^{g} and R^{h} are each independently hydrogen, halogen, hydroxy, C₁₋₄ alkoxy, C₁₋₄ alkyl or hydroxy(C₁₋₄)alkyl; and Z is:
   a) X, wherein X is hydrogen, hydroxy, halogen, radiohalogen, C₁₋₄ alkoxy, hydroxy(C₁₋₄alkyl, halo(C₁₋₄)alkyl, radiohalo(C₁₋₄)alkyl or NR^{x}R^{y}, wherein R^{x} and R^{y} are independently hydrogen, C₁₋₄ alkyl, hydroxy(C₁₋₄alkyl, radiohalo(C₁₋₄)alkyl or halo(C₁₋₄)alkyl;
   b) benzoyloxy, phenyl(C₁₋₄)alkyl, aryloxy or C₆₋₁₀ aryl, each of which is substituted by X; or
   c) Zc, wherein Zc is:

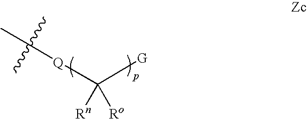

wherein, p is an integer from 1 to 4, Q is O or NR⁵; G is —C═C—(R^{G})X or —C≡C—X, wherein R^{G} is hydrogen or (C₁₋₄)alkyl; Rⁿ and R^{o} are independently hydrogen, hydroxy or (C₁₋₄)alkyl, and X and R⁵ are as described above;
fragment ii is:

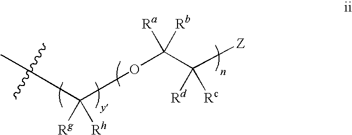

wherein, y′ is an integer from 0 to 5, preferably 0 to 3, most preferably 0 or 1; and
   n, R^{a}, R^{b}, R^{c}, R^{d}, R^{g}, R^{h} and Z are as described above; and and fragment iii is:

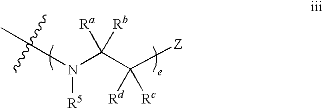

wherein, e is 0 or 1, and Z, R^{a}, R^{b}, R^{c}, R^{d} and R⁵ are as described above;

R⁴ is hydrogen, hydroxy, halogen, radiohalogen, (C₁₋₄)alkyl, (C₁₋₄)alkoxy, hydroxy(C₁₋₄alkyl or NR′R″, wherein R′ and R″ are independently hydrogen, (C₁₋₄)alkyl, hydroxy(C₁₋₄alkyl or halo(C₁₋₄)alkyl; provided that, X is F or ¹⁸F or contains F or ¹⁸F, preferably ¹⁸F; or one of R¹ and R⁴ is F, ¹⁸F, Br, ⁷⁶Br, ⁷⁷Br, I, ¹²³I, ¹²⁵I and ¹³¹I; or one of R² and R³ is other than hydrogen.

Preferred values of R¹ include hydroxy, (C₁₋₄)alkoxy, —NHCO(C₁₋₄ alkyl), —O—CO(C₁₋₄ alkyl), —COO(C₁₋₄ alkyl) and NR′R″, wherein R′ and R″ are as described above. More preferably, R¹ is hydroxy or NR′R″, wherein R′ and R″ are independently hydrogen or C₁₋₄ alkyl. The more preferred value of (C₁₋₄alkyl in these embodiments is methyl. Preferably, R¹ is at the para position of the phenyl relative to the naphthalene ring.

Preferably, R⁴ is hydrogen, halogen or radiohalogen. It is preferred that if X does not contain a halogen or radiohalogen, then R⁴ is a halogen or radiohalogen. In those embodiments where X does not contain F or ¹⁸F, then R⁴ is F, ¹⁸F, I, ¹²³I, ¹²⁵I, ¹³¹I, Br, ⁷⁶Br, or ⁷⁷Br.

In the present invention, A¹, A², A³ and A⁴ are independently C, CH, or N. Preferably, one of A¹ and A² is C, and the other is C or N. When one of A¹ and A² is N, it is more preferred that A², which is in the meta position relative to the alkene bridge, is N. Preferably, both A³ and A⁴ are C. In another preferred embodiment, one of A³ and A⁴ is N. When one of A³ and A⁴ is N, it is more preferred that A⁴, which is in the meta position relative to the alkene bridge, is N. In especially preferred embodiments, A¹ is C, A² is C or N, A³ is C and A⁴ is C or N.

Each of these fragments i, ii, and iii contains a Z group. Each Z group, as shown above, contains an X moiety. The X moiety is hydrogen, hydroxy, halogen, radiohalogen, C₁₋₄ alkoxy, hydroxy(C₁₋₄alkyl, halo(C₁₋₄)alkyl, radiohalo(C₁₋₄)alkyl or NR^{x}R^{y}, wherein R^{x} and R^{y} are independently hydrogen, C₁₋₄ alkyl, hydroxy(C₁₋₄alkyl, radiohalo(C₁₋₄)alkyl or halo(C₁₋₄)alkyl. Fragments i, ii and iii are discussed more fully below.

As previously described, fragment i is:

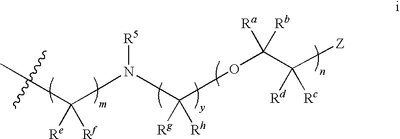

In all embodiments, n is an integer from 1 to 10. Preferably, n is an integer from 1 to 6. More preferably, n is an integer from 2 to 6, and most preferably, n is 3. In all embodiments, m is an integer from 0 to 5. Preferably, m is an integer from 0 to 3. More preferably, m is 0 or 1, and most preferably m is 0. In all embodiments, y is an integer from 1 to 5. Preferably, y is an integer from 1 to 3. More preferably, y is an integer from 1 to 2, and most preferably, y is 2. In all embodiments, R⁵ is hydrogen, (C₁₋₄)alkyl or hydroxy(C₁₋₄alkyl. More preferably, R⁵ is hydrogen or C₁₋₄ alkyl. Most preferably, R⁵ is hydrogen. In all embodiments, R^{a}, R^{b}, R^{c}, R^{d}, R^{e}, R^{f}, R^{g} and R^{h} are independent of one another and are hydrogen, halogen, hydroxy, C₁₋₄ alkoxy, (C₁₋₄)alkyl or hydroxy(C₁₋₄alkyl. Preferably, R^{a}, R^{b}, R^{c}, R^{d}, R^{e}, R^{f}, R^{g} and R^{h} are independently hydrogen, hydroxy, hydroxy(C₁₋₄alkyl or (C₁₋₄)alkyl. More preferably, R^{a}, R^{b}, R^{c}, R^{d}, R^{e}, R^{f}, R_{g} and R^{h} are independently hydrogen, hydroxy(C₁₋₄alkyl or (C₁₋₄)alkyl, and most preferably, hydroxy(C₁₋₄alkyl or hydrogen. When a hydroxy(C₁₋₄)alkyl is present, it is especially preferred that it is in the R^{c} or R^{d} position. In all embodiments, Z is: a) X, wherein X is hydrogen, halogen, radiohalogen, (C₁₋₄)alkoxy, hydroxy, hydroxy(C₁₋₄alkyl, halo(C₁₋₄)alkyl, radiohalo(C₁₋₄)alkyl or NR^{x}R^{y}, wherein R^{x} and R^{y} are as described above; b) one of the following groups, each of which contains X as a substituent: benzoyloxy, phenyl(C₁₋₄)alkyl, aryloxy, such as phenoxy, and (C₆₋₁₀)aryl; or c) Zc:

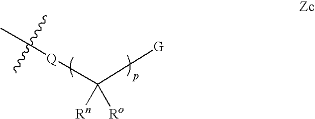

wherein, p is an integer from 1 to 4, preferably 2, Q is O or NR⁵, G is —C═C—(R^{G})X or —C≡C—X, wherein R^{G} is hydrogen or (C₁₋₄)alkyl, X and R⁵ are as described above, and Rⁿ and R^{o} are each independently hydrogen, hydroxy or (C₁₋₄)alkyl.

Structures of Formula I where A³ and A⁴ are both C and that contain fragment i include:

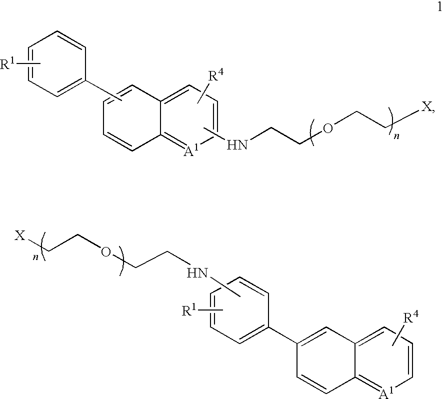

wherein, R¹, R⁴, A¹, n and X are as described above; and when Z is Zc:

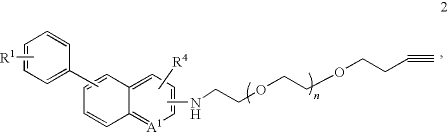

wherein, R¹, R⁴, A¹ and n are as described above. More preferably, compounds of structure 1 are those where n is an integer from 1 to 6; R¹ is hydroxy, (C₁₋₄)alkoxy, —NHCO(C₁₋₄ alkyl) or NR′R″, wherein R′ and R″ are independently hydrogen or (C₁₋₄)alkyl; R⁴ is hydrogen, (C₁₋₄)alkyl, (C₁₋₄)alkoxy, halogen or radiohalogen; and X is s hydrogen, halogen, radiohalogen, (C₁₋₄)alkoxy, hydroxy or NR^{x}R^{y}, wherein R^{x} and R^{y} are as described above; provided that X contains F or ¹⁸F, preferably ¹⁸F, or R⁴ is F, ¹⁸F, Br, ⁷⁶Br, ⁷⁷Br, I, ¹²³I, ¹²⁵I, ¹²⁵I or ¹³¹I. The most preferred compounds of structure 1 include the above proviso, and are those where n is 3; R¹ is hydroxy or —NR′R″, wherein R′ and R″ are independently hydrogen or (C₁₋₄)alkyl; R⁴ is hydrogen, halogen or radiohalogen; and X is hydroxy, halogen or radiohalogen.

As shown above, fragment ii is as follows:

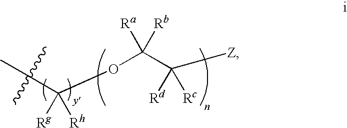

wherein, all preferred values of n, R^{a}, R^{b}, R^{c}, R^{d}, R^{g}, R^{h}, and Z are as described above. Useful values of y′ in fragment ii are integers from 0 to 5, preferably 0 to 3, and most preferably 0 or 1. Specifically, in preferred embodiments of Formula I that contain fragment ii, n is an integer from 1 to 10; y′ is an integer from 0 to 3; R^{a}, R^{b}, R^{c}, R^{d}, R^{g} and R^{h} are each independently as described above; and Z is as described above; provided that X contains F or ¹⁸F, preferably ¹⁸F, or R⁴ is F, ¹⁸F, Br, ⁷⁶Br, ⁷⁷Br, I, ¹²³I, ¹²⁵I or ¹³¹I.

Structures of Formula I where A³ and A⁴ are both C and that contain fragment ii include:

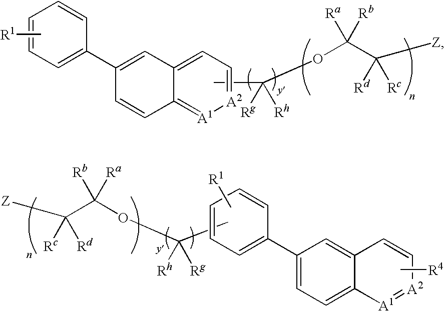

and when y′ is 0, exemplary compounds include:

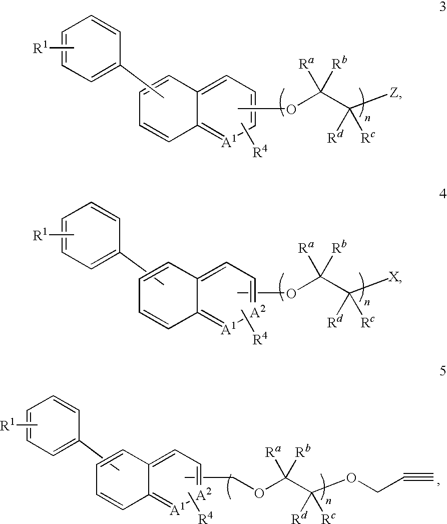

wherein, in any structures containing fragment ii, such as structures 3, 4 and 5, when present, R¹, R⁴, R^{a}, R^{b}, R^{c}, R^{d}, A¹, A², n, Z, y′ and X are as described above. In preferred structures containing fragment ii, the value of y′ is 1 or 0. Also preferred structures, including but not limited to structures 3, 4, and 5, are those where A¹ and A² are each independently C or N; n is an integer from 2 to 6; R¹ is hydroxy, C₁₋₄ alkoxy, —NHCO(C₁₋₄ alkyl) or NR′R″, wherein R′ and R″ are independently hydrogen or (C₁₋₄)alkyl; R⁴ is hydrogen, (C₁₋₄)alkyl, (C₁₋₄)alkoxy, halogen or radiohalogen; and X is hydroxy, halogen, radiohalogen, halo(C₁₋₄)alkyl or radiohalo(C₁₋₄)alkyl; provided that X contains F or ¹⁸F, preferably ¹⁸F, or R⁴ is F, ¹⁸F, ¹²³I, ¹²⁵I, ¹³¹I, ⁷⁶Br, ⁷⁷Br or Br. Also in these embodiments, preferred compounds, including but not limited to structures of 3, 4, and 5, are those where A² is C, n is 3 and/or R^{a}, R^{b}, R^{c} and R^{d} are each hydrogen; alternatively, preferred compounds are where A² is C, n is 1 and R^{a}, R^{b}, and R^{c} are each hydrogen and R^{d} is hydroxy(C₁₋₄)alkyl and Z is X, which is a halo(C₁₋₄)alkyl or, more preferably, radiohalo(C₁₋₄)alkyl.

As shown above, fragment iii is as follows:

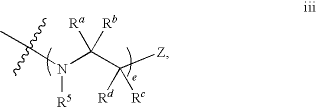

wherein, e is 0 or 1; useful and all preferred values of Z, R^{a}, R^{b}, R^{c}, R^{d} and R⁵ are as described above. Specifically, in a preferred embodiment of Formula I that contains fragment iii, R⁵, R^{a}, R^{b}, R^{c} and R^{d} are each independently as described above; and Z is as described above; provided that if X contains F or ¹⁸F, preferably ¹⁸F, then R⁴ is F, ¹⁸F, ¹²³I, ¹²⁵I, ¹³¹I, ⁷⁶Br, ⁷⁷Br or Br.

Structures of Formula I where A³ and A⁴ are both C and that contain fragment iii include, when e is 1:

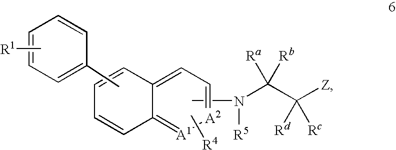

wherein R¹, R⁴, A¹, A², R⁵, R^{a}, R^{b}, R^{c}, R^{d} and Z are as described above; and when e is 0,

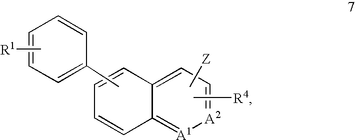

wherein R¹, R⁴, A¹, A² and Z are as described above. In preferred embodiments of structure 6, Z is X, wherein X is hydrogen, halogen, radiohalogen, (C₁₋₄)alkoxy, hydroxy or NR^{x}R^{y}, wherein R^{x} and R^{y} are as described above; or Zc:

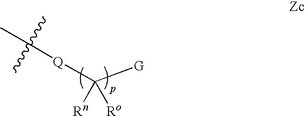

wherein, p is an integer from 1 to 4, Q is O or NR⁵; G is —C═C—(R^{G})X or —C≡C—X, wherein R^{G} is hydrogen or (C₁₋₄)alkyl; R^{a} and R^{o} are independently hydrogen, hydroxyl or (C₁₋₄)alkyl; and X and R⁵ are as described above.

Preferred compounds of Formula I where A³ and A⁴ are both C include:

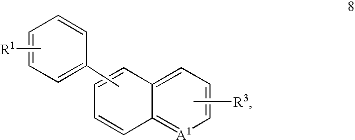

wherein, R¹ is hydroxy or NR′R″, wherein R′ and R″ are as described above, R³ is as described above, and A¹ is C or N;

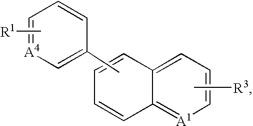

wherein, R¹ is hydroxy or NR′R″, wherein R′ and R″ are as described above, R³ is as described above, A¹ is C or N and A⁴ is N;

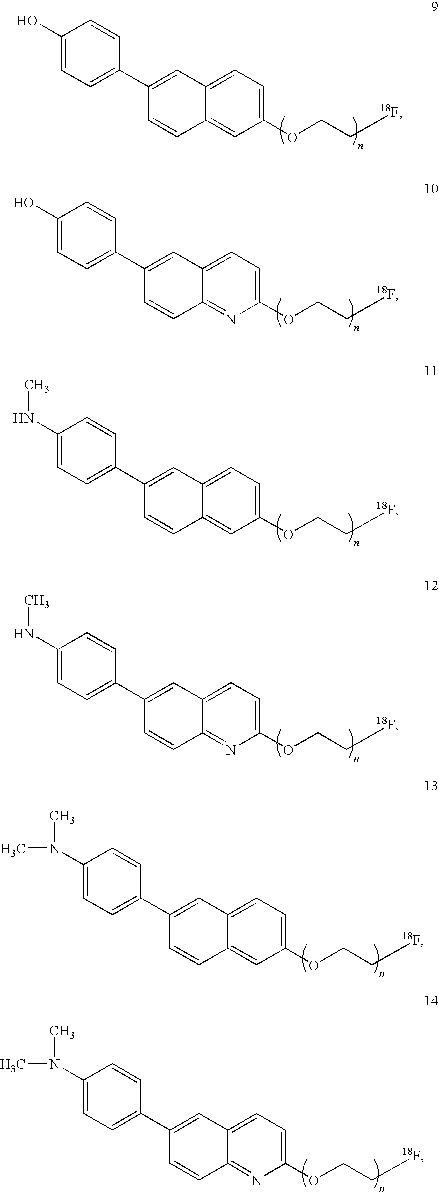

wherein, in compounds 9, 10, 11, 12, 13, 14 and 15, n is an integer from 1 to 10. Preferably, n is from 1 to 6. More preferably, n is from 2 to 6. Most preferably, n is 3;

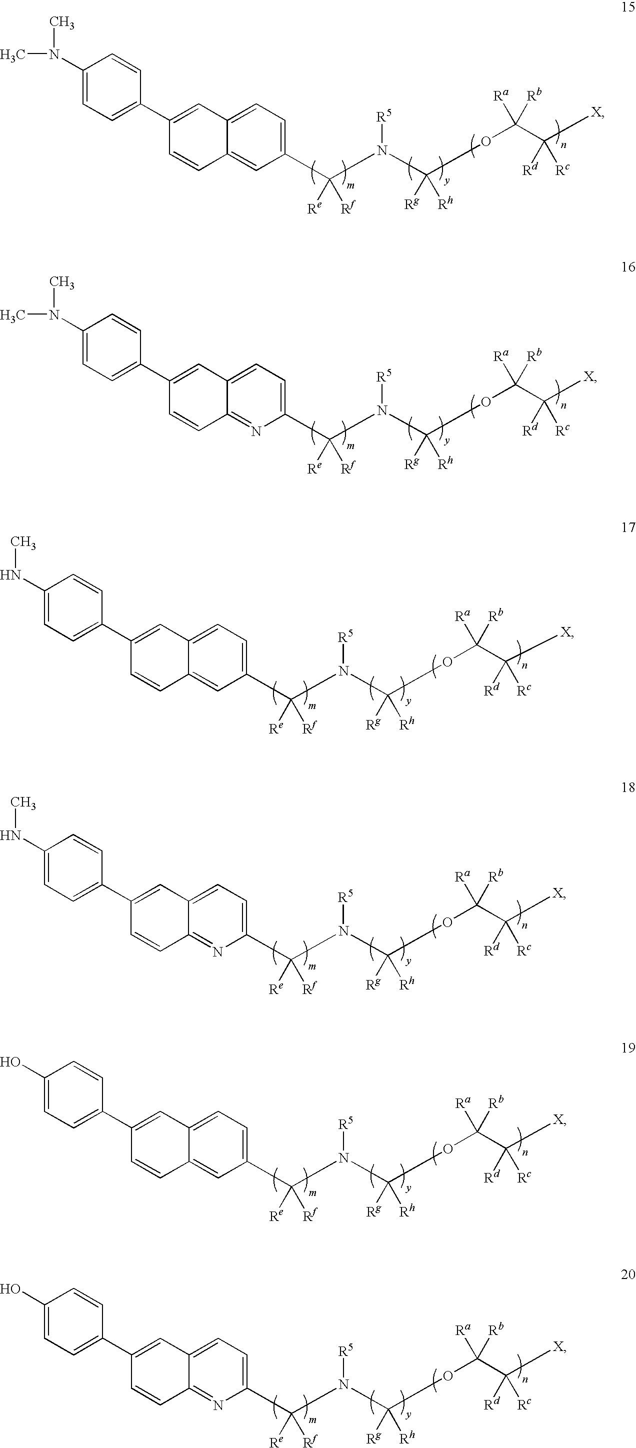

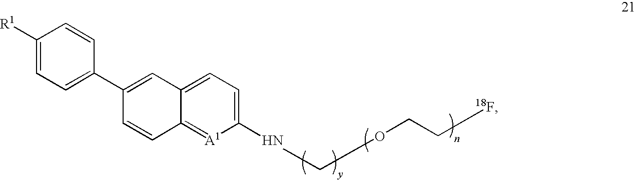

wherein, in any of compounds 15, 16, 17, 18, 19, 20 and 21, when present, m, y, n, R⁵, R^{a}, R^{b}, R^{c}, R^{d} R^{e}, R^{f}, R^{g} and R^{h} are as described above;

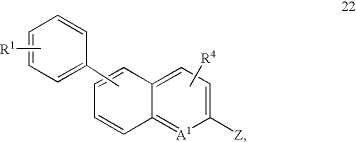

wherein, R¹ is hydroxy or NR′R″, wherein R′ and R″ are independently hydrogen or (C₁₋₄)alkyl, A¹ is C or N, Z is X, wherein X is hydrogen, hydroxy or C₁₋₄ alkoxy and R⁴ is I, ¹²³I, ¹²⁵I, ¹³¹I, Br, ⁷⁶Br or ⁷⁷Br;

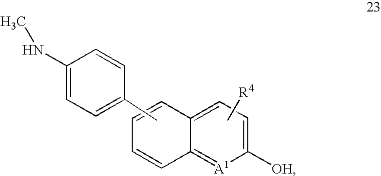

wherein A¹ is C or N, and R⁴ is I, ¹²³I, ¹²⁵I, ¹³¹I, Br, ⁷⁶Br or ⁷⁷Br, more preferably ¹²³I, ⁷⁶Br or ⁷⁷Br;

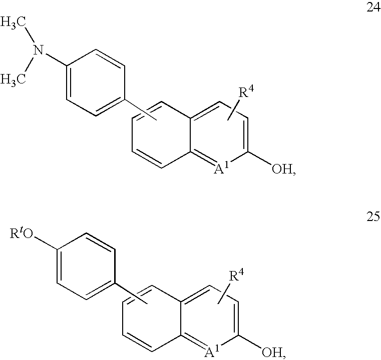

wherein, in compounds 24 and 25, A¹ is C or N, and R⁴ is I, ¹²³I, ¹²⁵I, ¹³¹I, Br, ⁷⁶Br or ⁷⁷Br, more preferably ¹²³I, ⁷⁶Br or ⁷⁷Br. For compound 25, R^{t} is (C₁₋₄)alkyl, preferably methyl, for example, compound 26:

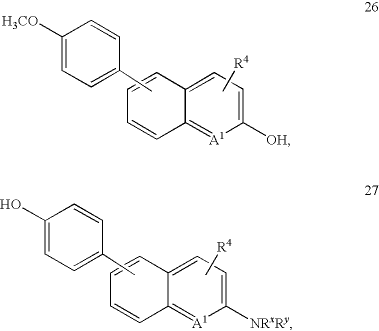

wherein R^{x} and R^{y} are each independently hydrogen or C₁₋₄ alkyl, A¹ is C or N, and R⁴ is F, ¹⁸F, I, ¹²³I, ¹²⁵I, ¹³¹I, Br, ⁷⁶Br or ⁷⁷Br, more preferably ¹²³I, ⁷⁶Br or ⁷⁷Br.

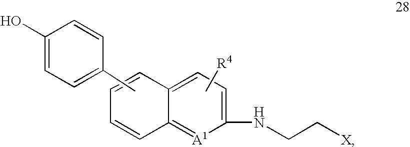

wherein A¹ is C or N, R⁴ is F, ¹⁸F, I, ¹²³I, ¹²⁵I, ¹³¹I, Br, ⁷⁶Br or ⁷⁷Br, more preferably ¹²³I, ⁷⁶Br or ⁷⁷Br, and X is hydroxy, F or ¹⁸F;

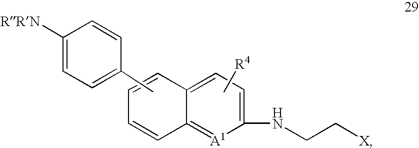

wherein R′ and R″ are each independently hydrogen or (C₁₋₄)alkyl, A¹ is C or N, R⁴ is F, ¹⁸F, I, ¹²³I, ¹²⁵I, ¹³¹I, Br, ⁷⁶Br or ⁷⁷Br, more preferably ¹²³I, ⁷⁶Br or ⁷⁷Br, and X is hydroxy, F or ¹⁸F;

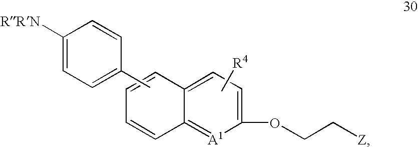

wherein R′ and R″ are each independently hydrogen or (C₁₋₄)alkyl, A¹ is C or N, R⁴ is F, ¹⁸F, ¹²³I, ¹²⁵I, ¹³¹I, Br, ⁷⁶Br or ⁷⁷Br, more preferably ¹²³I, ⁷⁶Br or ⁷⁷Br, and Z is X, wherein X is hydroxy, F or ¹⁸F;

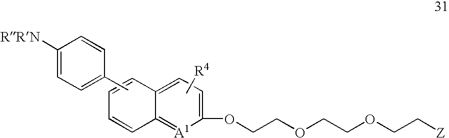

wherein R′ and R″ are each independently hydrogen or (C₁₋₄)alkyl, A¹ is C or N, R⁴ is F, ¹⁸F, I, ¹²³I, ¹²⁵I, ¹³¹I, Br, ⁷⁶Br or ⁷⁷Br, more preferably ¹²³I, ⁷⁶Br or ⁷⁷Br, and Z is as described above. More preferably Z is X, wherein X is hydroxy, F, ¹⁸F or Zc, wherein Zc is:

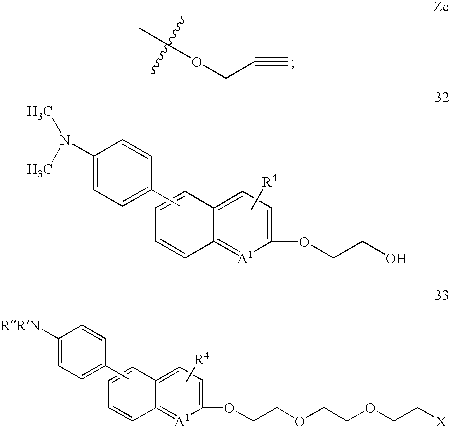

wherein one of R′ and R″ is (C₁₋₄)alkyl, preferably methyl, the other is hydrogen or (C₁₋₄)alkyl, A¹ is C or N, more preferably C, and X is F or ¹⁸F, preferably ¹⁸F;

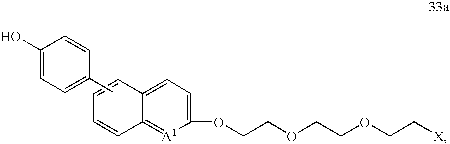

wherein, A¹ is C or N, more preferably C, and X is F or ¹⁸F, preferably ¹⁸F;

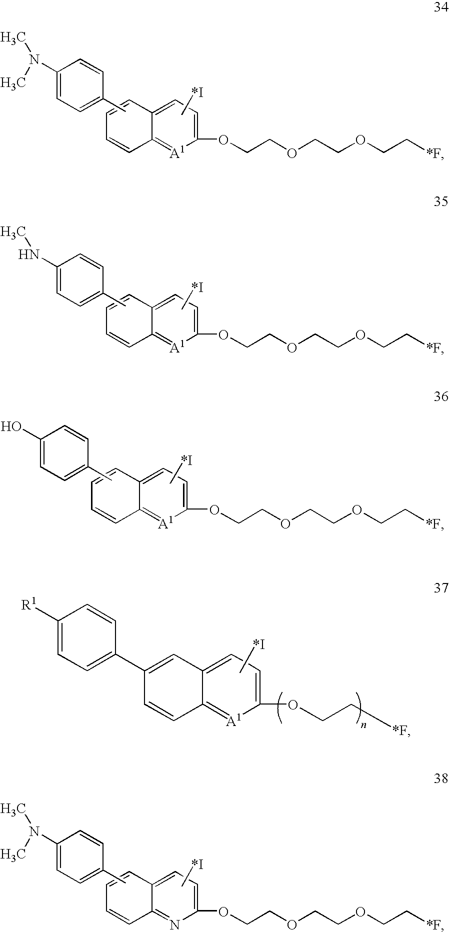

wherein, *I and *F are non-radiolabeled or radiolabeled. Preferably, one of *I and *F is radiolabeled, such as ¹²³I or ¹⁸F. Most preferably, *I is ¹²³I and *F is non-radiolabeled F;

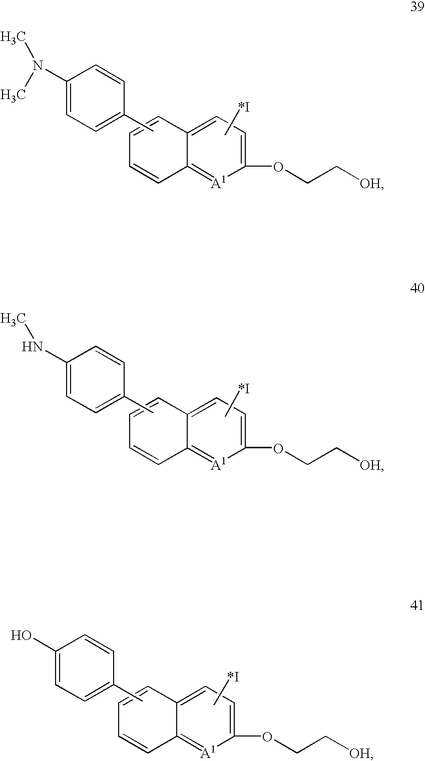

wherein, A¹ is C or N and *I is radiolabeled or non-radiolabeled. Preferably, *I is radiolabeled. Most preferably, *I is ¹²³I;

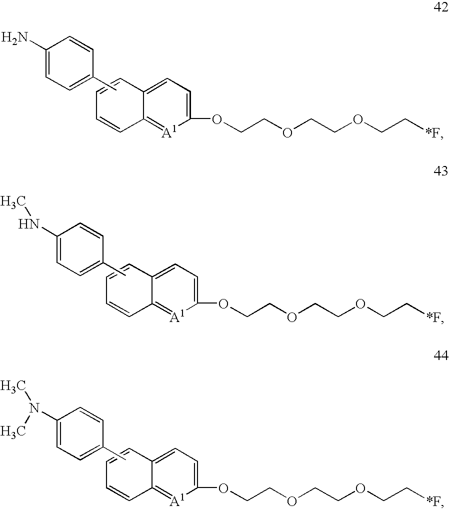

wherein, A¹ is C or N and *F is radiolabeled or non-radiolabeled fluorine. Preferably, *F is ¹⁸F; and

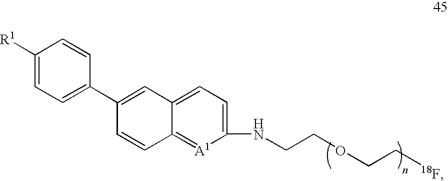

wherein, R¹ is —N(Me)₂, —NHMe or hydroxy, A¹ is C or N and n is 1, 2 or 3.

Other compounds of the present invention include hydroxy branched derivatives such as,

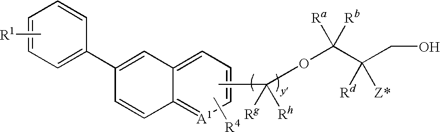

wherein, R¹ includes all useful values of R¹ described herein, A¹ is C or N, y′ is an integer from 0 to 5, R⁴, R^{a}, R^{b}, R^{c}, R^{d}, R^{g} and R^{h} include all useful values for these R groups as described herein, and Z* is Z or Z′, which is described fully below under Formula II compounds. Especially preferred compounds include those where Z* is a radiohalo(C₁₋₄)alkyl, including but not limited to ¹⁸Fluoromethyl, exemplified as follows:

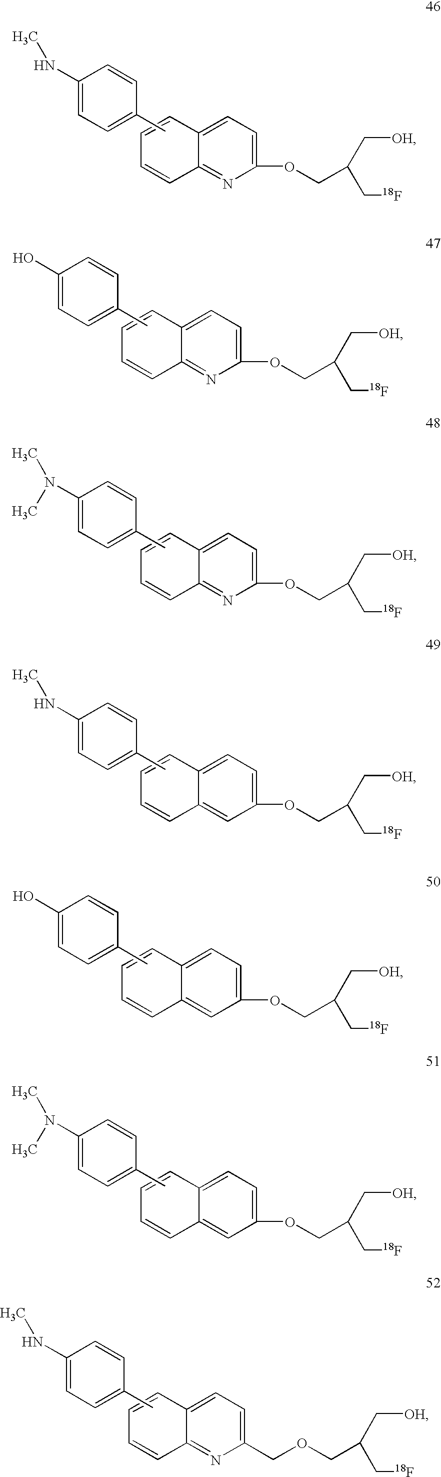

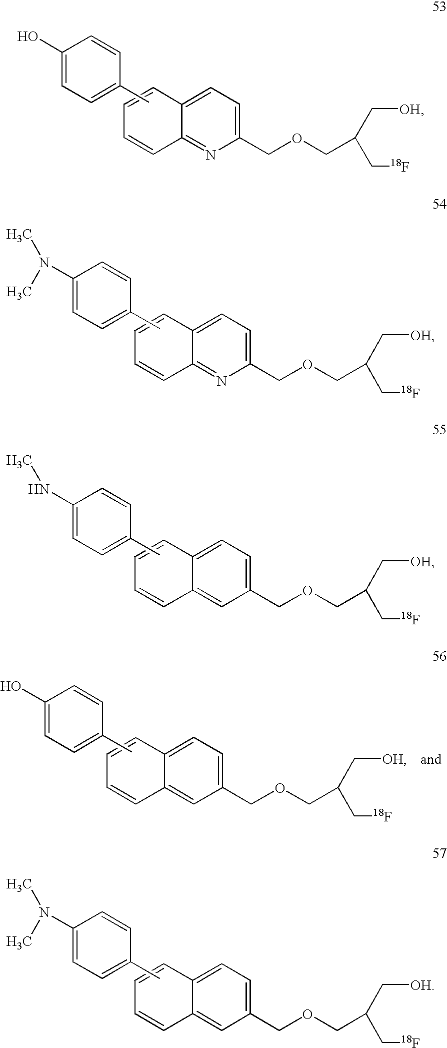

In all embodiments under Formula I, it is more preferable that the phenyl and the naphthalene ring systems are in the following configuration relative to one another:

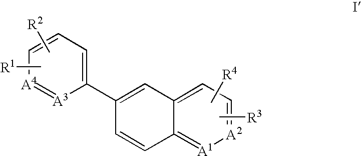

wherein, R¹, R², R³, R⁴, A¹ and A² are as described in all above embodiments.

Also preferred in all of the above embodiments and structures of Formula I and I′ are compounds where A⁴ is N.

The present invention is also directed to compounds having the following structure, Formula II:

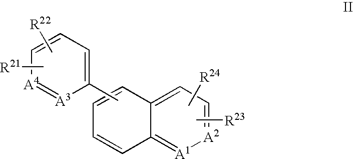

or a pharmaceutically acceptable salt or prodrug thereof, wherein:

A¹, A², A³ and A⁴ are independently C or N;

R²¹ and R²⁴ are each independently:
NR′R″, wherein R′ and R″ are independently hydrogen, (C₁₋₄)alkyl, hydroxy(C₁₋₄alkyl or halo(C₁₋₄)alkyl;
hydroxy;
C₁₋₄ alkoxy;
hydroxy(C₁₋₄alkyl;
halogen;
cyano;
hydrogen;
nitro;
(C₁-C₄)alkyl;
halo(C₁-C₄)alkyl;
formyl;
—O—CO(C₁₋₄ alkyl);
—COO(C₁₋₄ alkyl);
—NHCO(C₁₋₄ alkyl);or
radiohalogen;

R²² and R²³ are hydrogen or fragment i, ii, iii or iv, wherein:

fragment i is:

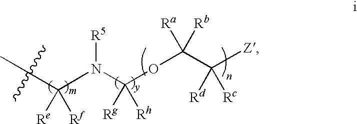

wherein, n is an integer from 1 to 10; m is an integer from 0 to 5; y is an integer from 1 to 5; R⁵ is hydrogen, (C₁₋₄)alkyl, or hydroxy(C₁₋₄alkyl; R^{a}, R^{b}, R^{c}, R^{d}, R^{e}, R^{f}, R^{g} and R^{h} are each independently hydrogen, halogen, hydroxy, (C₁₋₄)alkoxy, (C₁₋₄)alkyl or hydroxy(C₁₋₄alkyl; and Z′ is:
   a) -Ch, wherein -Ch is described fully below;
   b) one of the following groups, each of which contains a -Ch directly bound to the aromatic ring: benzoyloxy, phenyl(C₁₋₄)alkyl, aryloxy and C₆₋₁₀ aryl; or
   c) Z′c, having the following structure:

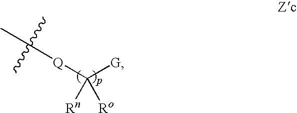

wherein, p is an integer from 1 to 4, Q is O or NR⁵; G is —C═C—(R^{G})Ch or —C≡C-Ch, wherein R^{G} is hydrogen or (C₁₋₄)alkyl; R^{a} and R^{o} are independently hydrogen, hydroxy or (C₁₋₄)alkyl, R⁵ is as described herein and Ch is as described below;

fragment ii is:

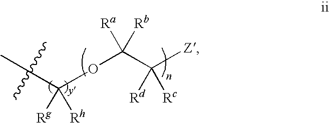

wherein, n, R^{a}, R^{b}, R^{c}, R^{d}, R^{g} and R^{h} and Z′ are as described above. The values of y′ are those shown above for fragment ii under Formula I;

fragment iii is:

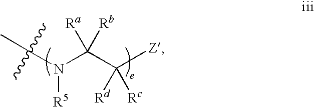

wherein, e is 0 or 1, and Z′, R^{a}, R^{b}, R^{c}, R^{d} and R⁵ are as described above;

and fragment iv is:

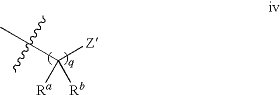

wherein Z′, R^{a} and R^{b} are as described above, and q is an integer from 1 to 10;
   or R²³ and R²⁴ taken together form -Ch, provided that one of R²² and R²³ is other than hydrogen.

The moiety “-Ch” is a chelating ligand capable of complexing with a metal to form a metal chelate. Many ligands are known in the art and are suitable for use as a labeling moiety for compounds of Formula II. Those of skill in the art will understand that such ligands provide a convenient way to label compounds and the invention is not limited to particular ligands, many of which are interchangeable. Preferably, this ligand is a tri- or tetradentate ligand, such as N₃, N₂S, NS₂, N₄ and those of the N₂S₂ type, such as:

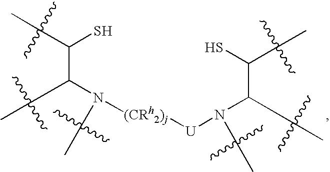

wherein, [symbol] indicates a possible point(s) of attachment of the ligand to the backbone of the amyloid binding structure, j is 0, 1 or 2; and U is two adjacent carbons on the aromatic ring of the backbone or —C(R³⁵R³⁶)C(R³⁷R³⁸)—; wherein R^{h}, in each instance, and R³⁵, R³⁶, R³⁷ and R³⁸ are independently hydrogen, hydroxy, amino, methylamino, dimethylamino, (C₁₋₄)alkoxy, (C₁₋₄)alkyl, and hydroxy(C₁₋₄alkyl. The preferred values for these particular R groups are hydrogen and C₁₋₄ alkyl.

The above ligand can be substituted at other positions if available:

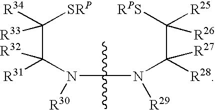

Other potentially available positions are represented by R²⁵, R²⁶, R²⁷, R²⁸, R²⁹, R³⁰, R³¹, R³², R³³ and R³⁴. One or two of these R group(s) will not be available when the ligand is attached to the backbone at that particular position(s). When available, these R groups are independently selected from the group consisting of hydrogen, hydroxy, amino, methylamino, dimethylamino, (C₁₋₄)alkoxy, (C₁₋₄)alkyl, and hydroxy(C₁₋₄)alkyl. Preferably, the R groups are hydrogen or (C₁₋₄)alkyl.

Both R^{P} groups can be hydrogen, or can be any of the variety of protecting groups available for sulfur, including methoxymethyl, methoxyethoxymethyl, p-methoxybenzyl or benzyl. Sulfur protecting groups are described in detail in, for example, Greene, T. W. and Wuts, P. G. M., Protective Groups in Organic Synthesis, 2nd Edition, John Wiley and Sons, Inc., New York (1991). Protecting group R^{P} can be removed by appropriate methods well known in the art of organic synthesis, such as trifluoroacetic acid, mercuric chloride or sodium in liquid ammonia. In the case of Lewis acid labile groups, including acetamidomethyl and benzamidomethyl, R^{P} can be left intact. Labeling of the ligand with technetium in this case will cleave the protecting group, rendering the protected diaminedithiol equivalent to the unprotected form.

The metal ligand moiety is capable of complexing with a radiometal, such as ^{99m}Tc, to form a metal chelate as exemplified by the following structure:

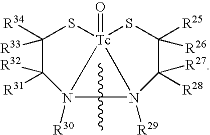

Additionally, other radiometals can be complexed with the ligand, such as rhenium.

Preferred values of R²¹ include hydroxy, C₁₋₄ alkoxy, —NHCO(C₁₋₄ alkyl) and NR′R″, wherein R′ and R″ are as described above. More preferably, R²¹ is hydroxy or NR′R″, wherein R′ and R″ are independently hydrogen or C₁₋₄ alkyl. The more preferred value of (C₁₋₄)alkyl in these embodiments is methyl.

Preferably, R²⁴ is hydrogen, halogen or (C₁₋₄)alkyl.

Useful values of A¹, A², A³ and A⁴ are independently C, CH, and N. Preferably, one of A¹ and A² is C, and the other is C or N. When one of A¹ and A² is N, it is more preferred that A², which is in the meta position relative to the alkene bridge, is N. Preferably, both A³ and A⁴ are C. In another preferred embodiment, one of A³ and A⁴ is N. When one of A³ and A⁴ is N, it is more preferred that A³, which is in the meta position relative to the alkene bridge, is N. In especially preferred embodiments, A¹ is C, A² is C or N, A³ is C and A⁴ is C or N.

Useful values of R²² and R²³ include fragments i, ii, iii and iv. Each of these fragments contains a Z′ group. Each Z′ group, as shown above, contains a -Ch moiety. The -Ch moiety as described fully herein is a chelating moiety capable of complexing with a metal to form a chelate. Fragments i, ii, iii and iv are discussed more fully below.

As shown above, fragment i is as follows:

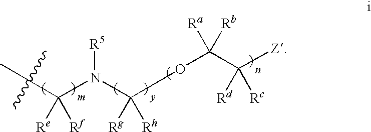

In all embodiments, useful values of n are integers from 1 to 10. Preferably, n is an integer from 1 to 6. More preferably, n is an integer from 2 to 6, and most preferably, n is 3. In all embodiments, useful values of m are integers from 0 to 5. Preferably, m is an integer from 0 to 3. More preferably, m is 0 or 1, and most preferably m is 0. In all embodiments, useful values of y are integers from 0 to 5. Preferably, y is an integer from 0 to 3. More preferably, y is an integer from 0 to 2, and most preferably, y is 2. In all embodiments, R⁵ is hydrogen, (C₁₋₄)alkyl or hydroxy(C₁₋₄alkyl. More preferably, R⁵ is hydrogen or C₁₋₄ alkyl. Most preferably, R⁵ is hydrogen. In all embodiments, R^{a}, R^{b}, R^{c}, R^{d}, R^{e}, R^{f}, R^{g} and R^{h} are independent of one another and are hydrogen, halogen, hydroxy, (C₁₋₄)alkoxy, (C₁₋₄)alkyl or hydroxy(C₁₋₄alkyl, preferably, hydrogen, hydroxy or (C₁₋₄)alkyl. More preferably R^{a}, R^{b}, R^{c}, R^{d}, R^{e}, R^{f}, R^{g} and R^{h} are independent of one another and are hydrogen or (C₁₋₄)alkyl, most preferably, hydrogen. In all embodiments, Z′ is:

a) -Ch, wherein -Ch is as described herein;

b) one of the following groups, each of which contains a -Ch directly bound to the aromatic ring: benzoyloxy, phenyl(C₁₋₄)alkyl, aryloxy and C₆₋₁₀ aryl; or

c) Z′c, having the following structure:

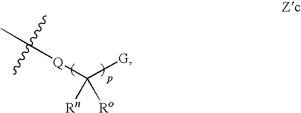

wherein, p is an integer from 1 to 4, preferably 2, Q is O or NR⁵ and G is —C═C—(R^{G})Ch or —C≡C-Ch, wherein R^{G} is hydrogen or C₁₋₄ alkyl; Rⁿ and R^{o} are independently hydrogen, hydroxy or C₁₋₄ alkyl, and Ch is as described herein.

As shown above, fragment ii is:

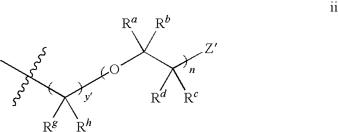

wherein, n, R^{a}, R^{b}, R^{c}, R^{d}, R^{g}, R^{h}, y′ and Z′ are as described above. In compounds of Formula II, R^{a}, R^{b}, R^{c} and R^{d} are preferably (C₁₋₄)alkyl or hydrogen, more preferrably hydrogen. y′ is preferably an integer from 0 to 3. Most preferably, the y′ is 0 or 1. n is an integer from 1 to 10. Preferably, n is from 2 to 6. Most preferably, n is 3. Preferably, Z′ is -Ch. In these embodiments, -Ch is more preferably a N₂S₂ type ligand.

As shown above, fragment iii is:

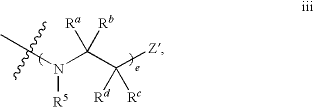

wherein, e is 0 or 1, and Z′, R^{a}, R^{b}, R^{c}, R^{d} and R⁵ are as described above. In compounds of Formula II, R^{a}, R^{b}, R^{c} and R^{d} are preferably (C₁₋₄)alkyl or hydrogen, more preferably hydrogen. Preferably, Z′ is -Ch. In these embodiments, -Ch is more preferably a N₂S₂ type ligand.

As shown above, fragment iv is:

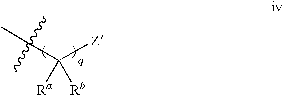

wherein Z′, R^{a} and R^{b} are as described above, and q is an integer from 1 to 10;

or R²³ and R²⁴ taken together form -Ch. In compounds of Formula II, R^{a} and R^{b} are preferably C₁₋₄ alkyl or hydrogen. The more preferred value is hydrogen. Preferred values of q are integers from 1 to 6. Preferably, q is an integer from 1 to 4. Preferably, Z′ is -Ch. In this embodiment, -Ch is more preferably a N₂S₂ type ligand.

Examples of compounds of Formula II where A³ and A⁴ are both C include:

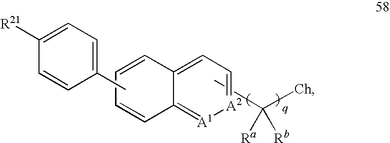

wherein, R²¹ is hydroxyl, mono- or di(C₁₋₄)amino; A¹ and A² are C or N; R^{a} and R^{b} in each instance are independently hydrogen or (C₁₋₄)alkyl; -Ch is as described herein; and q is an integer from 1 to 6;

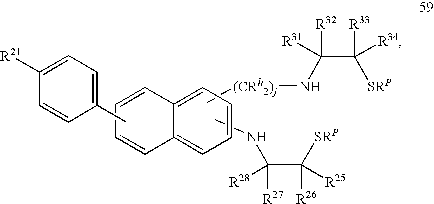

R²¹ is hydroxyl, mono- or di(C₁₋₄)amino; A¹ and A² are C or N; R^{a} and R^{b} in each instance are independently hydrogen or C₁₋₄alkyl; R²⁵ through R³⁴ are in each instance independently hydrogen or C₁₋₄alkyl; and q is an integer from 1 to 6;

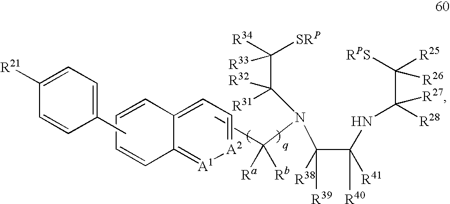

wherein R²¹ is hydroxyl, mono- and di(C₁₋₄)amino; A¹ and A² are C or N; R^{h} in each instance are independently hydrogen or (C₁₋₄)alkyl; j is 1 or 2; and R²⁵ through R³⁴ are in each instance hydrogen or (C₁₋₄)alkyl. The present invention includes the complexes when compounds such as 59 and 60 are complexed with a radiometal such as ^{99m}Tc. A non-limiting example has the following radiometal complex:

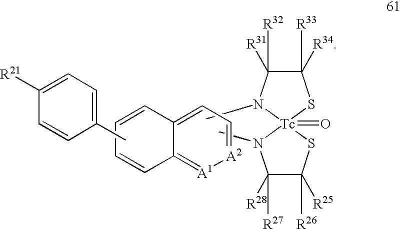

In all embodiments of Formula II, it is more preferable that the phenyl and the naphthalene ring systems are in the following configuration relative to one another:

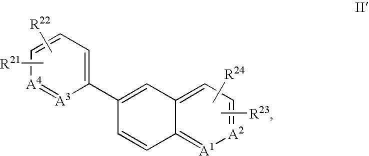

wherein, R²¹, R²², R²³, R²⁴, A¹ and A² are as described in all above embodiments.

Also preferred in all of the above embodiments and structures of Formula II and II′ are compounds where A⁴ is N.

It is also to be understood that the present invention is considered to include stereoisomers. Further included are: optical isomers, e.g. mixtures of enantiomers as well as individual enantiomers and diastereomers, which may arise as a consequence of structural asymmetry in selected compounds of Formula I, I′, II or II′.

When any variable occurs more than one time in any constituent or in Formula I, I′, II or II′ its definition on each occurrence is independent of its definition at every other occurrence. Also combinations of substituents and/or variables are permissible only if such combinations result in stable compounds.

The compounds of Formula I, I′, II or II′ may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The present invention is further directed to methods of preparing compounds of the above Formula I, I′, II or II′. Synthetic routes for preparing compounds of the present invention are described in the following Schemes. The following references were consulted regarding certain of the synthetic sequences: Leigh C. Anderson and Donald G. Thomas: Quinoidation of Triaryl Compounds—Hydroxynaphthyldiphenylcarbinols J. Am. Chem. Soc.; 65; 1943; 239, 241; Cox, D. P.; etc. J. Org. Chem. 49; 1984; 3216-3219.

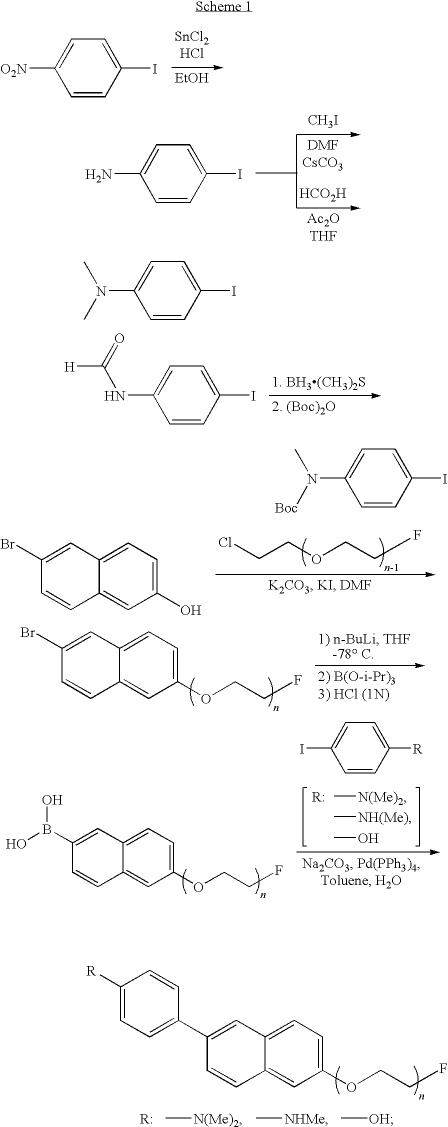

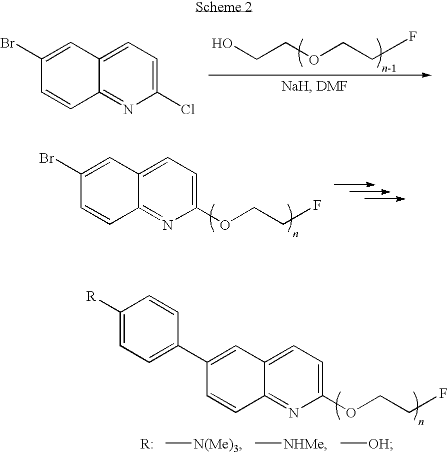

Schemes 3, 4, 6, 7 and 8 depict synthetic routes to compounds exemplified by compounds 15-21 and 45.

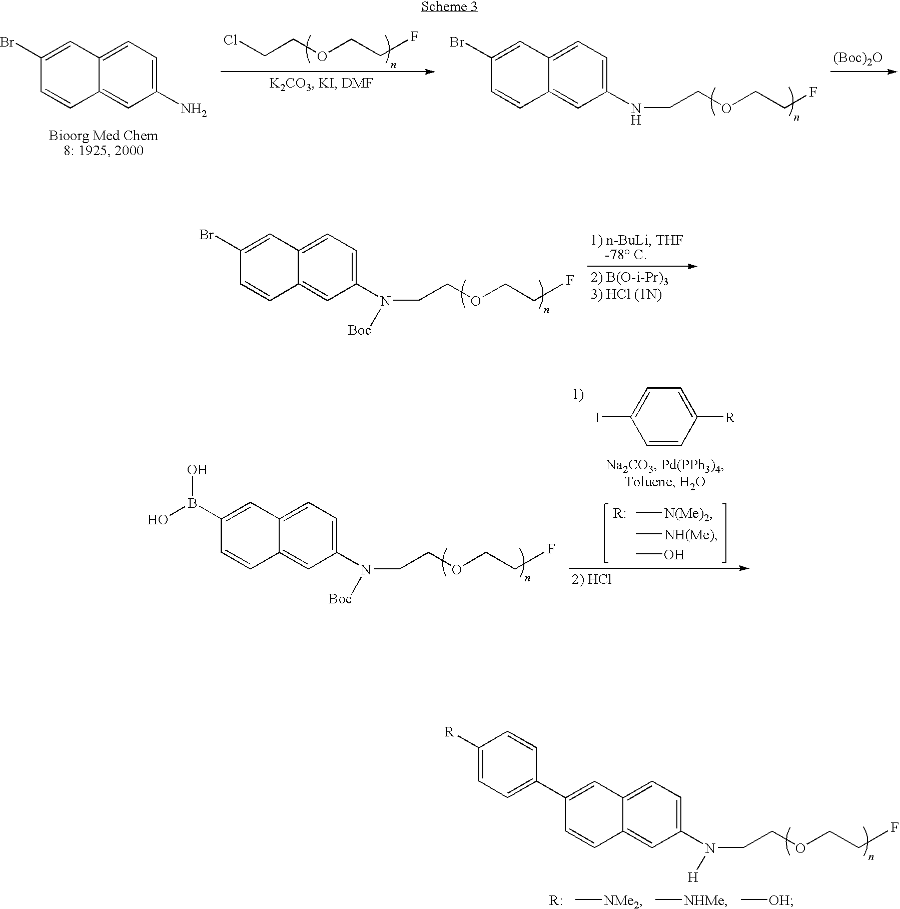

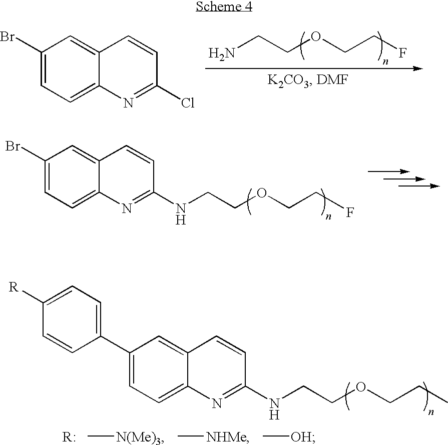

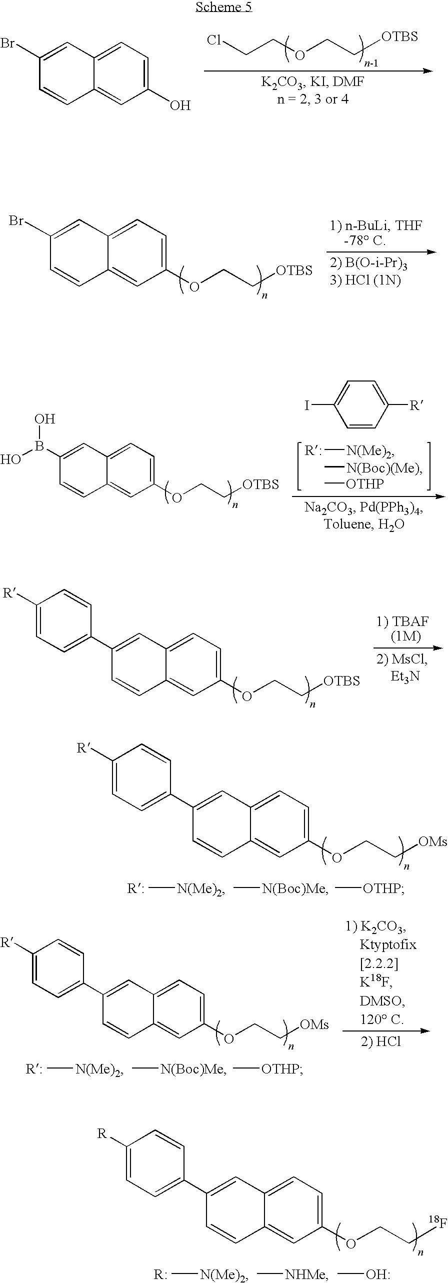

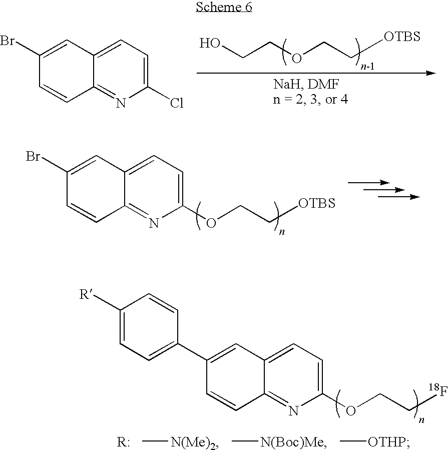

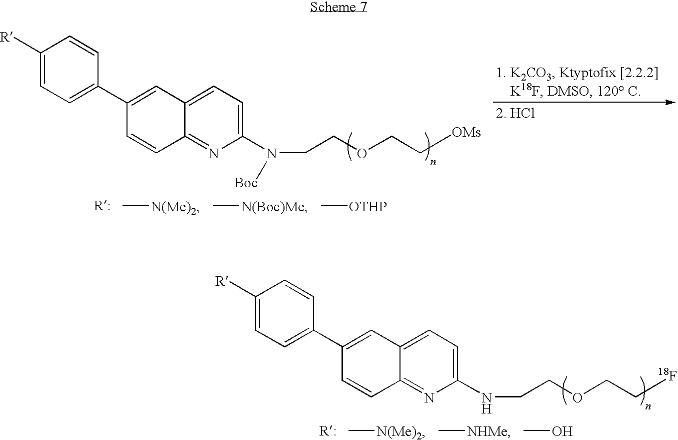

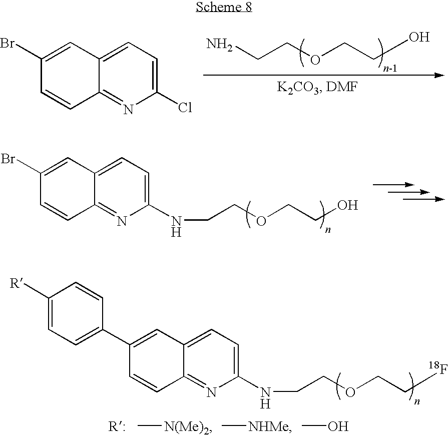

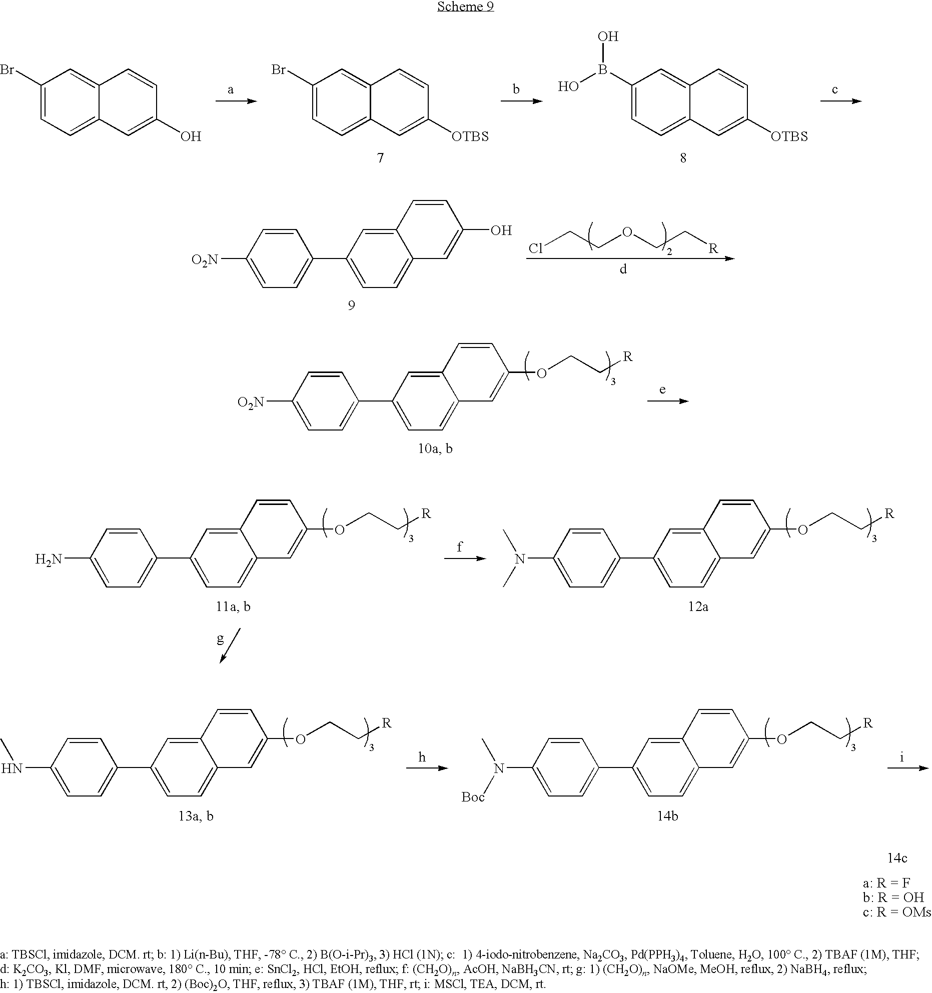

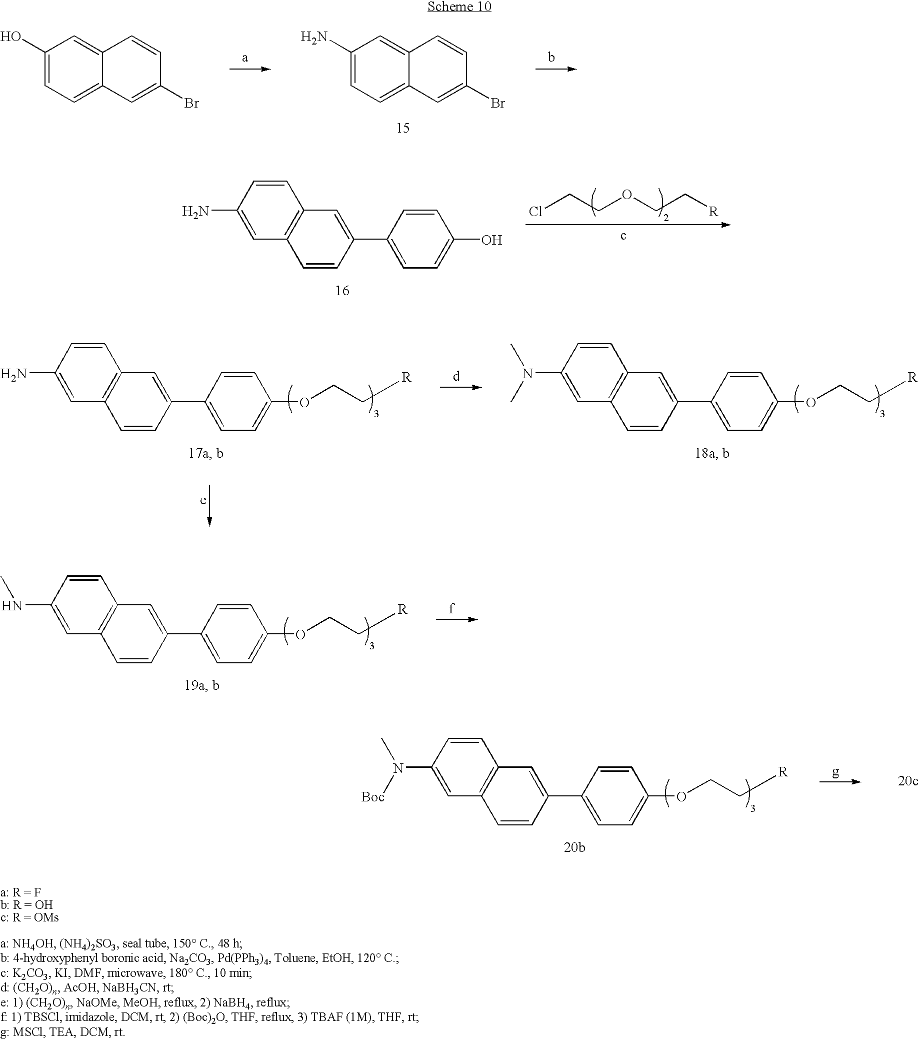

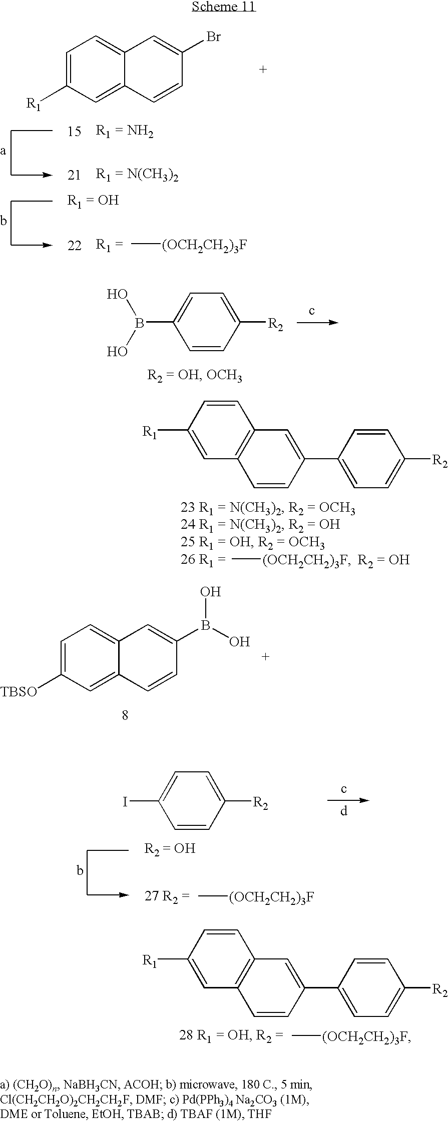

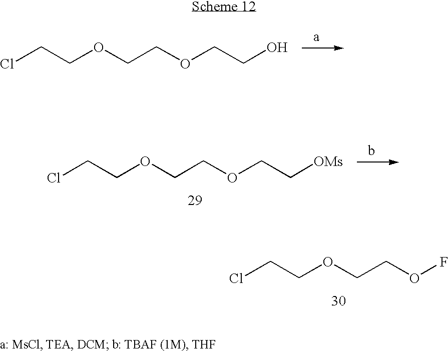

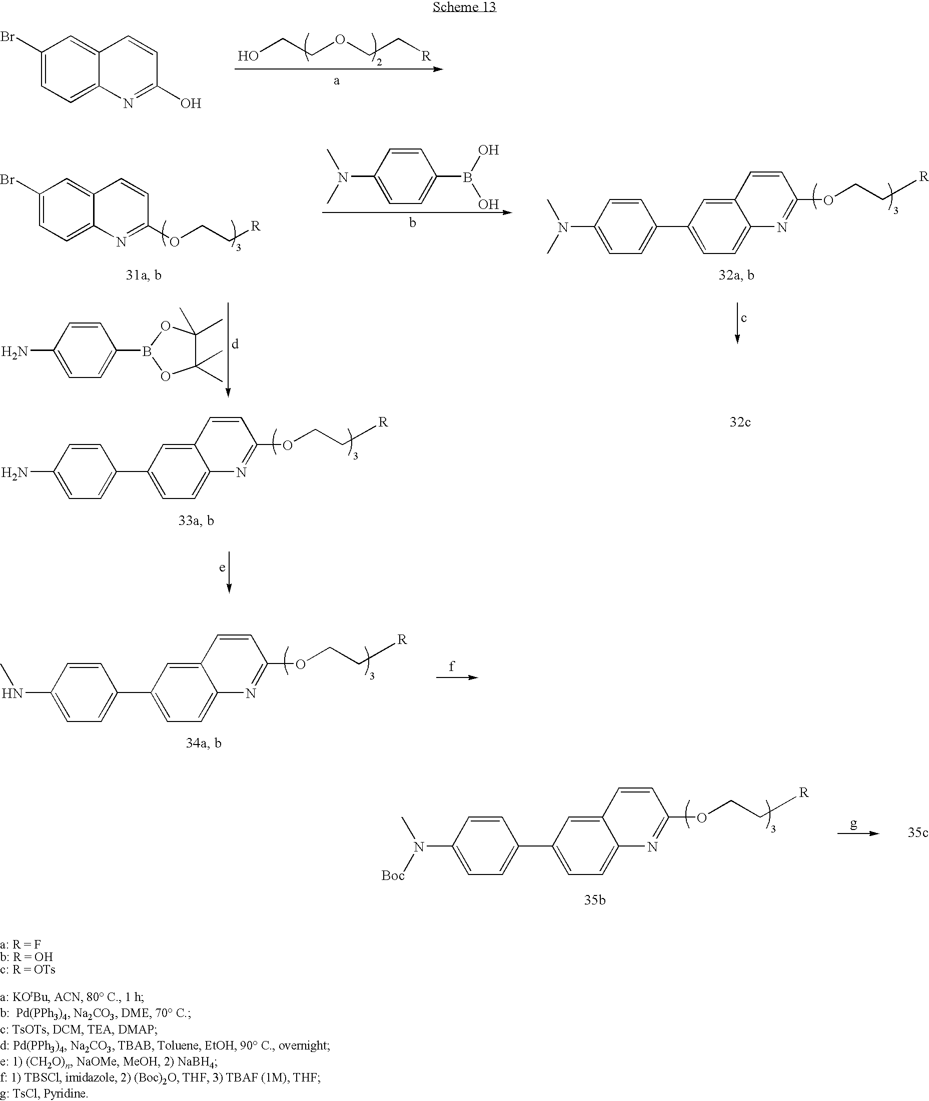

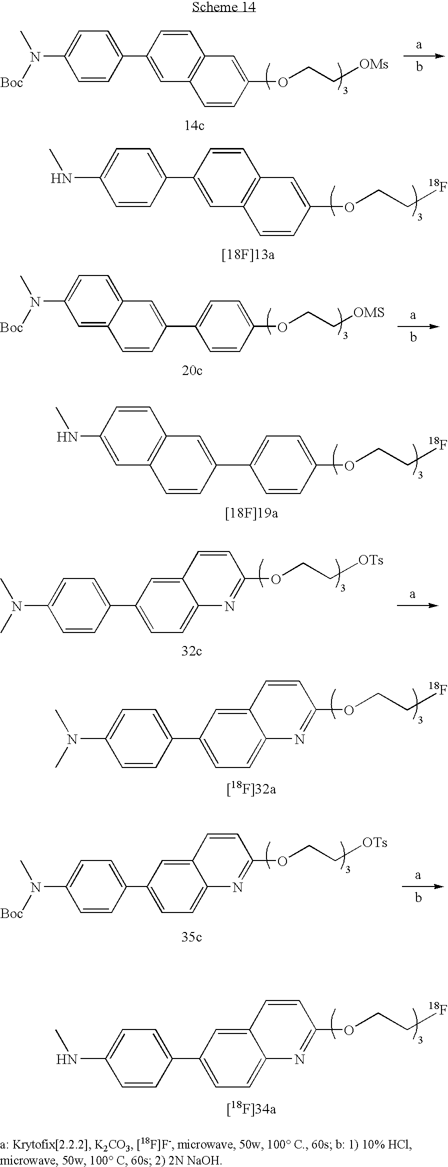

The hydroxy group of 6-bromonaphthalen-2-ol was converted to boronic acid 8 (Yield: 53%) by first protecting the phenol group with TBS and then reacting with n-butyl lithium and triisopropylborate at −78° C. Suzuki coupling of compound 8 with para-iodonitrobenzene in toluene followed by deprotection of TBS group with TBAF(1M in THF) gave compound 9 (Yield: 81.8%). The alkylation of the hydroxy group of compound 9 with either 1-chloro-2-(2-(2-fluoroethoxy)ethoxy)ethane (30)^{a} or 2-(2-(2-chloroethoxy)ethoxy)ethanol was conducted using Biotage Initiated microwave system (180° C., 10 min, high absorption level) to obtain compound 10a (Yield: 89%) or 10b (Yield: 81%) respectively. The nitro group of 10a,b was reduced to amine with stannous chloride in ethanol to give 11a,b (Yield: 76%, 81%). The dimethylated derivative 12a was obtained by reacting 11a with paraformaldehyde at the presence of sodium cyanoborohydride in acetic acid at 96% yield. Monomethylation of 11a,b was achieved using paraformaldehyde, sodium methoxide and sodium borohydride to afford 13a,b (Yield: 72%, 88%). The hydroxy group of 13b was protected with TBS before protecting the monomethyl amino group with Boc. TBS was then removed by TBAF (1M in THF) to obtain compound 14b (Yield: 57% for three steps based on 13b), which was converted into mesylate using methansulfonyl chloride and triethyl amine in dichloromethane to give compound 14c (Yield: 90%), which was used as the labeling precursor for preparing [¹⁸F]12a.

Bromonaphthalen-2-amine (15) was obtained by converting the hydroxy group of 6-bromonaphthalen-2-ol into amine. (Yield: 94%). Suzuki coupling of 15 with 4-hydroxyphenylboronic acid gave compound 16 (Yield: 71%), which was barely soluble in methanol and was purified by washing off organic impurities with methanol. The hydroxy group of 16 was then alkylated with either 1-chloro-2-(2-(2-fluoroethoxy)ethoxy)ethane (30)^{a} or 2-(2-(2-chloroethoxy)ethoxy)ethanol under the same microwave condition for preparing of 10a,b to gave 17a,b (Yield: 72%, 73%, respectively). Same dimethylation and monomethylation approaches for making the dimethylamino and monomethylamino phenylnaphthalene derivatives were applied on 17a,b to give the corresponding dimethylamino-naphthalene-phen derivatives 18a,b (Yield: 48%, 50%) and monomethyl-naphthalene-phen derivatives 19a,b (Yield: 84%, 94%). To make the mesylate 20c, compound 19b was first converted into 20b (Yield: 66.4%) through a similar procedure used for preparing 14c: TBS protection of the hydroxyl group, Boc protection of the methyl amino group and then remove the TBS protection group with TBAF(1M)/THF solution. The free OH group of 20b was then reacted with methansulfonyl chloride to give 20c (Yield: 95%).

Other Phen-Naphthalene or Naphthalene-Phen derivatives (23, 24, 25, 26, 28) were synthesized using Suzuki coupling in DME or a mix-solvent of toluene and ethanol with corresponding starting materials, which were either commercial available or prepared as showing in Scheme 3 (Yield: 34%-71%).

The hydroxyl group of 6-bromo-2-hydroxy-quinolin was alkylated by 2-(2-(2-fluoroethoxy)ethoxy)ethanol or by triethylene glycol with potassium tert-butoxide at 80° C. in acetonitrile to afford 31a (74%) or 31b (91%) respectively. Suzuki coupling of 31a or 31b was performed in DME at 70° C. with 4-dimethylamino-boronic acid, palladium tetrakistriphenylphosphine and sodium carbonate to give compound 32a (30%) or 32b (94%) respectively. Tosylation of 32b with tosyl chloride in pyridine was not successful, but using tosyl anhydride in dichloromethane at the presence of triethyl amine and catalytic amount of DMAP, 32b was successfully converted into 32c in a very good yield (83.4%). Suzuki coupling of 31a or 31b with 4-aminophenylboronic acid pinacolate, was conducted in toluene at the presence of palladium tetrakistriphenylphosphine, tetrabutyl ammonium bromide, and sodium carbonate to yield 33a (75%) or 33b (89%) respectively. Monomethylation of 33a, b using paraformaldehyde, sodium methoxide and sodiumborohydride obtained 34a, b (Yield: 72%, 91.5%). 34b was then converted into 35b through the following steps: TBS protection of the hydroxyl group, Boc protection of the methyl amino group and then remove the TBS protection group. The free hydroxy group of 35b was converted into tosylate by reacting with tosyl chloride in pyridine at room temperature to yield 35c (43%).

To make F-18 labeled Phen-Nap, Nap-Phen and Phen-Quinoline derivatives [¹⁸F]13a, [¹⁸F]19a, [¹⁸F]32a and [¹⁸F]34a, compound 14c, 20c, 32a and 35c were used as the corresponding precursors (Scheme 6). Each of the mesylate or tosylate 14c, 20c, 32c and 35c was mixed with [¹⁸F]/F⁻, Kryptofix[2.2.2], potassium carbonate in DMSO and applied for microwave irradiation at 50 Watts, maximum 100° C., for 60 sec. For making [¹⁸F]32a, the crude product was purified by semi-preparative HPLC immediately after the labeling reaction. For making [¹⁸F] 13a, [¹⁸F] 19a, [¹⁸F]34a, the crude product was added 10% HCl and irradiated with microwave at 50 Watts, maximum 100° C., for 60 sec to remove Boc protecting group and then neutralized with sodium hydroxide followed by semi-preparative HPLC purification. The preparation of [¹⁸F]13a, [¹⁸F]19a, [¹⁸F]32a and [¹⁸F]34a took about 50-70 min, the radiochemical yield was about 30% (decay corrected), radiochemical purity was >99% and the specific activity (SA) was estimated to be 500-2000 Ci/mmol at the end of synthesis.

The present invention is also directed at a method of imaging amyloid deposits. When the compounds of this invention are to be used as imaging agents, they must be labeled with suitable radioactive isotopes, such as radioactive halogens, radioactive metals and other detectable radioactive atoms such as ¹¹C.

Regarding radiohalogens, ¹²⁵I-isotopes are useful for laboratory testing, but they will generally not be useful for actual diagnostic purposes because of the relatively long half-life (60 days) and low gamma-emission (30-65 Key) of ¹²⁵I. The isotope ¹²³I has a half life of thirteen hours and gamma energy of 159 KeV, and it is therefore expected that labeling of ligands to be used for diagnostic purposes would be with this isotope or ¹⁸F (half life of 2 hours). Other isotopes which may be used include ¹³¹I. Suitable bromine isotopes include ⁷⁷Br and ⁷⁶Br.

The compounds of the present invention can also contain a radioactive isotope of carbon as the radiolabel. This refers to a compound that comprises one or more radioactive carbon atoms, preferably ¹¹C, with a specific activity above that of the background level for that atom. It is well known, in this respect, that naturally occurring elements are present in the form of varying isotopes, some of which are radioactive isotopes. The radioactivity of the naturally occurring elements is a result of the natural distribution or abundance of these isotopes, and is commonly referred to as a background level. The carbon labeled compounds of the present invention have a specific activity that is higher than the natural abundance, and therefore above the background level. The composition claimed herein comprising a carbon-labeled compound(s) of the present invention will have an amount of the compound such that the composition can be used for tracing, imaging, radiotherapy, and the like.

Examples of suitable radioactive metals are disclosed herein. A particularly useful radioactive metal is Tc-99m. Tc-99m complexes can be prepared as follows. A small amount of non-radiolabeled compound (1-2 mg) is dissolved in 100 μL EtOH and mixed with 200 μL HCl (1 N) and 1 mL Sn-glucoheptonate solution (containing 8-32 μg SnCl₂ and 80-320 μg Na-glucoheptonate, pH 6.67) and 50 μL EDTA solution (0.1 N). [^{99m}Tc]Pertechnetate (100-200 μL; ranging from 2-20 mCi) saline solution are then added. The reaction is heated for 30 min at 100° C., then cooled to room temperature. The reaction mixture is analyzed on TLC (EtOH:conc. NH₃ 9:1) for product formation and purity check. The mixture can be neutralized with phosphate buffer to pH 5.0.

The present invention further relates to a method of preparing a technetium-99m complex according to the present invention by reacting technetium-99m in the form of a pertechnetate in the presence of a reducing agent and optionally a suitable chelator with an appropriate Ch-containing compound.

The reducing agent serves to reduce the Tc-99m pertechnetate which is eluted from a molybdenum-technetium generator in a physiological saline solution. Suitable reducing agents are, for example, dithionite, formamidine sulphinic acid, diaminoethane disulphinate or suitable metallic reducing agents such as Sn(II), Fe(II), Cu(I), Ti(III) or Sb(III). Sn(II) has proven to be particularly suitable.

For the above-mentioned complex-forming reaction, technetium-99m is reacted with an appropriate compound of the invention as a salt or in the form of technetium bound to comparatively weak chelators. In the latter case the desired technetium-99m complex is formed by ligand exchange. Examples of suitable chelators for the radionuclide are dicarboxylic acids, such as oxalic acid, malonic acid, succinic acid, maleic acid, orthophtalic acid, malic acid, lactic acid, tartaric acid, citric acid, ascorbic acid, salicylic acid or derivatives of these acids; phosphorus compounds such as pyrophosphates; or enolates. Citric acid, tartaric acid, ascorbic acid, glucoheptonic acid or a derivative thereof are particularly suitable chelators for this purpose, because a chelate of technetium-99m with one of these chelators undergoes the desired ligand exchange particularly easily.

The most commonly used procedure for preparing [Tc^{v}O]⁺³N₂S₂ complexes is based on stannous (II) chloride reduction of [^{99m}Tc]pertechnetate, the common starting material. The labeling procedure normally relies on a Tc-99m ligand exchange reaction between Tc-99m (Sn)-glucoheptonate and the N₂S₂ ligand. Preparation of stannous (II) chloride and preserving it in a consistent stannous (II) form is critically important for the success of the labeling reaction. To stabilize the air-sensitive stannous ion it is a common practice in nuclear medicine to use a lyophilized kit, in which the stannous ion is in a lyophilized powder form mixed with an excess amount of glucoheptonate under an inert gas like nitrogen or argon. The preparation of the lyophilized stannous chloride/sodium glucoheptonate kits ensures that the labeling reaction is reproducible and predictable. The N₂S₂ ligands are usually air-sensitive (thiols are easily oxidized by air) and there are subsequent reactions which lead to decomposition of the ligands. The most convenient and predictable method to preserve the ligands is to produce lyophilized kits containing 100-500 μg of the ligands under argon or nitrogen.

The radiohalogenated compounds of this invention lend themselves easily to formation from materials which could be provided to users in kits. Kits for forming the imaging agents can contain, for example, a vial containing a physiologically suitable solution of an intermediate of Formula I or II in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and receptors in the brain imaged by means of measuring the gamma ray or photo emissions therefrom.

Since the radiopharmaceutical composition according to the present invention can be prepared easily and simply, the preparation can be carried out readily by the user. Therefore, the present invention also relates to a kit, comprising:

(1) A non-radiolabeled compound of the invention, the compound optionally being in a dry condition; and also optionally having an inert, pharmaceutically acceptable carrier and/or auxiliary substances added thereto; and

(2) a reducing agent and optionally a chelator;

wherein ingredients (1) and (2) may optionally be combined; and

further wherein instructions for use with a prescription for carrying out the above-described method by reacting ingredients (1) and (2) with technetium-99m in the form of a pertechnetate solution may be optionally included.

Examples of suitable reducing agents and chelators for the above kit have been listed above. The pertechnetate solution can be obtained by the user from a molybdenum-technetium generator. Such generators are available in a number of institutions that perform radiodiagnostic procedures. As noted above the ingredients (1) and (2) may be combined, provided they are compatible. Such a monocomponent kit, in which the combined ingredients are preferably lyophilized, is excellently suitable to be reacted by the user with the pertechnetate solution in a simple manner.

When desired, the radioactive diagnostic agent may contain any additive such as pH controlling agents (e.g., acids, bases, buffers), stabilizers (e.g., ascorbic acid) or isotonizing agents (e.g., sodium chloride).

Those skilled in the art are familiar with the various ways to detect labeled compounds for imaging purposes. For example, positron emission tomography (PET) or single photon emission computed tomography (SPECT) can be used to detect radiolabeled compounds. The label that is introduced into the compound can depend on the detection method desired. Those skilled in the art are familiar with PET detection of a positron-emitting atom, such as ¹⁸F. However, the present invention is also directed to specific compounds described herein where the ¹⁸F atom is replaced with a non-radiolabeled fluorine atom. Those skilled in the art are familiar with SPECT detection of a photon-emitting atom, such as ¹²³I or ^{99m}Tc. However, the present invention is also directed to specific compounds described herein where the ¹²³I atom is replaced with a non-radiolabeled iodine atom.

The radioactive diagnostic agent should have sufficient radioactivity and radioactivity concentration which can assure reliable diagnosis. The desired level of radioactivity can be attained by the methods provided herein for preparing compounds of Formula I, I′, II or II′. The imaging of amyloid deposits can also be carried out quantitatively so that the amount of amyloid deposits can be determined.

One of the key prerequisites for an in vivo imaging agent of the brain is the ability to cross the intact blood-brain barrier after a bolus iv injection. In the first step of the present method of imaging, a labeled compound of Formula I, I′, II or II′ is introduced into a tissue or a patient in a detectable quantity. The compound is typically part of a pharmaceutical composition and is administered to the tissue or the patient by methods well known to those skilled in the art.

For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray.

In a preferred embodiment of the invention, the labeled compound is introduced into a patient in a detectable quantity and after sufficient time has passed for the compound to become associated with amyloid deposits, the labeled compound is detected noninvasively inside the patient. In another embodiment of the invention, a labeled compound of Formula I, I′, II or II′ is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I is introduced into the tissue sample. After a sufficient amount of time for the compound to become bound to amyloid deposits, the compound is detected.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient.

Another aspect of the invention is a method of inhibiting amyloid plaque aggregation. For example, a compound of the present invention is tested in an established in-vitro immunoblot assay for its ability to inhibit the formation of AB oligomers and fibrils (Yang F, Liim G P, Begum A N et al. Curcumin inhibits formation of amyloid β oligomers and fibrils, binds plaques, and reduces amyloid in-vivo. J. Biol. Chem. 280:5892-5901, 2005). Curcumin, a natural molecule serves as positive control. Phen-napthalene and phen-quinoline compounds of the present invention are able to inhibit the aggregation of Aβ in a manner similar to Curcumin at concentrations of 1-100 μM.

The present invention also provides a method of inhibiting the aggregation of amyloid proteins to form amyloid deposits, by administering to a patient an amyloid inhibiting amount of a compound of Formula I, I′, II or II′.

The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

Those skilled in the art are readily able to determine an amyloid inhibiting amount by simply administering a compound of Formula I or II to a patient in increasing amounts until the growth of amyloid deposits is decreased or stopped. The rate of growth can be assessed using imaging as described above or by taking a tissue sample from a patient and observing the amyloid deposits therein.

The compounds of the present invention may be used in combination with one or more other drugs in the treatment, prevention, control, amelioration, or reduction of risk of diseases or conditions for which the compounds of the present invention have utility, where the combination of the drugs together are safer or more effective than either drug alone. Additionally, the compounds of the present invention may be used in combination with one or more other drugs that treat, prevent, control, ameliorate, or reduce the risk of side effects or toxicity of the compounds of the present invention. Such other drugs may be administered, by a route and in an amount commonly used therefore, contemporaneously or sequentially with the compounds of the present invention. Accordingly, the pharmaceutical compositions of the present invention include those that contain one or more other active ingredients, in addition to the compounds of the present invention. The combinations may be administered as part of a unit dosage form combination product, or as a kit or treatment protocol wherein one or more additional drugs are administered in separate dosage forms as part of a treatment regimen.

Examples of combinations of the compounds of the present invention with other drugs in either unit dose or kit form include combinations with: anti-Alzheimer's agents, for example beta-secretase inhibitors or gamma-secretase inhibitors; HMG-CoA reductase inhibitors; NSAIDs including ibuprofen; vitamin E; anti-amyloid antibodies, including humanized monoclonal antibodies; CB-1 receptor antagonists or CB-1 receptor inverse agonists; antibiotics such as doxycycline and rifampin; N-methyl-D-aspartate (NMDA) receptor antagonists, such as memantine; cholinesterase inhibitors such as galantamine, rivastigmine, donepezil and tacrine; growth hormone secretagogues such as ibutamoren, ibutamoren mesylate, and capromorelin; histamine H₃ antagonists; AMPA agonists; PDE IV inhibitors; GABAₐ inverse agonists; neuronal nicotinic agonists; or other drugs that affect receptors or enzymes that either increase the efficacy, safety, convenience, or reduce unwanted side effects or toxicity of the compounds of the present invention. The foregoing list of combinations is illustrative only and not intended to be limiting in any way.

The term “pharmaceutically acceptable salt” as used herein refers to those carboxylate salts or acid addition salts of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci. 66:1-19 (1977) which is incorporated herein by reference.)

The term “alkyl” as employed herein by itself or as part of another group refers to both straight and branched chain radicals of up to 4 carbons, preferably 1 or 2 carbons, more preferably 1 carbon (methyl).

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 4 carbon atoms in length, more preferably 1 or 2 carbon atoms in length.

The term “monoalkylamine” as employed herein by itself or as part of another group refers to an amino group which is substituted with one alkyl group as defined above. The term “dialkylamine” refers to an amino group which is substituted with two alkyl groups, which are defined above.

The term “halo” or “halogen” employed herein by itself or as part of another group refers to chlorine, bromine, fluorine or iodine, unless defined otherwise in specific uses in the text and/or claims.

The term “radiohalogen” employed herein by itself or as part of another group refers to ¹⁸F, ¹⁹F, ¹²³I, ¹²⁵I, ¹³¹I, ⁷⁶Br and ⁷⁷Br.

The term “halo(C₁₋₄)alkyl” as employed herein refers to any of the above alkyl groups substituted by one or more chlorine, bromine, fluorine or iodine with fluorine being preferred. Useful groups are chloromethyl, iodomethyl, trifluoromethyl, 2,2,2-trifluoroethyl, and 2-chloroethyl. Most preferably, the alkyl is substituted with a single halo, such as fluorine, at the distal end of the alkyl. The term “radiohalo(C₁₋₄)alkyl” refers to a halo(C₁₋₄)alkyl group as defined above that contains a halogen radioisotope. One example of this type of group is ¹⁸F—(C₁₋₄)alkyl-.

The term “hydroxyalkyl” as employed herein by itself or as part of another group refers to linear or branched alkyl groups containing an —OH substituent.

The term “aryl” as employed herein by itself or as part of another group refers to monocyclic or bicyclic aromatic groups containing from 5 to 14 atoms in the ring portion, preferably 6-10 carbons in the ring portion, such as phenyl, naphthyl or tetrahydronaphthyl. As employed herein, each aryl contains X or -Ch as a substituent. Preferable values under the scope of C₆₋₁₀ aryl include the following moieties, each of which contains X or -Ch as a substituent: phenyl, naphthyl and tetrahydronaphthyl. The aryl group can also contain a heteroatom, such as N, S or O to form a “heteroaryl.” Preferable values of under the scope of heteroaryl include: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl and phenoxazinyl groups.

The term “aryloxy” as employed herein refers to an “aryl” group bonded to an oxygen atom, and include benzyloxy and phenoxy and the like. Benzoyloxy refers to an ester.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formula I, I′, II or II′ into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

### EXAMPLES

### Experimental

All reagents used in the synthesis were commercial products and were used without further purification unless otherwise indicated. Microwave reactions were conducted using Biotage Initiated system. Preparative Thin Layer Chromatography (PTLC) was performed on Analtech Uniplate (20 cm×20 cm, 2000 microns). Flash column chromatography was performed on 230-400 mesh silica gel (Biotage Flash 40M). ¹H NMR spectra were obtained on Bruker spectrometers (Bruker DPX 200). Chemical shifts are reported as 6 values with respect to residual protons in CDCl₃ unless otherwise mentioned. Coupling constants are reported in Hz. The multiplicity is defined by s (singlet), d (doublet), t (triplet), br (broad), m (multiplet). Mass spectrometry was performed by the McMaster Regional Centre for Mass Spectrometry, McMaster University

### (6-bromonaphthalen-2-yloxy)(tert-butyl)dimethylsilane (7)

TBSCl (347 mg, 2.3 mmol) was added to a solution of 6-bromonaphthalen-2-ol (446 mg, 2.0 mmol) in dichloromethane (20 ml) followed by the addition of imidazole (272 mg, 4.0 mmol). The reaction mixture was stirred at room temperature for 2 h and then water was added. Organic layer was separated, washed with brine, dried over anhydrous sodium sulfate and evaporated to obtain crude product 7 (680 mg, 100%), which was pure enough and was used directly for next step. 7: ¹H NMR (CDCl₃): δ 7.91 (1H, m), 7.55 (3H, m), 7.13 (2H, m), 1.02 (9H, s), 0.25 (6H, s).

### 6-(tert-butyldimethylsilyloxy)naphthalen-2-ylboronic acid (8)

A solution of compound 7 (674 mg, 2 mmol) in anhydrous THF (15 ml) was cooled to −78° C. n-Butyl lithium (1.6 M, 1.88 ml) was added dropwise within 30 minutes and the reaction mixture was stirred at −78° C. for 20 min. Triisopropyl borate (1.13 g, 6 mmol) was then added and the reaction mixture was stirred at −78° C. for another 20 min and then allowed to warm up to room temperature. 1N HCl was added until water layer became acidic. Ethyl acetate was added and organic layer was separated, washed with brine, dried over anhydrous sodium sulfate and evaporated. Residue was applied on Biotage Flash column chromatography (1% methanol in dichloromethane as the eluant) to obtain product 8 (320 mg, 53%), ¹H NMR (CDCl₃): δ 8.75 (1H, m), 8.24 (1H, d, J=8.2 Hz), 7.95 (1H, d, J=8.8 Hz), 7.81 (1H, d, J=8.2 Hz), 7.16 (2H, m), 1.05 (9H, s), 0.27 (6H, s).

### 6-(4-nitrophenyl)naphthalen-2-ol (9)

Sodium carbonate (112 mg, 1.06 mmol) in water (5 ml) was added to a solution of 8 (320 mg, 1.06 mmol), para-iodonitrobenzene (264 mg, 1.06 mmol) and Pd(PPh₃)₄ (66 mg, 0.053 mmol) in toluene (10 ml). The reaction mixture was degassed by bubbling nitrogen for 10 min and then heated to 100° C. for 2 h. After cooling down to the room temperature, ethyl acetate and water was added. The organic layer was separated, washed with brine dried with anhydrous sodium sulfate and evaporated. The residue (470 mg) was dissolved in THF (20 ml) and TBAF (1M in THF, 6 ml) was added. After stirring at the room temperature for 3 h, the reaction mixture was poured in to water and extracted with ethyl acetate. Organic layer was washed with brine, dried with sodium sulfate and evaporated. The residue was purified by Biotage Flash column chromatography (10% hexane in dichloromethane as the eluant) to obtain product 9 (230 mg, Y: 81.8%) as an orange solid. ¹H NMR (CDCl₃): δ 8.25 (2H, d, J=7.0 Hz), 7.95 (1H, m), 7.75 (4H, M), 7.61 (1H, d, d, J₁=8.6 Hz, J₂=1.8 Hz), 7.11 (2H, m). HRMS (EI) m/z calcd. for [C₁₆H₁₁NO₃]⁺ 265.0739, found 265.0755

### 2-(2-(2-(2-fluoroethoxy)ethoxy)ethoxy)-6-(4-nitrophenyl)naphthalene (10a)

The mixture of compound 9 (90 mg, 0.34 mmol), 1-chloro-2-(2-(2-fluoroethoxy)ethoxy)ethane (70 mg, 0.41 mmol), potassium carbonate (140 mg, 1.0 mmol) and anhydrous DMF (5 ml) was put in a sealed microwavable vial (biotage) and subjected to microwave irradiation (Biotage Initiated system) at the following condition: 180° C., 10 min, high absorption level. After the reaction, water and ethyl acetate was added. Organic layer was separated, washed with brine, dried over anhydrous sodium sulfate and evaporated. The residue was purified by PTLC (100% dichloromethane as the developing solvent) to obtain product 10a (120 mg, Y: 89%). ¹H NMR (CDCl₃): δ 8.32 (2H, d, J=9.0 Hz), 8.02 (1H, m), 7.83 (4H, m), 7.70 (1H, d, d, J₁=8.6 Hz, J₂=1.8 Hz), 7.21 (2H, m), 4.57 (2H, d, t, J₁=49.6 Hz, J₂=4.2 Hz), 4.30 (2H, t, J=4.6 Hz), 3.96 (2H, t, J=4.8 Hz), 3.75 (6H, m). HRMS (EI) m/z calcd. for [C₂₂H₂₂FNO₅]⁺ 399.1482, found 399.1471.

### 2-(2-(2-(6-(4-nitrophenyl)naphthalen-2-yloxy)ethoxy)ethoxy)ethanol (10b)

The mixture of compound 9 (140 mg, 0.53 mmol), 2-(2-(2-chloroethoxy)ethoxy)ethanol (98.3 mg, 0.58 mmol), potassium carbonate (219 mg, 1.6 mmol) and anhydrous DMF (5 ml) was put in a sealed microwavable vial (biotage) and subjected to microwave irradiation (Biotage Initiated system) at the following condition: 160° C., 10 min, high absorption level. After the reaction, water and ethyl acetate was added. Organic layer was separated, washed with brine, dried over anhydrous sodium sulfate and evaporated. The residue was purified by PTLC (3% methanol in dichloromethane as the developing solvent) to obtain product 10b (170 mg, Y: 81%). ¹H NMR (CDCl₃): δ 8.32 (2H, d, J=9.0 Hz), 8.21 (1H, d, J=1.6 Hz), 7.83 (4H, m), 7.70 (1H, d, d, J₁=8.6 Hz, J₂=1.8 Hz), 7.21 (2H, m), 4.29 (2H, t, J=4.6 Hz), 3.96 (2H, t, J=4.6 Hz), 3.71 (8H, m), 2.37 (1H, t, J=5.8 Hz). HRMS (EI) m/z calcd. for [C₂₂H₂₃NO₆]⁺ 397.1525, found 397.1515

### 4-(6-(2-(2-(2-fluoroethoxy)ethoxy)ethoxy)naphthalen-2-yl)benzenamine (11a)

Concentrated HCl (0.5 ml) was added to a suspension of compound 10a (115 mg, 0.288 mmol) and stannous chloride (219 mg, 1.15 mmol) in ethanol (10 ml). The reaction mixture was heated to reflux for 2 h. After cooling down to the room temperature, reaction mixture was basified by 2N NaOH and extracted with ethyl acetate. Organic layer was separated, washed with brine, dried over anhydrous sodium sulfate and evaporated. The residue was purified by PTLC (2% methanol in dichloromethane as the developing solvent) to obtain product 11a (80 mg, Y: 76%). ¹H NMR (CDCl₃): δ 7.86 (1H, b), 7.73 (2H, d, J=8.6 Hz), 7.62 (1H, d, J=8.6 Hz), 7.53 (2H, d, J=8.4 Hz), 7.16 (2H, m), 6.89 (2H, d, J=8.4 Hz), 4.54 (2H, d, t, J₁=47.8 Hz, J₂=4.2 Hz), 4.25 (2H, t, J=4.6 Hz), 3.92 (2H, t, J=4.6 Hz), 3.76 (6H, m). HRMS (EI) m/z calcd. for [C₂₂H₂₄FNO₃]⁺ 369.1740, found 369.1750.

### 2-(2-(2-(6-(4-aminophenyl)naphthalen-2-yloxy)ethoxy)ethoxy)ethanol (11b)

Concentrated HCl (0.5 ml) was added to a suspension of compound 10b (160 mg, 0.40 mmol) and stannous chloride (303 mg, 1.6 mmol) in ethanol (10 ml). The reaction mixture was heated to reflux for 2 h. After cooling down to the room temperature, the reaction mixture was basified with 2N NaOH and extracted with ethyl acetate. Organic layer was separated, washed with brine, dried over anhydrous sodium sulfate and evaporated. The residue was purified by PTLC (3% methanol in dichloromethane as the developing solvent) to obtain product 11b (120 mg, Y: 81%). ¹H NMR (CDCl₃): δ 7.86 (1H, br), 7.73 (2H, d, J=8.6 Hz), 7.62 (1H, d, J=8.6 Hz), 7.53 (2H, d, J=8.4 Hz), 7.16 (2H, m), 6.89 (2H, d, J=8.4 Hz), 4.54 (2H, d, t, J₁=47.8 Hz, J₂=4.2 Hz), 4.25 (2H, t, J=4.6 Hz), 3.92 (2H, t, J=4.6 Hz), 3.76 (6H, m). HRMS (EI) m/z calcd. for [C₂₂H₂₅NO₄]⁺ 367.1784, found 367.1780.

### 4-(6-(2-(2-(2-fluoroethoxy)ethoxy)ethoxy)naphthalen-2-yl)-N,N-dimethylbenzenamine (12a)

Sodium cyanoborohydride (15.3 mg 0.24 mmol) was added to a solution of compound 11a (30 mg, 0.081 mmol), para-formaldehyde (24.4 mg, 0.81 mmol) in acetic acid (5 ml). The reaction mixture was stirred at room temperature overnight and poured onto ice. 2N NaOH was used to adjust pH to 10 and solution was extracted with ethyl acetate. Organic layer was separated, washed with brine, dried over anhydrous sodium sulfate and evaporated. The residue was purified by PTLC (1.5% methanol in dichloromethane as the developing solvent) to obtain product 12a (31 mg, Y: 96%). ¹H NMR (CDCl₃): δ 7.90 (1H, br), 7.75 (2H, d, J=8.8 Hz), 7.68 (1H, d, d, J₁=8.6 Hz, J₂=1.6 Hz), 7.62 (2H, d, J=8.8 Hz), 7.18 (2H, m), 6.88 (2H, d, J=8.0 Hz), 4.56 (2H, d, t, J₁=47.8 Hz, J₂=4.2 Hz), 4.27 (2H, t, J=4.6 Hz), 3.94 (2H, t, J=4.8 Hz), 3.76 (6H, m), 3.02 (6H, s). HRMS (EI) m/z calcd. for [C₂₄H₂₈FNO₃]⁺ 397.2053, found 397.2046.

### 4-(6-(2-(2-(2-fluoroethoxy)ethoxy)ethoxy)naphthalen-2-yl)-N-methylbenzenamine (13a)

Sodium methoxide (0.5 M in methanol, 1.2 ml, 0.6 mmol) was added to a solution of compound 11a (44 mg, 0.12 mmol) in methanol (8 ml), followed by the addition of para-formaldehyde (18 mg, 0.6 mmol). The reaction mixture was refluxed for 1.5 h and cooled to 0° C. Sodium borohydride (27 mg, 0.7 mmol) was added in caution and the reaction mixture was refluxed again for 1.5 h. After cooling down to 0° C., the reaction mixture was filtered to get crude solid, which was then purified by PTLC (2% methanol in dichloromethane as the developing solvent) to obtain 13a (33 mg, Y: 72%). ¹H NMR (CDCl₃): δ 7.89 (1H, br), 7.75 (2H, d, J=8.8 Hz), 7.67 (1H, d, d, J₁=8.6 Hz, J₂=1.6 Hz), 7.58 (2H, d, J=8.6 Hz), 7.18 (2H, m), 6.77 (2H, d, J=8.6 Hz), 4.56 (2H, d, t, J₁=47.8 Hz, J₂=4.2 Hz), 4.27 (2H, t, J=4.6 Hz), 3.95 (2H, t, J=4.8 Hz), 3.76 (6H, m), 2.91 (3H, s). HRMS (EI) m/z calcd. for [C₂₃H₂₆FNO₃]⁺ 383.1897, found 383.1881.

### 2-(2-(2-(6-(4-(methylamino)phenyl)naphthalen-2-yloxy)ethoxy)ethoxy)ethanol (13b)

Sodium methoxide (0.5 M in methanol, 3.0 ml, 1.5 mmol) was added to a solution of compound 11b (110 mg, 0.3 mmol) in methanol (8 ml), followed by the addition of para-formaldehyde (45 mg, 1.5 mmol). The reaction mixture was refluxed for 1.5 h and cooled to 0 C. Sodium borohydride (68 mg, 1.8 mmol) was added in caution and reaction mixture was refluxed again for 1.5 h. After cooling down to 0° C., the reaction mixture was partitioned between dichloromethane and water. Organic layer was separated, washed with brine, dried over anhydrous sodium sulfate and evaporated. The residue was purified by PTLC (4% methanol in dichloromethane as the developing solvent) to obtain product 13b (100 mg, Y: 88%). ¹H NMR (CDCl₃): δ 7.89 (1H, br), 7.75 (2H, d, J=8.4 Hz), 7.67 (1H, d, d, J₁=8.6 Hz, J₂=1.6 Hz), 7.56 (2H, d, J=8.6 Hz), 7.17 (2H, m), 6.72 (2H, d, J=8.6 Hz), 4.27 (2H, t, J=4.8 Hz), 3.94 (2H, t, J=4.8 Hz), 3.74 (6H, m), 3.63 (2H, m), 2.90 (3H, s). HRMS (EI) m/z calcd. for [C₂₃H₂₇NO₄]⁺ 381.1940, found 381.1946

### tert-butyl 4-(6-(2-(2-(2-hydroxyethoxy)ethoxy)ethoxy)naphthalen-2-yl)phenyl(methyl)carbamate (14b)

TBSCl (36.2 mg, 0.24 mmol) was added to a solution of 13b (76 mg, 0.2 mmol) in dichloromethane (10 ml) followed by the addition of imidazole (27.2 mg, 0.4 mmol). The reaction mixture was stirred at room temperature for 4 h. Water was added and organic layer was separated, washed with brine, dried over anhydrous sodium sulfate and evaporated. The residue was purified by PTLC (2% methanol in dichloromethane as the developing solvent) to obtain solid 74 mg (Y: 75%). A portion of this material (68 mg, 0.137 mmol) was dissolved in anhydrous THF (5 ml). Di-tert-butoxy dicarbonate (58.8 mg, 0.27 mmol) was added and the reaction mixture was refluxed overnight. Solvent was removed by vacuum and residue was purified by PTLC (2% methanol in dichloromethane as the developing solvent) to obtain solid 65 mg (Y: 79%). Total of this solid (65 mg, 0.11 mmol) was dissolved in THF (3 ml). Tetrabutyl ammonium fluoride (1M in THF, 0.55 ml) was added and the reaction mixture was stirred at room temperature for 3 h. Solvent was removed and residue was purified by PTLC (3 methanol in dichloromethane as the developing solvent) to obtain product 14b (51 mg, Y: 57% for three steps, based on 13b). ¹H NMR (CDCl₃): δ 7.94 (1H, br), 7.78 (2H, d, J=8.6 Hz), 7.66 (3H, m), 7.33 (2H, d, J=8.6 Hz), 7.2 (2H, m), 4.28 (2H, t, J=4.8 Hz), 3.95 (2H, t, J=4.8 Hz), 3.75 (6H, m), 3.63 (2H, m), 3.31 (3H, s), 1.49 (9H, s). HRMS (EI) m/z calcd. for [C₂₈H₃₅NO₆]⁺ 481.2464, found 481.2462.

### 2-(2-(2-(6-(4-(tert-butoxycarbonyl(methyl)amino)phenyl)naphthalen-2-yloxy)ethoxy)ethoxy)ethyl methanesulfonate (14c)

Methanesulfonyl chloride (63 mg, 0.55 mmol) was added to a solution of compound 14b (44 mg, 0.091 mmol) in dichloromethane (5 ml), followed by the addition of triethyl amine (90 mg, 0.91 mmol). The reaction mixture was stirred at room temperature overnight. Solvent was removed by vacuum and the residue was purified by PTLC (2% methanol in dichloromethane as the developing solvent) to obtain product 8c (46 mg, Y: 90%). ¹H NMR (CDCl₃): δ 7.95 (1H, br), 7.78 (2H, d, J=8.6 Hz), 7.66 (3H, m), 7.34 (2H, d, J=8.6 Hz), 7.20 (2H, m), 4.38 (2H, m), 4.27 (2H, t, J=4.7 Hz), 3.95 (2H, t, J=4.7 Hz), 3.75 (6H, m), 3.32 (3H, s), 3.04 (3H, s), 1.49 (9H, s). HRMS (EI) m/z calcd. for [C₂₉H₃₇NO₈S]⁺ 559.2240, found 559.2234.

### 6-bromonaphthalen-2-amine (15)

6-Bromonaphthalen-2-ol (1.5 g, 6.7 mmol) was heated with ammonium hydroxide (10 ml) and ammonium sulfite (3.5 g, 26 mmol) in a seal tube at 150° C. for 48 h. After cooling to room temperature, ethyl acetate was added and organic layer was separated, washed with brine, dried over anhydrous sodium sulfate and evaporated to obtain crude product 15 (1.4 g, Y; 94%), which was pure enough and can be used directly for next step without further purification. ¹H NMR (CDCl₃): δ 7.84 (1H, br), 7.56 (1H, d, J=9.6 Hz), 7.43 (2H, m), 6.95 (2H, m), 3.87 (2H, b). Leigh C. Anderson and Donald G. Thomas: Quinoidation of Triaryl Compounds—Hydroxynaphthyldiphenylcarbinols J. Am. Chem. Soc.; 65; 1943; 239, 241.

### 4-(6-aminonaphthalen-2-yl)phenol (16)

Palladium tetrakis(triphenylphosphine) (34.7 mg, 0.03 mmol) was added to a solution of compound 15 (200 mg, 0.9 mmol) and 4-hydroxyphenylboronic acid (165.5 mg, 1.2 mmol) in a mixed solvent of toluene (15 ml) and ethanol (5 ml), followed by the addition of tetrabutyl ammonium bromide (19 mg, 0.06 mmol) and sodium carbonate (2M aq., 4.0 ml). The solution was degassed by bubbling nitrogen for 10 min and then heated at 100° C. overnight. After cooling down to room temperature, the mixture was partitioned between ethyl acetate and water. Organic layer was separated, washed with brine, dried over sodium sulfate and concentrated to about 5 ml using vacuum. Precipitate was filtered out and washed with cold methanol to obtain product 16 (151 mg, Y: 71%) as a pale yellow solid, which was already pure enough and was used directly in next step without further purification. 16: ¹H NMR (DMSO-d⁶): δ 9.45 (1H, br), 7.80 (1H, b), 7.62 (1H, d, J=8.8 Hz), 7.53 (4H, m), 6.93 (1H, d, d, J₁=8.8 Hz, J₂=2.0 Hz), 6.82 (3H, m), 5.35 (2H, b). HRMS (EI) m/z calcd. for [C₁₆H₁₃NO]⁺ 235.0097, found 235.0091.

### 6-(4-(2-(2-(2-fluoroethoxy)ethoxy)ethoxy)phenyl)naphthalen-2-amine (17a)

The mixture of compound 16 (60 mg, 0.26 mmol), 1-chloro-2-(2-(2-fluoroethoxy)ethoxy)ethane (30) (52 mg, 0.30 mmol), potassium carbonate (106 mg, 0.8 mmol) and anhydrous DMF (5 ml) was put in a sealed microwavable vial (Biotage) and subjected to microwave irradiation (Biotage Initiated system) at the following condition: 180° C., 10 min, high absorption level. After the reaction, water and ethyl acetate was added. Organic layer was separated, washed with brine, dried over anhydrous sodium sulfate and evaporated. The residue was purified by PTLC (2% methanol in dichloromethane as the developing solvent) to obtain product 17a (68 mg, Y: 72%). ¹H NMR (CDCl₃): δ 7.85 (1H, s), 7.69 (2H, d, J=8.6 Hz), 7.60 (3H, m), 7.00 (4H, m), 4.57 (2H, d, t, Jⱼ=47.6 Hz, J₂=4.2 Hz), 4.20 (2H, t, J=4.8 Hz), 3.80 (10H, m). HRMS (EI) m/z calcd. for [C₂₂H₂₄FNO₃]⁺ 369.1740, found 369.1737.

### 2-(2-(2-(4-(6-aminonaphthalen-2-yl)phenoxy)ethoxy)ethoxy)ethanol (17b)

The mixture of compound 16 (70 mg, 0.30 mmol), 2-(2-(2-chloroethoxy)ethoxy)ethanol (67 mg, 0.36 mmol), potassium carbonate (124 mg, 0.9 mmol) and anhydrous DMF (5 ml) was put in a sealed microwavable vial (biotage) and subjected to microwave irradiation (Biotage Initiated system) at the following condition: 180° C., 10 min, high absorption level. After the reaction, water and ethyl acetate was added. Organic layer was separated, washed with brine, dried over anhydrous sodium sulfate and evaporated. The residue was purified by PTLC (3.5% methanol in dichloromethane as the developing solvent) to obtain product 17b (80 mg, Y: 73%). ¹H NMR (CDCl₃): δ 7.80 (1H, s), 7.65 (2H, d, J=8.6 Hz), 7.58 (3H, m), 6.94 (4H, m), 4.16 (2H, t, J=4.7 Hz), 3.86 (2H, t, J=4.7 Hz), 3.68 (6H, m), 3.58 (2H, m). HRMS (EI) m/z calcd. for [C₂₂H₂₅NO₄]⁺ 367.1784, found 367.1772.

### 6-(4-(2-(2-(2-fluoroethoxy)ethoxy)ethoxy)phenyl)-N,N-dimethylnaphthalen-2-amine (18a)

Sodium cyanoborohydride (15.0 mg 0.24 mmol) was added to a solution of compound 17a (29 mg, 0.08 mmol), para-formaldehyde (24 mg, 0.8 mmol) in acetic acid (5 ml). The reaction mixture was stirred at room temperature overnight and pour onto ice. 2N NaOH was used to adjust pH to 10 and solution was extracted with ethyl acetate. Organic layer was separated, washed with brine, dried over anhydrous sodium sulfate and evaporated. The residue was purified by PTLC (1.5% methanol in dichloromethane as the developing solvent) to obtain product 18a (15 mg, Y: 48%). ¹H NMR (CDCl₃): δ 7.85 (1H, br), 7.72 (2H, m), 7.61 (3H, m), 7.18 (1H, d, d, J₁=9.0 Hz, J₂=2.4 Hz), 7.01 (2H, d, J=8.8 Hz), 6.94 (1H, d, J=2.4 Hz), 4.57 (2H, d, t, Jⱼ=47.6 Hz, J₂=4.2 Hz), 4.20 (2H, t, J=4.8 Hz), 3.80 (8H, m), 3.06 (6H, s). HRMS (EI) m/z calcd. for [C₂₄H₂₈FNO₃]⁺ 397.2053, found 397.2039.

### 2-(2-(2-(4-(6-(dimethylamino)naphthalen-2-yl)phenoxy)ethoxy)ethoxy)ethanol (18 b)

Sodium cyanoborohydride (13 mg 0.21 mmol) was added to a solution of compound 17b (26 mg, 0.07 mmol), para-formaldehyde (21 mg, 0.7 mmol) in acetic acid (3 ml). The reaction mixture was stirred at room temperature overnight and pour onto ice. 2N NaOH was used to adjust pH to 10 and solution was extracted with ethyl acetate. Organic layer was separated, washed with brine, dried over anhydrous sodium sulfate and evaporated. The residue was purified by PTLC (4% methanol in dichloromethane as the developing solvent) to obtain product 18b (14 mg, Y: 50%). ¹H NMR (CDCl₃): δ 7.85 (1H, br), 7.72 (2H, m), 7.60 (3H, m), 7.18 (1H, d, d, J₁=9.0 Hz, J₂=2.4 Hz), 7.01 (2H, d, J=8.8 Hz), 6.94 (1H, d, J=2.4 Hz), 4.20 (2H, t, J=4.8 Hz), 3.90 (2H, t, J=4.8 Hz), 3.73 (6H, m), 3.63 (2H, m), 3.06 (6H, s). HRMS (EI) m/z calcd. for [C₂₄H₂₉NO₄]⁺ 395.2097, found 395.2083.

### 6-(4-(2-(2-(2-fluoroethoxy)ethoxy)ethoxy)phenyl)-N-methylnaphthalen-2-amine (19a)

Sodium methoxide (0.5 M in methanol, 0.94 ml, 0.47 mmol) was added to a solution of compound 17a (34.4 mg, 0.094 mmol) in methanol (5 ml), followed by the addition of para-formaldehyde (14 mg, 0.47 mmol). The reaction mixture was refluxed for 1.5 h and cooled to 0° C. Sodium borohydride (21.3 mg, 0.56 mmol) was added in caution and reaction mixture was refluxed again for 1.5 h. After cooling down to 0° C., the reaction mixture was filtered to get crude solid, which was then purified by PTLC (2% methanol in dichloromethane as the developing solvent) to obtain product 19a (30 mg, Y: 84%). ¹H NMR (CDCl₃): δ 7.83 (1H, br), 7.63 (5H, m), 7.01 (2H, d, J=8.8 Hz), 6.90 (1H, d, d, J₁=8.8 Hz, J₂=2.4 Hz), 6.81 (1H, m), 4.56 (2H, d, t, J₁=47.8 Hz, J₂=4.2 Hz), 4.20 (2H, t, J=4.6 Hz), 3.82 (9H, m), 2.95 (3H, d, J=5.2 Hz). HRMS (EI) m/z calcd. for [C₂₃H₂₆FNO₃]⁺ 383.1897, found 383.1900.

### 2-(2-(2-(4-(6-(methylamino)naphthalen-2-yl)phenoxy)ethoxy)ethoxy)ethanol (19b)

Sodium methoxide (0.5 M in methanol, 1.0 ml, 0.5 mmol) was added to a solution of compound 17b (35 mg, 0.095 mmol) in methanol (5 ml), followed by the addition of para-formaldehyde (14.4 mg, 0.5 mmol). The reaction mixture was refluxed for 1.5 h and cooled to 0° C. Sodium borohydride (21.6 mg, 0.57 mmol) was added in caution and reaction mixture was refluxed again for 1.5 h. After cooled down to 0° C., the reaction mixture was filtered to get crude solid, which was then purified by PTLC (4% methanol in dichloromethane as the developing solvent) to obtain product 19b (34.3 mg, Y: 94%). ¹H NMR (CDCl₃): δ 7.80 (1H, br), 7.60 (5H, m), 6.98 (2H, d, J=8.6 Hz), 6.88 (1H, d, d, J₁=8.8 Hz, J₂=2.4 Hz), 6.80 (1H, m), 4.18 (2H, t, J=4.7 Hz), 3.87 (2H, t, J=4.7 Hz), 3.70 (6H, m), 3.60 (2H, m), 2.92 (3H, s). HRMS (EI) m/z calcd. for [C₂₃H₂₇NO₄]⁺ 381.1940, found 381.1935.

### tert-butyl 6-(4-(2-(2-(2-hydroxyethoxy)ethoxy)ethoxy)phenyl)naphthalen-2-yl(methyl)carbamate (20b)

TBSCl (40.8 mg, 0.27 mmol) was added to a solution of 19b (86 mg, 0.23 mmol) in dichloromethane (10 ml) followed by the addition of imidazole (30.7 mg, 0.45 mmol). The reaction mixture was stirred at room temperature for 4 h. Water was added and organic layer was separated, washed with brine, dried over anhydrous sodium sulfate and evaporated. The residue was purified by PTLC (30% Ethyl acetate in Hexane) to obtain solid 90 mg (Y: 80.5%). A portion of this material (88 mg, 0.177 mmol) was dissolved in anhydrous THF (8 ml). Di-tert-butoxy dicarbonate (77.5 mg, 0.35 mmol) was added and the reaction mixture was refluxed overnight. Solvent was removed by vacuum and residue was purified by PTLC (25% Ethyl acetate in Hexane) to obtain solid 90 mg (Y: 85%). Total of this solid (90 mg, 0.15 mmol) was dissolved in THF (5 ml). Tetrabutyl ammonium fluoride (1M in THF, 0.77 ml) was added and the reaction mixture was stirred at room temperature for 3 h. Solvent was removed and residue was purified by PTLC (3% methanol in dichloromethane as the developing solvent) to obtain product 20b (70 mg, Y: 66.4% for three steps, based on 19b). ¹H NMR (CDCl₃): δ 7.95 (1H, br), 7.82 (2H, d, d, J₁=8.6 Hz, J₂=3.0 Hz), 7.66 (4H, m), 7.43 (1H, d, d, J₁=8.8 Hz, J₂=2.0 Hz), 7.04 (2H, d, J=8.8 Hz), 4.21 (2H, t, J=4.8 Hz), 3.91 (2H, t, J=4.8 Hz), 3.75 (6H, m), 3.63 (2H, m), 3.37 (3H, s), 1.47 (9H, s). HRMS (EI) m/z calcd. for [C₂₈H₃₅NO₆]⁺ 481.2464, found 481.2460.

### 2-(2-(2-(4-(6-(tert-butoxycarbonyl(methyl)amino)naphthalen-2-yl)phenoxy)ethoxy)ethoxy)ethyl methanesulfonate (20c)

Methanesulfonyl chloride (46.4 mg, 0.41 mmol) was added to a solution of compound 20b (65 mg, 0.14 mmol) in dichloromethane (5 ml), followed by the addition of triethyl amine (54 mg, 0.54 mmol). The reaction mixture was stirred at room temperature overnight. Solvent was removed by vacuum and the residue was purified by PTLC (3% methanol in dichloromethane as the developing solvent) to obtain product 20c (72 mg, Y: 95%). ¹H NMR (CDCl₃): δ 7.95 (1H, br), 7.82 (2H, d, d, J₁=8.6 Hz, J₂=3.0 Hz), 7.66 (4H, m), 7.42 (1H, d, d, J₁=8.8 Hz, J₂=2.0 Hz), 7.02 (2H, d, J=8.6 Hz), 4.39 (2H, m), 4.20 (2H, t, J=4.8 Hz), 3.88 (2H, t, J=4.7 Hz), 3.76 (6H, m), 3.37 (3H, s), 3.06 (3H, s), 1.47 (9H, s). HRMS (EI) m/z calcd. for [C₂₉H₃₇NO₈S]⁺ 559.2240, found 559.2214.

### 6-bromo-N,N-dimethylnaphthalen-2-amine (21)

Sodium cyanoborohydride (170 mg 2.7 mmol) was added to a solution of compound 15 (200 mg, 0.9 mmol), para-formaldehyde (270 mg, 9.0 mmol) in acetic acid (5 ml). The reaction mixture was stirred at room temperature overnight and pour onto ice. 2N NaOH was used to adjust pH to 10 and solution was extracted with ethyl acetate. Organic layer was separated, washed with brine, dried over anhydrous sodium sulfate and evaporated. The residue was purified by PTLC (1.5% methanol in dichloromethane as the developing solvent) to obtain product 21 (120 mg, Y: 58%). ¹H NMR (CDCl₃): δ 7.82 (1H, d, J=2.0 Hz), 7.60 (1H, d, J=9.0 Hz), 7.52 (1H, d, J=8.8 Hz), 7.44 (1H, d, d, Jⱼ=8.8 Hz, J₂=2.0 Hz), 7.16 (1H, d, d, J₁=9.0 Hz, J₂=2.6 Hz), 6.86 (1H, d, J=2.6 Hz), 3.04 (6H, s).

### 2-bromo-6-(2-(2-(2-fluoroethoxy)ethoxy)ethoxy)naphthalene (22)

The mixture of compound 2-hydroxy-6-bromo-naphthalene (450 mg, 2.0 mmol), 1-chloro-2-(2-(2-fluoroethoxy)ethoxy)ethane (30) (378.3 mg, 2.2 mmol), potassium carbonate (828 mg, 6.0 mmol) and anhydrous DMF (5 ml) was put in a sealed microwavable vial (biotage) and subjected to microwave irradiation (Biotage Initiated system) at the following condition: 180° C., 10 min, high absorption level. After the reaction, water and ethyl acetate was added. Organic layer was separated, washed with brine, dried over anhydrous sodium sulfate and evaporated. The residue was purified by PTLC (15% ethyl acetate in hexane as the developing solvent) to obtain product 22 (440 mg, Y: 61%). ¹H NMR (CDCl₃): δ 7.91 (1H, br), 7.61 (2H, m), 7.49 (1H, d, d, J₁=8.8 Hz, J₂=2.0 Hz), 7.19 (1H, d, d, J₁=9.0 Hz, J₂=2.4 Hz), 7.10 (1H, d, J=2.4 Hz), 4.56 (2H, d, t, J₁=47.8 Hz, J₂=4.2 Hz), 4.25 (2H, t, J=4.6 Hz), 3.93 (2H, t, J=4.8 Hz), 3.76 (6H, m).

### 6-(4-methoxyphenyl)-N,N-dimethylnaphthalen-2-amine (23)

Palladium tetrakis(triphenylphosphine) (12 mg, 0.011 mmol) was added to a solution of compound 21 (53 mg, 0.21 mmol), 4-methoxyphenylboronic acid (32 mg, 0.21 mmol) in DME (4 ml). The solution was degassed by bubbling nitrogen for 10 min. A pre-degassed solution of sodium carbonate (2M, 2.0 ml) was then added. Under the nitrogen atmosphere, the reaction mixture was heated at 100° C. overnight. The mixture was then cooled down to room temperature. Ethyl acetate and water was added. Organic layer was separated, washed with brine, dried over sodium sulfate and evaporated. The residue was purified by PTLC (30% Hexane in dichloromethaneas the developing solvent) to obtain product 23 (151 mg, Y: 51%). ¹H NMR (CDCl₃): δ 7.84 (1H, br), 7.72 (2H, m), 7.62 (3H, m), 7.18 (1H, d, d, J₁=9.0 Hz, J₂=2.4 Hz), 7.00 (2H, d, J=9.0 Hz), 6.94 (1H, d, J=2.4 Hz), 3.87 (3H, s), 3.06 (6H, s). HRMS (EI) m/z calcd. for [C₁₉H₁₉NO]⁺ 277.1467, found 277.1455.

### 4-(6-(dimethylamino)naphthalen-2-yl)phenol (24)

Palladium tetrakis(triphenylphosphine) (11.6 mg, 0.01 mmol) was added to a solution of compound 21 (50 mg, 0.2 mmol), 4-hydroxyphenylboronic acid (30.3 mg, 0.22 mmol) in Toluene (4 ml) and Ethanol (2 ml). The solution was degassed by bubbling nitrogen for 10 min. A pre-degassed solution of sodium carbonate (2M, aq, 1 ml) was then added. Under the nitrogen atmosphere, the reaction mixture was heated at 100° C. overnight. After cooling down to the room temperature, the mixture was added ethyl acetate and water. Organic layer was separated, washed with brine, dried over anhydrous sodium sulfate and evaporated. The residue was purified by PTLC (20% ethyl acetate in hexane as the developing solvent) to obtain product 24 (18 mg, Y: 34%). ¹H NMR (CDCl₃): δ 7.78 (1H, br), 7.67 (2H, m), 7.53 (3H, m), 7.14 (1H, d, d, Jⱼ=9.0 Hz, J₂=2.6 Hz), 6.87 (3H, m), 3.00 (6H, s). HRMS (EI) m/z calcd. for [C₁₈H₁₇NO]⁺ 263.1310, found 263.1299.

### 6-(4-methoxyphenyl)naphthalen-2-ol (25)

Palladium tetrakis(triphenylphosphine) (23 mg, 0.02 mmol) was added to a solution of 6-bromonaphthalene-2-ol (223 mg, 1.0 mmol), 4-methoxyphenylboronic acid (152 mg, 1.0 mmol) in DME (10 ml). The solution was degassed by bubbling nitrogen for 10 min. A pre-degassed solution of sodium carbonate (2M, aq, 5 ml) was then added. Under the nitrogen atmosphere, the reaction mixture was heated at 100° C. overnight. The mixture was then cooled down to room temperature. Ethyl acetate and water was added. Organic layer was separated, washed with brine, dried over sodium sulfate and evaporated. The residue was purified by PTLC (15% Ethyl acetate in Hexane as the developing solvent) to obtain product 25 (178 mg, Y: 71%). ¹H NMR (CDCl₃): δ 7.87 (1H, br), 7.71 (2H, m), 7.60 (3H, m), 7.10 (2H, m), 6.98 (2H, d, J=8.8 Hz), 3.84 (3H, s). HRMS (EI) m/z calcd. for [C₁₇H₁₄O₂]⁺ 250.0994, found 250.0989.

### 4-(6-(2-(2-(2-fluoroethoxy)ethoxy)ethoxy)naphthalen-2-yl)phenol (26)

Palladium tetrakis(triphenylphosphine) (11.6 mg, 0.01 mmol) was added to a solution of compound 22 (71 mg, 0.2 mmol), 4-hydroxyphenylboronic acid (30.3 mg, 0.22 mmol) in Toluene (5 ml) and Ethanol (2 ml). The solution was degassed by bubbling nitrogen for 10 min. A pre-degassed solution of sodium carbonate (2M, aq, 1 ml) was then added. Under the nitrogen atmosphere, the reaction mixture was heated at 100° C. overnight. The mixture was then cooled down to room temperature. Ethyl acetate and water was added. Organic layer was separated, washed with brine, dried over sodium sulfate and evaporated. The residue was purified by PTLC (2% methanol in dichloromethane as the developing solvent) to obtain product 26 (30 mg, Y: 40.6%). ¹H NMR (CDCl₃): δ 7.85 (1H, br), 7.71 (2H, d, J=8.4 Hz), 7.62 (1H, d, d, J₁=8.4 Hz, J₂=2.0 Hz), 7.52 (2H, d, t, Jⱼ=8.6 Hz, J₂=2.0 Hz), 7.14 (2H, m), 6.90 (2H, d, t, J₁=8.6 Hz, J₂=2.0 Hz), 4.53 (2H, d, t, J₁=47.8 Hz, J₂=4.2 Hz), 4.23 (2H, t, J=4.8 Hz), 3.91 (2H, t, J=4.8 Hz), 3.74 (6H, m). HRMS (EI) m/z calcd. for [C₂₂H₂₃FO₄]⁺ 370.1580, found 370.1570.

### 1-(2-(2-(2-fluoroethoxy)ethoxy)ethoxy)-4-iodobenzene (27)

The mixture of para-iodophenol (440 mg, 2.0 mmol), 1-chloro-2-(2-(2-fluoroethoxy)ethoxy)ethane (30) (409 mg, 2.4 mmol), potassium carbonate (552 mg, 4.0 mmol) and anhydrous DMF (5 ml) was put in a sealed microwavable vial (biotage) and subjected to microwave irradiation (Biotage Initiated system) at the following condition: 180° C., 10 min, high absorption level. After the reaction, water and ethyl acetate was added. Organic layer was separated, washed with brine, dried over anhydrous sodium sulfate and evaporated. The residue was purified by PTLC (10% ethyl acetate in hexane as the developing solvent) to obtain product 27 (600 mg, Y: 84.7%). ¹H NMR (CDCl₃): δ 7.54 (2H, d, J=9.0 Hz), 6.69 (2H, d, J=9.0 Hz), 4.56 (2H, d, t, J₁=47.8 Hz, J₂=4.2 Hz), 4.09 (2H, t, J=4.8 Hz), 3.85 (3H, m), 3.72 (5H, m). HRMS (EI) m/z calcd. for [C₁₂H₁₆FIO₃]⁺ 354.0128, found 354.0121.

### 6-(4-(2-(2-(2-fluoroethoxy)ethoxy)ethoxy)phenyl)naphthalen-2-ol (28)

Palladium tetrakis(triphenylphosphine) (11.6 mg, 0.01 mmol) was added to a solution of compound 27 (71 mg, 0.2 mmol), 2 (60 mg, 0.2 mmol) and tetrabutylammonium bromide (5 mg, 0.015 mmol) in Toluene (5 ml) and Ethanol (2 ml). The solution was degassed by bubbling nitrogen for 10 min. A pre-degassed solution of sodium carbonate (2M, aq, 1 ml) was then added. Under the nitrogen atmosphere, the reaction mixture was heated at 100° C. overnight. The mixture was then cooled down to room temperature. Ethyl acetate and water was added. Organic layer was separated, washed with brine, dried over sodium sulfate and evaporated. The residue was dissolved in THF (4 ml), TBAF (1M in THF, 1 ml) was added slowly. Reaction mixture was stirred at room temperature for 3 h. Solvent was removed by vacuum. Residue was purified by PTLC (45% ethyl acetate in hexane as the developing solvent) to obtain product 28 (50.7 mg, Y: 68.5%). ¹H NMR (CDCl₃): δ 7.88 (1H, br), 7.73 (2H, m), 7.60 (3H, m), 7.12 (2H, m), 6.97 (2H, d, J=8.8 Hz), 5.34 (1H, br), 4.58 (2H, d, t, J₁=47.8 Hz, J₂=4.2 Hz), 4.17 (2H, t, J=4.8 Hz), 3.83 (8H, m). HRMS (EI) m/z calcd. for [C₂₂H₂₃FO₄]⁺ 370.1580, found 370.1574.

### Methanesulfonic acid 2-[2-(2-chloro-ethoxy)-ethoxy]-ethyl ester (29)

Methanesulfonyl chloride (40.8 g 0.356 mol) was added slowly to a solution of 2-(2-(2-chloroethoxy)ethoxy)ethanol (30 g, 0.178 mol) and triethyl amine (54 g, 0.534 mol) in dichloromethane (250 ml) at 0° C. The solution was then raised to room temperature and stirred for 4.5 hour. Reaction mixture was transferred into a separatory funnel. Organic layer was washed with water (150 ml×2) then brine (150 ml) and dried over sodium sulfate. After removing the solvent under vacuum, crude product 29 was obtained as reddish oil (44 g, 100%). Without purification, the crude product can be used directly for next step. ¹H NMR 6 4.37 (t, 2H), 3.67 (m, 10H), 3.07 (s, 3H)

### 1-Chloro-2-[2-(2-fluoro-ethoxy)-ethoxy]-ethane (30)

A solution of anhydrous TBAF^{c} (85 g 0.33 mol) in anhydrous THF (250 ml) was added to a solution of compound 29 (36 g, 0.15 mol) in anhydrous THF (150 ml). The mixture was stirred at 60° C. for 2 hour and cooled to room temperature. THF was removed under vacuum at room temperature. Dichloromethane (300 ml) was added to the residue and organic layer was washed with water (300 ml×2) and brine (150 ml) and then dried over sodium sulfate. Solvent was removed under low vacuum (−20 mmHg) and then high vacuum was applied and product 30 was distilled out at 50-55° C., 0.4 mmHg as a clear liquid (15.8 g, 63%): ¹H NMR 6 4.55 (d, t, 2H, J₁=47 Hz, J₂=4.0 Hz), 3.74 (m, 10H).

### 6-bromo-2-(2-(2-(2-fluoroethoxy)ethoxy)ethoxy)quinoline (31a)

Potassium tert-butoxide (177 mg, 1.58 mmol) was added to a solution of 2-(2-(2-fluoroethoxy)ethoxy)ethanol^{b} (120 mg, 0.79 mmol) in acetonitrile (10 ml). The solution was cooled to 0° C. and 6-bromo-2-hydroxy-quinoline (191.4 mg, 0.79 mmol) was added in portions. Reaction mixture was heated to 80° C. for 1 h and cooled down. Dichloromethane and water was added. Organic layer was separated, washed with brine, dried over anhydrous sodium sulfate and evaporated. The residue was purified by Biotage medium pressure column chromatography (15% Ethyl acetate in Hexane as the eluant) to obtain product 31a (210 mg, Y: 74%). ¹H NMR (CDCl₃): δ 7.87 (2H, m), 7.67 (2H, m), 6.95 (1H, d, J=8.8 Hz), 4.65 (3H, m), 4.43 (1H, m), 3.92 (2H, t, J=4.8 Hz), 3.76 (6H, m). HRMS (EI) m/z calcd. for [C₁₅H₁₇BrFNO₃]⁺ 357.0376, found 357.0374.

### 2-(2-(2-(6-bromoquinolin-2-yloxy)ethoxy)ethoxy)ethanol (31b)

Potassium tert-butoxide (138 mg, 1.23 mmol) was added to a solution of triethyleneglycol (616 mg, 4.1 mmol) in acetonitrile (10 ml). The solution was cooled to 0° C. and 6-bromo-2-hydroxy-quinolin (100 mg, 0.41 mmol) was added in portions. Reaction mixture was heated to 80° C. for 1 h and cooled down. Dichloromethane and water was added. Organic layer was separated, washed with brine, dried over anhydrous sodium sulfate and evaporated. The residue was purified by PTLC (3.5% methanol in dichloromethane as the developing solvent) to obtain product 31b (134 mg, Y: 91%). ¹H NMR (CDCl₃): δ 7.86 (2H, m), 7.67 (2H, m), 6.95 (1H, d, J=8.8 Hz), 4.64 (2H, t, J=4.8 Hz), 3.91 (2H, t, J=4.8 Hz), 3.72 (6H, m), 3.61 (2H, m), 2.48 (1H, b). HRMS (EI) m/z calcd. for [C₁₅H₁₈BrNa]⁺ 355.0419, found 355.0402.

### 4-(2-(2-(2-(2-fluoroethoxy)ethoxy)ethoxy)quinolin-6-yl)-N,N-dimethylbenzenamine (32a)

Compound 31a (60 mg, 0.168 mmol), 4-(dimethylamino)phenylboronic acid (33 mg, 0.2 mmol), Palladium tetrakistriphenylphosphin (9.7 mg, 0.0084 mmol) and sodium carbonate (2M aq. 0.42 ml, 0.84 mmol) was added to DME (5 ml). The reaction mixture was degassed by bubbling nitrogen for 10 min and then heated to 90° C. overnight. After cooling down to room temperature, the mixture was partitioned between ethyl acetate and water. The organic layer was separated, washed with brine, dried over anhydrous sodium sulfate and evaporated. The residue was purified by PTLC twice (30% ethyl acetate in hexane as the developing solvent) to obtain product 32a (20 mg, Y: 30%). ¹H NMR (CDCl₃): δ 7.99 (1H, d, J=8.8 Hz), 7.84 (3H, b), 7.60 (2H, d, J=8.8 Hz), 6.94 (1H, d, J=8.8 Hz), 6.85 (2H, d J=8.8 Hz), 4.66 (3H, m), 4.44 (1H, m), 3.93 (2H, t, J=4.8 Hz), 3.76 (6H, m), 3.02 (6H, s). HRMS (EI) m/z calcd. for [C₂₃H₂₇FN₂O₃]⁺ 398.2006, found 398.2005.

### 2-(2-(2-(6-(4-(dimethylamino)phenyl)quinolin-2-yloxy)ethoxy)ethoxy)ethanol (32b)

Compound 31b (100 mg, 0.28 mmol), 4-(dimethylamino)phenylboronic acid (56 mg, 0.34 mmol), Palladium tetrakistriphenylphosphin (16 mg, 0.014 mmol) and sodium carbonate (2M aq. 0.7 ml, 1.4 mmol), tetra-butylammonium bromide (13.5 mg, 0.042 mmol) was added to a mixed solvent of toluene (8 ml) and ethanol (2 ml). The reaction mixture was degassed by bubbling nitrogen for 10 min and then heated to 90° C. overnight. After cooled down to room temperature, the mixture was partitioned between ethyl acetate and water. The organic layer was separated, washed with brine, dried over anhydrous sodium sulfate and evaporated. The residue was purified by PTLC (pure ethyl acetate as the developing solvent) to obtain product 32b (105 mg, Y: 94%). ¹H NMR (CDCl₃): δ 7.99 (1H, d, J=8.8 Hz), 7.84 (3H, b), 7.60 (2H, d, J=8.8 Hz), 6.95 (1H, d, J=8.8 Hz), 6.84 (2H, d J=8.8 Hz), 4.68 (2H, t, J=4.8 Hz), 3.94 (2H, t, J=4.8 Hz), 3.74 (6H, m), 3.63 (2H, m), 3.01 (6H, s). HRMS (EI) m/z calcd. for [C₂₃H₂₈N₂O₄]⁺ 396.2049, found 396.2041.

### 2-(2-(2-(6-(4-(dimethylamino)phenyl)quinolin-2-yloxy)ethoxy)ethoxy)ethyl 4-methylbenzenesulfonate (32c)

Triethylamine (36 mg, 0.36 mmol) was added to a solution of compound 32b (36 mg, 0.09 mmol) in dichloromethane (5 ml), followed by the addition of DMAP (22 mg, 0.18 mmol). The solution was cooled to 0° C. and 4-methylbenzenesulfonic anhydride (59 mg, 0.18 mmol) was added in one portion. The reaction mixture was then warmed up to room temperature and stirred for 3 h. Water was added organic layer was separated, washed with brine, dried over sodium hydride and evaporated. The residue was purified by PTLC (3% methanol in dichloromethane as the developing solvent) to obtain product 32c (41.7 mg, Y: 83.4%). ¹H NMR (CDCl₃): δ 7.99 (1H, d, J=8.8 Hz), 7.84 (3H, b), 7.79 (2H, d, J=8.2 Hz), 7.60 (2H, d, J=8.8 Hz), 7.31 (2H, d, J=8.2 Hz), 6.92 (1H, d, J=8.8 Hz), 6.84 (2H, d J=8.8 Hz), 4.64 (2H, t, J=4.8 Hz), 4.16 (2H, t, J=4.8 Hz), 3.88 (2H, t, J=4.8 Hz), 3.66 (6H, m), 3.02 (6H, s), 2.41 (3H, s). HRMS (EI) m/z calcd. for [C₃₀H₃₄FN₂O₆S]⁺ 550.2138, found 550.2142.

### 4-(2-(2-(2-(2-fluoroethoxy)ethoxy)ethoxy)quinolin-6-yl)benzenamine (33a)

Compound 31a (51.7 mg, 0.14 mmol), 4-aminophenylboronic acid pinacolate (38 mg, 0.17 mmol), Palladium tetrakistriphenylphosphin (8.3 mg, 0.0072 mmol), tetra-butylammonium bromide (7 mg, 0.022 mmol) and sodium carbonate (2M aq. 0.36 ml, 0.72 mmol) was added to a mixed solvent of toluene (8 ml) and ethanol (2 ml). The reaction mixture was degassed by bubbling nitrogen for 10 min and then heated to 90° C. overnight. After cooled down to room temperature, the mixture was partitioned between ethyl acetate and water. The organic layer was separated, washed with brine, dried over anhydrous sodium sulfate and evaporated. The residue was purified by PTLC (using 40% ethyl acetate in hexane as the developing solvent) to obtain product 33a (40 mg, Y: 75%). ¹H NMR (CDCl₃): δ 7.99 (1H, d, J=8.8 Hz), 7.83 (3H, b), 7.50 (2H, d, J=8.8 Hz), 6.94 (1H, d, J=8.8 Hz), 6.78 (2H, d J=8.8 Hz), 4.68 (3H, m), 4.44 (1H, m), 3.94 (2H, t, J=4.8 Hz), 3.79 (8H, m).). HRMS (EI) m/z calcd. for [C₂₁H₂₃FN₂O₃]⁺ 370.1693, found 370.1688.

### 2-(2-(2-(6-(4-aminophenyl)quinolin-2-yloxy)ethoxy)ethoxy)ethanol (33b)

Compound 31b (100 mg, 0.28 mmol), 4-aminophenylboronic acid pinacolate (74 mg, 0.34 mmol), Palladium tetrakistriphenylphosphin (16 mg, 0.014 mmol) and sodium carbonate (2M aq. 0.7 ml, 1.4 mmol), tetra-butylammonium bromide (13.5 mg, 0.042 mmol) was added to a mixed solvent of toluene (8 ml) and ethanol (2 ml). The reaction mixture was degassed by bubbling nitrogen for 10 min and then heated to 90° C. overnight. After cooled down to room temperature, the mixture was partitioned between ethyl acetate and water. The organic layer was separated, washed with brine, dried over anhydrous sodium sulfate and evaporated. The residue was purified by PTLC (4% methanol in dichloromethane as the developing solvent) to obtain product 33b (92 mg, Y: 89%). ¹H NMR (CDCl₃): δ 7.99 (1H, d, J=8.8 Hz), 7.82 (3H, b), 7.50 (2H, d, J=8.6 Hz), 6.95 (1H, d, J=8.8 Hz), 6.78 (2H, d J=8.6 Hz), 4.68 (2H, t, J=4.8 Hz), 3.93 (2H, t, J=4.8 Hz), 3.74 (6H, m), 3.63 (2H, m). HRMS (EI) m/z calcd. for [C₂₁H₂₄N₂O₄]⁺ 368.1736, found 368.1741

### 4-(2-(2-(2-(2-fluoroethoxy)ethoxy)ethoxy)quinolin-6-yl)-N-methylbenzenamine (34a)

Sodium methoxide (0.5 M in methanol, 1.08 ml, 0.54 mmol) was added to a solution of compound 33a (40 mg, 0.108 mmol) in methanol (5 ml), followed by the addition of para-formaldehyde (16.2 mg, 0.54 mmol). The reaction mixture was refluxed for 1.5 h and cooled to 0° C. Sodium borohydride (24.5 mg, 0.65 mmol) was added in caution and reaction mixture was refluxed again for 1.5 h. The reaction mixture was cooled down to room temperature and dichloromethane was added. Organic layer was separated, washed with brine, dried over anhydrous sodium sulfate and evaporated. The residue was purified by PTLC (40% ethyl acetate in hexane as the eluant) to obtain product 34a (30 mg, Y: 72%). ¹H NMR (CDCl₃): δ 7.99 (1H, d, J=8.8 Hz), 7.83 (3H, br), 7.55 (2H, d, J=8.6 Hz), 6.94 (1H, d, J=8.8 Hz), 6.72 (2H, d J=8.6 Hz), 4.68 (3H, m), 4.44 (1H, m), 3.93 (2H, t, J=4.8 Hz), 3.75 (7H, m), 2.90 (3H, s). HRMS (EI) m/z calcd. for [C₂₂H₂₅FN₂O₃]⁺ 384.1849, found 384.1851.

### 2-(2-(2-(6-(4-(methylamino)phenyl)quinolin-2-yloxy)ethoxy)ethoxy)ethanol (34b)

Sodium methoxide (0.5 M in methanol, 2.02 ml, 1.1 mmol) was added to a solution of compound 33b (80 mg, 0.217 mmol) in methanol (5 ml), followed by the addition of para-formaldehyde (32.6 mg, 1.1 mmol). The reaction mixture was refluxed for 1.5 h and cooled to 0° C. Sodium borohydride (49.3 mg, 1.3 mmol) was added in caution and reaction mixture was refluxed again for 1.5 h. The reaction mixture was cooled down to room temperature and dichloromethane was added. Organic layer was separated, washed with brine, dried over anhydrous sodium sulfate and evaporated. The residue was purified by PTLC (30% ethyl acetate in hexane as the developing solvent) to obtain product 34b (76 mg, Y: 91.5%). ¹H NMR (CDCl₃): δ 7.99 (1H, d, J=8.8 Hz), 7.83 (3H, br), 7.54 (2H, d, J=8.4 Hz), 6.94 (1H, d, J=8.8 Hz), 6.72 (2H, d J=8.4 Hz), 4.68 (2H, t, J=4.8 Hz), 3.93 (2H, t, J=4.8 Hz), 3.73 (7H, m), 3.62 (2H, m), 2.90 (3H, s), 2.40 (1H, br). HRMS (EI) m/z calcd. for [C₂₂H₂₆N₂O₄]+ 382.1893, found 382.1894.

### tert-butyl 4-(2-(2-(2-(2-hydroxyethoxy)ethoxy)ethoxy)quinolin-6-yl)phenyl(methyl)carbamate (35b)

TBSCl (45 mg, 0.3 mmol) was added to a solution of 34b (76 mg, 0.2 mmol) in dichlormethane (8 ml) followed by the addition of imidazole (30 mg, 0.44 mmol). The reaction mixture was stirred at room temperature for 5 h. Water was added and organic layer was separated, washed with brine, dried over anhydrous sodium sulfate and evaporated. The residue was purified by PTLC (2% methanol in dichlormethane) to obtain solid 61 mg (Y: 62%). A portion of this material (56 mg, 0.113 mmol) was dissolved in anhydrous THF (5 ml). Di-tert-butyl dicarbonate (49 mg, 0.226 mmol) was added and the reaction mixture was refluxed overnight. Solvent was removed by vacuum and residue was purified by PTLC (30% ethyl acetate in hexane as the developing solvent) to obtain solid 60 mg (Y: 89%). Total of this solid (60 mg, 0.1 mmol) was dissolved in THF (5 ml). Tetrabutyl ammonium fluoride (1M in THF, 0.5 ml) was added and the reaction mixture was stirred at room temperature for 3 h. Solvent was removed and residue was purified by PTLC (3% methanol in dichloromethane as the developing solvent) to obtain product 35b (43 mg, Y: 51% from 34b). ¹H NMR (CDCl₃): δ 8.02 (1H, d, J=8.8 Hz), 7.87 (3H, b), 7.64 (2H, d, J=8.4 Hz), 7.34 (2H, d, J=8.4 Hz), 6.98 (1H, d, J=8.8 Hz), 4.69 (2H, t, J=4.8 Hz), 3.94 (2H, t, J=4.8 Hz), 3.74 (6H, m), 3.63 (2H, m), 3.32 (3H, s), 2.42 (1H, br), 1.49 (9H, s). HRMS (EI) m/z calcd. for [C₂₇H₃₄N₂O₆]⁺ 482.2417, found 482.2411.

### 2-(2-(2-(6-(4-(tert-butoxycarbonylmethyl)amino)phenyl)quinolin-2-yloxy)ethoxy)ethoxy)ethyl 4-methylbenzenesulfonate (35c)

Toluenesulfonyl chloride (33.8 mg, 0.172 mmol) was added to a solution of compound 35b (40 mg, 0.086 mmol) in pyridine (5 ml) at 0° C. The reaction mixture was warmed up to room temperature and stirred overnight. Solvent was removed by vacuum and the residue was partition between dichloromethane and water. Organic layer was separated, washed with brine, dried over sodium sulfate and evaporated. The residue was purified by PTLC (30% ethyl acetate in hexane as the developing solvent). Starting material 35b (9 mg) was also recovered, and product 35c (18 mg, Y: 43% base on the amount of 35b consumed) was obtained. ¹H NMR (CDCl₃): δ 8.02 (1H, d, J=8.8 Hz), 7.88 (3H, b), 7.79 (2H, d, J=8.2 Hz), 7.64 (2H, d, J=8.4 Hz), 7.32 (4H, m), 6.96 (1H, d, J=8.8 Hz), 4.65 (2H, t, J=4.8 Hz), 4.16 (2H, t, J=4.8 Hz), 3.89 (2H, t, J=4.8 Hz), 3.67 (6H, m), 3.32 (3H, s), 2.42 (3H, s), 1.49 (9H, s). HRMS (EI) m/z calcd. for [C₃₄H₄₀N₂O₈S]⁺ 636.2505, found 636.2487.

### F-18 Labeling

[¹⁸F]/F⁻ on Sep-Pak Light QMA cartridge was provided by the Cyclotron at University of Pennsylvania. The [¹⁸F]/F⁻ was eluted from QMA cartridge with 1.1 mL solution of acetonitrile (1 mL) and water (0.1 mL) containing Kryptofix (11 mg) and potassium carbonate (2.6 mg). The water was azeotropically evaporated from this mixture using HPLC grade acetonitrile (2×1.0 mL) in a heating block at 120° C. under a nitrogen flow. After final drying sequence, 1 mg of precursor 14c, 20c, 31c or 35c in DMSO (0.3 mL) was added to the ¹⁸F residue. The mixture was irradiated with microwave (Resonance Instruments Model 521) at 50 Watts, maximum 100° C. for 1 min. For 14c, 20c or 35c, 10% HCl (0.5 mL) was added and reaction mixture was irradiated again with microwave at 50 W at maximum 100° C. for 1 min to remove the Boc protection group and then neutralized with 2N NaOH. This deprotection step was not needed for 31c. Solid-phase extraction was performed by using a C4 3 cc cartridge (Grace VyDac), pre-washed with ethanol (10 mL) and water (10 mL). Reaction mixture was diluted with water (4 mL) and then loaded onto the C4 cartridge. The cartridge was washed with water (2 mL) and the labeled compound was eluted with CH₃CN (0.5 mL). Eluted compound was purified by semi-preparative HPLC and the quality control of these F-18 labeled compounds [¹⁸F]13a, [¹⁸F]19a, [¹⁸F]31a and [¹⁸F]34a were performed by analytical HPLC coeluting with nonradioactive standard 13a, 19a, 31a and 34a. The area of UV peak corresponding to the product was compared with a standard calibration curve and was used to determine the SA of [¹⁸F] 13a, [¹⁸F]19a, [¹⁸F]31a and [¹⁸F]34a. The SA of [¹⁸F] 13a, [¹⁸F]19a, [¹⁸F]31a and [¹⁸F]34a were estimated at about 500-2000 Ci/mmol. The complete synthesis required about 50-70 min; the radiochemical purity was >99% and the radiochemical yield was about 30% (decay corrected) for all these compounds.

Semiprep HPLC condition: Agilent 1100 series HPLC, column: Phenonemex Gemini C18 semi-prep column (10×250 mm, 5 μm); solvent system: acetonitrile/water 7/3 at flow rate 4 mL/min with UV at 350 nm for [¹⁸F]13a and [¹4]19a, UV at 305 nm for [¹⁸F]31a and [¹⁸F]34a. Analytical HPLC condition: Agilent 1100 series HPLC, column: Phenomenex Gemini C18 analytical column (4.6×250 mm, 5 μm); solvent system: acetonitrile /ammonium formate buffer (10 mM) 8/2; flow rate 1 mL/min with UV at 350 nm; retention time of [¹⁸F]13a and [¹⁸F]19a were 5.6 and 5.7 min, respectively. [¹⁸F]31a and [¹⁸F]34a were detected with UV at 305 nm, retention time of [¹⁸F]31a at solvent system: acetonitrile /ammonium formate buffer (10 mM) 8/2; flow rate 1 mL/min was 7.9 min and the retention time of [¹⁸F]34a at solvent system: acetonitrile /ammonium formate buffer (10 mM) 7/3; flow rate 1 mL/min was 7.8 min.

TABLE I
Ki values of Phen-Nap, Nap-Phen derivatives

| structure | R₁ | R₂ | 1. compound | Ki (nM) |
|---|---|---|---|---|
| 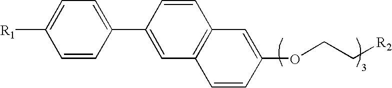 | —NH₂ —N(CH₃)₂ —NHCH₃ —OH | —F —OH —F —F —OH —F | 11a 11b 12a 13a 13b 26 | 12.5 ± 1.2 22 ± 4.5 3.1 ± 0.5 1.6 ± 0.4 1.0 ± 0.2 16.5 ± 2.5 |
| 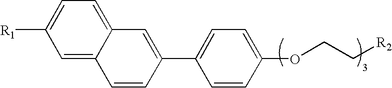 | —NH₂ —N(CH₃)₂ —NHCH₃ —OH | —F —OH —F —OH —F —OH —F | 17a 17b 18a 18b 19a 19b 28 | 20 ± 5.0 27 ± 3.0 5.6 ± 1.8 6.5 ± 1.8 3.0 ± 0.8 6.0 ± 0.5 16.0 ± 4.5 |
| 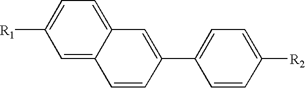 | —NH₂ —N(CH₃)₂ OH | —OH —OCH₃ —OH —OCH₃ | 16 23 24 25 | 33.5 ± 11 1.4 ± 0.4 4.2 ± 0.4 1.5 ± 1.4 |
| 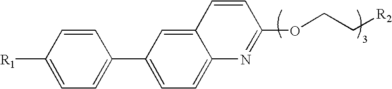 | —NH₂ —N(CH₃)₂ —NHCH₃ | —F —OH —F OH —F —OH | 33a 33b 32a 32b 34a 34b | 102 ± 32 109 ± 11.2 4.4 ± 0.6 4.7 ± 0.9 5.5 ± 0.5 5.0 ± 1.0 |

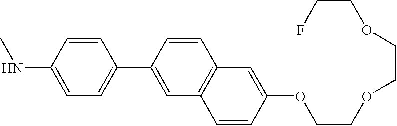

### [4-(6-{2-[2-(2-Fluoro-ethoxy)-ethoxy]-ethoxy}-naphthalen-2-yl)-phenyl]-methyl-amine

### Biodistribution in ICR Mice After an iv Injection of [¹⁸F][4-(6-{2-[2-(2-Fluoro-ethoxy)-ethoxy]-ethoxy}-naphthalen-2-yl)-phenyl]-methyl-amine in 1% EtOH/0.1% BSA in Water

(% dose/organ, avg of 3 mice±SD)

| Organ | 2 min | 30 min | 1 hr | 2 hr |
|---|---|---|---|---|
| Blood | 3.50 ± 0.18 | 2.99 ± 0.48 | 2.73 ± 0.41 | 3.59 ± 0.53 |
| Heart | 0.63 ± 0.08 | 0.23 ± 0.02 | 0.18 ± 0.01 | 0.19 ± 0.03 |
| Muscle | 11.8 ± 9.69 | 12.7 ± 0.64 | 10.3 ± 0.54 | 9.00 ± 0.56 |
| Lung | 1.08 ± 0.05 | 0.53 ± 0.10 | 0.41 ± 0.05 | 0.48 ± 0.16 |
| Kidney | 3.27 ± 0.60 | 1.45 ± 0.12 | 1.05 ± 0.25 | 0.88 ± 0.10 |
| Spleen | 0.47 ± 0.03 | 0.23 ± 0.01 | 0.23 ± 0.02 | 0.19 ± 0.03 |
| Liver | 26.5 ± 2.18 | 17.1 ± 1.54 | 16.3 ± 1.39 | 13.5 ± 1.77 |
| Skin | 3.66 ± 0.74 | 7.08 ± 0.26 | 6.53 ± 0.12 | 4.60 ± 0.29 |
| Brain | 3.43 ± 0.51 | 0.95 ± 0.08 | 0.75 ± 0.06 | 0.88 ± 0.06 |
| Bone | 7.38 ± 3.40 | 3.36 ± 0.09 | 4.61 ± 0.36 | 7.07 ± 0.57 |

(% dose/g, avg of 3 mice±SD)

| Organ | 2 min | 30 min | 1 hr | 2 hr |
|---|---|---|---|---|
| Blood | 2.08 ± 0.20 | 1.72 ± 0.29 | 1.41 ± 0.33 | 2.22 ± 0.37 |
| Heart | 5.44 ± 0.25 | 2.04 ± 0.30 | 1.46 ± 0.24 | 1.79 ± 0.19 |
| Muscle | 1.21 ± 0.98 | 1.27 ± 0.06 | 0.92 ± 0.09 | 0.97 ± 0.06 |
| Lung | 5.73 ± 0.19 | 2.78 ± 0.30 | 1.99 ± 0.26 | 2.44 ± 0.37 |
| Kidney | 8.88 ± 1.80 | 3.98 ± 0.64 | 2.75 ± 0.35 | 2.52 ± 0.18 |
| Spleen | 5.11 ± 0.49 | 2.12 ± 0.33 | 1.81 ± 0.17 | 2.08 ± 0.44 |
| Liver | 19.7 ± 3.24 | 13.4 ± 1.30 | 9.46 ± 1.50 | 11.9 ± 1.87 |
| Skin | 1.01 ± 0.16 | 1.90 ± 0.08 | 1.56 ± 0.18 | 1.32 ± 0.09 |
| Brain | 7.26 ± 1.28 | 2.15 ± 0.25 | 1.58 ± 0.18 | 1.91 ± 0.12 |
| Bone | 2.14 ± 0.82 | 0.97 ± 0.03 | 1.19 ± 0.21 | 2.18 ± 0.19 |

Log P of [4-(6-{2-[2-(2-Fluoro-ethoxy)-ethoxy]-ethoxy}-naphthalen-2-yl)-phenyl]-methyl-amine: 3.22 (octanol/0.05 M Na₂HPO₄—buffer (pH 7.4))

### Biodistribution in ICR Mice After an iv Injection of [¹⁸F]19a in 3% EtOH/0.1% BSA in Water

(% dose/organ, avg of 3 mice±SD)

| Organ | 2 min | 30 min | 1 hr | 2 hr |
|---|---|---|---|---|
| Blood | 11.1 ± 0.91 | 6.94 ± 0.59 | 4.94 ± 0.09 | 5.19 ± 0.14 |
| Heart | 0.63 ± 0.02 | 0.21 ± 0.02 | 0.18 ± 0.01 | 0.16 ± 0.01 |
| Muscle | 7.42 ± 1.87 | 7.22 ± 0.42 | 7.86 ± 0.68 | 6.02 ± 0.62 |
| Lung | 1.76 ± 0.20 | 0.67 ± 0.05 | 0.41 ± 0.07 | 0.45 ± 0.11 |
| Kidney | 3.06 ± 0.21 | 1.38 ± 0.26 | 1.37 ± 0.25 | 0.88 ± 0.12 |
| Spleen | 0.79 ± 0.29 | 0.53 ± 0.04 | 0.30 ± 0.00 | 0.31 ± 0.02 |
| Liver | 32.0 ± 1.81 | 25.7 ± 0.95 | 15.7 ± 1.34 | 10.6 ± 0.65 |
| Skin | 2.51 ± 0.07 | 4.93 ± 0.08 | 4.46 ± 0.05 | 2.81 ± 0.29 |
| Brain | 2.51 ± 0.29 | 0.59 ± 0.07 | 0.48 ± 0.06 | 0.42 ± 0.06 |
| Bone | 4.42 ± 0.82 | 2.59 ± 0.17 | 2.60 ± 0.41 | 4.67 ± 0.26 |

(% dose/g, avg of 3 mice±SD)

| Organ | 2 min | 30 min | 1 hr | 2 hr |
|---|---|---|---|---|
| Blood | 6.66 ± 0.86 | 4.15 ± 0.59 | 3.14 ± 0.37 | 3.05 ± 0.26 |
| Heart | 5.67 ± 0.42 | 1.74 ± 0.19 | 1.69 ± 0.35 | 1.40 ± 0.08 |
| Muscle | 0.77 ± 0.14 | 0.75 ± 0.09 | 0.87 ± 0.03 | 0.62 ± 0.11 |
| Lung | 9.19 ± 1.18 | 3.44 ± 0.49 | 2.68 ± 0.48 | 2.48 ± 0.48 |
| Kidney | 7.88 ± 0.94 | 3.61 ± 0.98 | 3.82 ± 1.54 | 2.41 ± 0.42 |
| Spleen | 6.94 ± 0.99 | 5.03 ± 1.18 | 3.60 ± 0.15 | 3.38 ± 0.52 |
| Liver | 27.0 ± 0.91 | 21.5 ± 2.08 | 14.1 ± 1.85 | 8.85 ± 0.76 |
| Skin | 0.70 ± 0.04 | 1.37 ± 0.11 | 1.32 ± 0.18 | 0.77 ± 0.13 |
| Brain | 5.55 ± 0.77 | 1.29 ± 0.19 | 1.05 ± 0.20 | 0.92 ± 0.10 |
| Bone | 1.32 ± 0.18 | 0.78 ± 0.10 | 0.82 ± 0.21 | 1.37 ± 0.15 |

Log P of [¹⁸F]19a: 2.60 (octanol/0.05 M Na₂HPO₄—buffer (pH 7.4))

### Biodistribution in ICR Mice After an iv Injection of [¹⁸F]34a in 5% EtOH/0.1% BSA in Water

(% dose/organ, avg of 3 (*or 2) mice±SD)

| Organ | 2 min* | 30 min | 1 hr | 2 hr |
|---|---|---|---|---|
| Blood | 4.29 ± 0.20 | 2.51 ± 0.18 | 2.87 ± 0.20 | 2.58 ± 0.26 |
| Heart | 1.26 ± 0.20 | 0.36 ± 0.05 | 0.29 ± 0.03 | 0.19 ± 0.01 |
| Muscle | 6.40 ± 0.14 | 14.5 ± 2.59 | 13.5 ± 0.83 | 9.55 ± 0.31 |
| Lung | 1.34 ± 0.03 | 0.54 ± 0.06 | 0.48 ± 0.06 | 0.36 ± 0.05 |
| Kidney | 5.22 ± 0.33 | 1.69 ± 0.22 | 1.36 ± 0.09 | 1.00 ± 0.24 |
| Spleen | 0.47 ± 0.08 | 0.21 ± 0.00 | 0.17 ± 0.01 | 0.14 ± 0.02 |
| Liver | 21.2 ± 0.76 | 14.2 ± 0.91 | 11.8 ± 0.65 | 8.29 ± 1.09 |
| Skin | 2.13 ± 0.27 | 3.70 ± 0.28 | 5.67 ± 0.85 | 4.12 ± 0.12 |
| Brain | 4.61 ± 0.60 | 1.02 ± 0.11 | 0.90 ± 0.08 | 0.85 ± 0.12 |
| Bone | 3.82 ± 0.33 | 3.87 ± 0.43 | 5.31 ± 0.31 | 7.84 ± 0.91 |

(% dose/g, avg of 3 (*or 2) mice±SD)

| Organ | 2 min* | 30 min | 1 hr | 2 hr |
|---|---|---|---|---|
| Blood | 2.50 ± 0.14 | 1.49 ± 0.15 | 1.61 ± 0.12 | 1.52 ± 0.23 |
| Heart | 10.7 ± 2.06 | 2.86 ± 0.30 | 2.20 ± 0.31 | 1.71 ± 0.13 |
| Muscle | 0.65 ± 0.01 | 1.50 ± 0.21 | 1.33 ± 0.17 | 0.98 ± 0.08 |
| Lung | 6.83 ± 0.07 | 2.87 ± 0.32 | 2.42 ± 0.34 | 1.96 ± 0.29 |
| Kidney | 13.6 ± 0.10 | 4.41 ± 0.49 | 3.57 ± 0.64 | 2.47 ± 0.45 |
| Spleen | 4.11 ± 1.02 | 2.16 ± 0.05 | 1.89 ± 0.27 | 1.48 ± 0.20 |
| Liver | 18.2 ± 0.95 | 11.9 ± 1.63 | 8.99 ± 1.19 | 6.72 ± 0.58 |
| Skin | 0.58 ± 0.07 | 1.03 ± 0.12 | 1.47 ± 0.12 | 1.12 ± 0.05 |
| Brain | 10.2 ± 1.09 | 2.23 ± 0.29 | 1.88 ± 0.20 | 1.90 ± 0.25 |
| Bone | 1.11 ± 0.11 | 1.16 ± 0.20 | 1.49 ± 0.18 | 2.33 ± 0.43 |

Log P of [¹⁸F]34a: 3.23 (octanol/0.05 M Na₂HPO₄—buffer (pH 7.4))

### Biodistribution in ICR Mice After an iv Injection of [¹⁸F]32a in 1% EtOH/0.1% BSA in Water

(% dose/organ, avg of 3 mice±SD)

| Organ | 2 min | 30 min | 1 hr | 2 hr |
|---|---|---|---|---|
| Blood | 3.32 ± 0.46 | 1.80 ± 0.14 | 2.40 ± 0.19 | 2.00 ± 0.25 |
| Heart | 1.36 ± 0.20 | 0.26 ± 0.06 | 0.25 ± 0.03 | 0.15 ± 0.01 |
| Muscle | 8.87 ± 2.47 | 12.3 ± 2.18 | 11.0 ± 0.64 | 8.16 ± 1.14 |
| Lung | 1.54 ± 0.16 | 0.42 ± 0.07 | 0.40 ± 0.01 | 0.27 ± 0.00 |
| Kidney | 5.25 ± 0.89 | 1.55 ± 0.33 | 1.24 ± 0.19 | 0.73 ± 0.02 |
| Spleen | 0.51 ± 0.09 | 0.15 ± 0.03 | 0.15 ± 0.04 | 0.09 ± 0.01 |
| Liver | 19.4 ± 3.67 | 11.2 ± 1.65 | 9.75 ± 1.79 | 6.34 ± 0.87 |
| Skin | 1.68 ± 0.22 | 3.86 ± 1.41 | 3.53 ± 0.16 | 3.11 ± 0.48 |
| Brain | 3.56 ± 0.47 | 1.07 ± 0.24 | 0.86 ± 0.14 | 0.66 ± 0.03 |
| Bone | 4.63 ± 0.32 | 2.92 ± 0.27 | 3.91 ± 0.37 | 5.19 ± 0.80 |

(% dose/g, avg of 3 mice±SD)

| Organ | 2 min | 30 min | 1 hr | 2 hr |
|---|---|---|---|---|
| Blood | 1.80 ± 0.28 | 0.99 ± 0.12 | 1.37 ± 0.14 | 1.13 ± 0.15 |
| Heart | 10.1 ± 1.14 | 1.99 ± 0.43 | 2.07 ± 0.23 | 1.28 ± 0.09 |
| Muscle | 0.83 ± 0.19 | 1.20 ± 0.28 | 1.09 ± 0.09 | 0.81 ± 0.14 |
| Lung | 7.63 ± 0.46 | 2.31 ± 0.42 | 2.21 ± 0.19 | 1.43 ± 0.03 |
| Kidney | 13.0 ± 3.04 | 3.61 ± 0.65 | 3.38 ± 0.38 | 1.78 ± 0.06 |
| Spleen | 4.95 ± 0.78 | 1.56 ± 0.28 | 1.64 ± 0.13 | 1.16 ± 0.07 |
| Liver | 16.3 ± 4.96 | 8.02 ± 0.65 | 8.10 ± 1.59 | 5.22 ± 0.49 |
| Skin | 0.42 ± 0.06 | 0.98 ± 0.33 | 0.94 ± 0.06 | 0.82 ± 0.15 |
| Brain | 7.76 ± 1.25 | 2.33 ± 0.68 | 1.92 ± 0.31 | 1.45 ± 0.11 |
| Bone | 1.25 ± 0.06 | 0.81 ± 0.11 | 1.11 ± 0.14 | 1.47 ± 0.27 |

Log P of [¹⁸F]32a: 2.77 (octanol/0.1 M Na₂HPO₄—buffer (pH 7.4)

It will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

What is claimed:

1. A compound of Formula I: 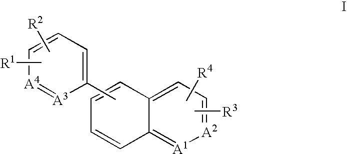
or a pharmaceutically acceptable salt or prodrug thereof, wherein:
A¹ and A² are independently C or N;
A³ and A⁴ are independently C or N;
R¹ and R⁴ are each independently:
NR′R″, wherein R′ and R″ are independently hydrogen, (C₁₋₄)alkyl, hydroxy(C₁₋₄alkyl or halo(C₁₋₄)alkyl; hydroxy; C₁₋₄ alkoxy; hydroxy(C₁₋₄alkyl; halogen; cyano; hydrogen; nitro; (C₁-C₄)alkyl; halo (C₁-C₄)alkyl; formyl; —O—CO(C₁₋₄ alkyl);
—COO(C₁₋₄ alkyl); —NHCO(C₁₋₄ alkyl); or radiohalogen;
R² and R³ are each independently hydrogen or fragment i, ii or iii, wherein:
i is: 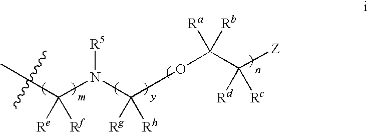
wherein, n is an integer from 1 to 10; m is an integer from 0 to 5; y is an integer from 0 to 5; R⁵ is hydrogen, (C₁₋₄)alkyl, or hydroxy(C₁₋₄)alkyl; R^{a}, R^{b}, R^{c}, R^{d}, R^{e}, R^{f}, R^{g} and R^{h} are each independently hydrogen, halogen, hydroxy, (C₁₋₄)alkoxy, C₁₋₄ alkyl, or hydroxy(C₁₋₄alkyl; and
Z is:
a) X, wherein X is hydrogen, hydroxy, halogen, radiohalogen, (C₁₋₄)alkoxy, hydroxy(C₁₋₄alkyl, halo(C₁₋₄)alkyl, radiohalo(C₁₋₄)alkyl or NR^{x}R^{y},
wherein R^{x} and R^{y} are each independently hydrogen, (C₁₋₄)alkyl, hydroxy(C₁₋₄alkyl, halo(C₁₋₄)alkyl or radiohalo(C₁₋₄)alkyl;
b) benzoyloxy, phenyl(C₁₋₄)alkyl, aryloxy or (C₆₋₁₀)aryl, each of which is substituted by X; or
c) Zc: 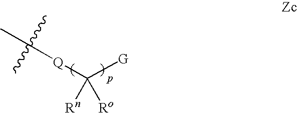
wherein, p is an integer from 1 to 4, Q is O or NR⁵; G is —C═C—(R^{G})X or —C≡C—X, wherein R^{G} is hydrogen or (C₁₋₄)alkyl, R″ and
R^{o} are independently hydrogen, hydroxyl or (C₁₋₄)alkyl;
ii is: 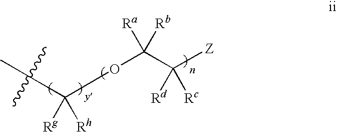
wherein, y′ is an integer from 0 to 5;
and iii is: 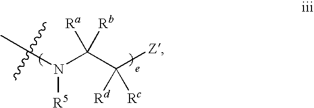
wherein, e is 0 or 1;
provided that,
a) X is F or ¹⁸F or contains F or ¹⁸F; or
b) one of R¹ and R⁴ is F, ¹⁸F, ¹²³I, ¹²⁵I, ¹³¹I, ⁷⁶Br, ⁷⁷Br or Br; or
c) one of R² and R³ is other than hydrogen.

2. The compound of claim 1 comprising at least one radiohalogen, wherein said radiohalogen is ¹²³I, ¹²⁵I, ¹³¹I, ¹⁸F, ¹⁹F, ⁷⁶Br or ⁷⁷Br.

3. The compound of claim 1 comprising at least one radiohalogen, wherein said radiohalogen is ¹⁸F or ¹²³I.

4. The compound of claim 1, wherein R² is hydrogen.

5. The compound of claim 1, wherein at least one of A¹ and A² is N.

6. The compound of claim 1, wherein A¹ is C and A² is N.

7. The compound of claim 1, wherein A¹ and A² are each C.

8. The compound of claim 1, wherein at least one of A³ and A⁴ is N.

9. The compound of claim 1, wherein A³ is C and A⁴ is N.

10. The compound of claim 1, wherein A³ and A⁴ are each C.

11. The compound of claim 1, wherein R³ is 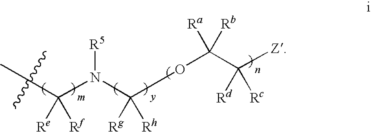
wherein,
n is an integer from 1 to 10;
m is an integer from 0 to 5;
y is an integer from 1 to 5; and
R⁵ is hydrogen, (C₁₋₄)alkyl, or hydroxy(C₁₋₄)alkyl.

12. The compound of claim 11, wherein:
n is an integer from 1 to 6;
m is an integer from 0 to 3; and
y is an integer from 1 to 3.

13. The compound of claim 11, wherein:
n is an integer from 2 to 6;
m is 0; and
y is 2.

14. The compound of claim 11, wherein R^{a}, R^{b}, R^{c}, R^{d}, R^{e}, R^{f}, R^{g} and R^{h} are each hydrogen.

15. The compound of claim 11, having the structure: 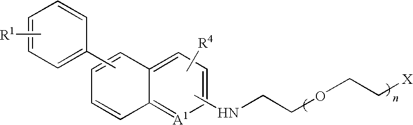
wherein,
A¹ is C or N;
n is an integer from 1 to 6;
R¹ is hydroxy, (C₁₋₄)alkoxy, —NHCO(C₁₋₄ alkyl) or NR′R″, wherein R′ and R″ are independently hydrogen or (C₁₋₄)alkyl;
R⁴ is hydrogen, (C₁₋₄)alkyl, (C₁₋₄)alkoxy, halogen or radiohalogen; and
X is hydrogen, halogen, radiohalogen, (C₁₋₄)alkoxy, hydroxy or NR^{a}R^{b};
provided that,
X is ¹⁸F, or R⁴ is ¹²³I, ¹²⁵I or ¹³¹I.

16. The compound of claim 15, wherein:
n is 3;
R¹ is hydroxy or —NR′R″, wherein R′ and R″ are independently hydrogen or C₁₋₄ alkyl;
R⁴ is hydrogen, halogen or radiohalogen; and
X is hydroxy, halogen or radiohalogen.

17. The compound of claim 1, wherein R³ is 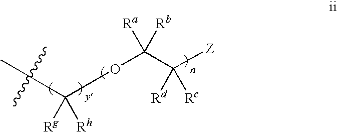
wherein,
n is an integer from 1 to 10;
y′ is an integer from 0 to 5;
provided that,
X is ¹⁸F, or R⁴ is ¹²³I, ¹²⁵I or ¹³¹I.

18. The compound of claim 1, having the following structure: 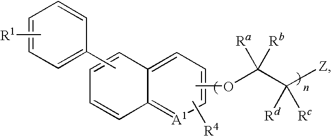
wherein,
A¹ is C or N;
n is an integer from 2 to 6;
R¹ is hydroxy, (C₁₋₄)alkoxy, —NHCO(C₁₋₄ alkyl) or NR′R″, wherein R′ and R″ are independently hydrogen or (C₁₋₄)alkyl;
R⁴ is hydrogen, (C₁₋₄)alkyl, (C₁₋₄)alkoxy, halogen or radiohalogen; and
X is hydroxy, halogen or radiohalogen;
provided that,
X is ¹⁸F, or R⁴ is ¹²³I, ¹²⁵I or ¹³¹I.

19. The compound of claim 18, wherein A¹ is N.

20. The compound of claim 18, wherein R^{a}, R^{b}, R^{c} and R^{d} are in each instance hydrogen.

21. The compound of claim 18, wherein n is 3.

22. The compound of claim 1, wherein R³ is 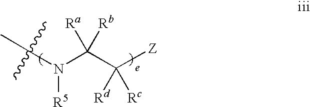
wherein,
e is 0 or 1;
provided that,
X contains ¹⁸F, or R⁴ is ¹²³I, ¹²⁵I or ¹³¹I.

23. The compound of claim 22, wherein e is 1.

24. The compound of 23, wherein:
Z is:
X, wherein X is hydrogen, halogen, radiohalogen, (C₁₋₄)alkoxy, hydroxy or NR′R″; or
Zc: 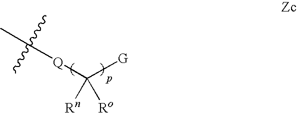
wherein, p is an integer from 1 to 4, Q is O or NR⁵; G is —C═C—(R^{G})X or —C≡C—X, wherein R^{G} is hydrogen or (C₁₋₄)alkyl, and Rⁿ and R^{o} are independently hydrogen, hydroxy or (C₁₋₄)alky.

25. The compound of claim 1 that is: 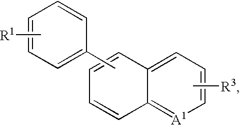
wherein A¹ is C or N; 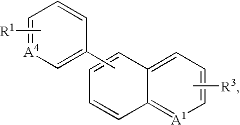
wherein, R¹ is hydroxy or NR′R″; A¹ is C or N and A⁴ is N; 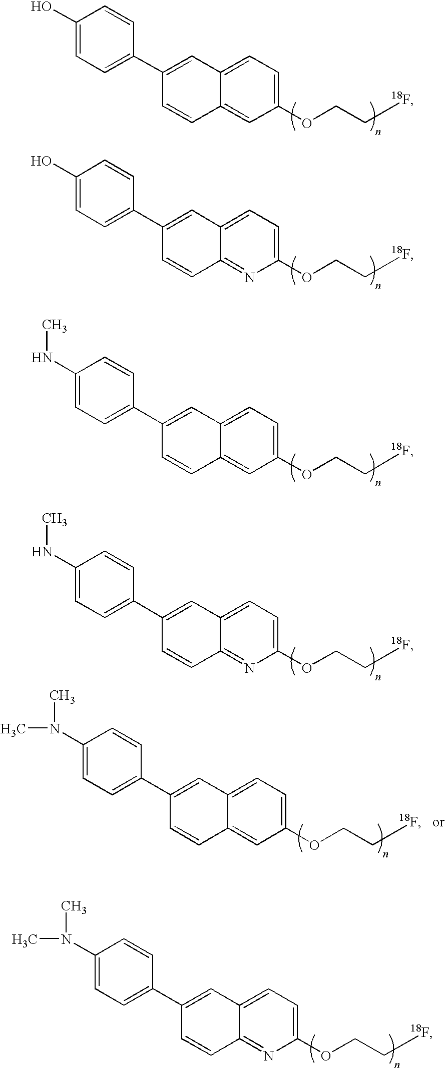
wherein n is an integer from 1 to 10; 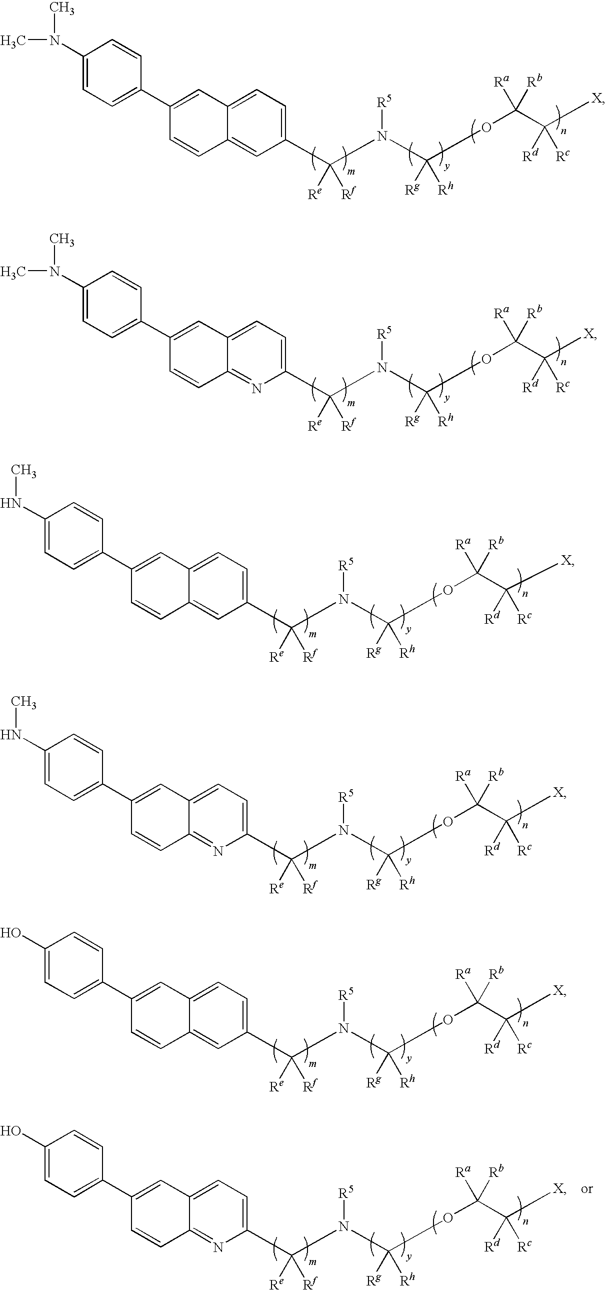 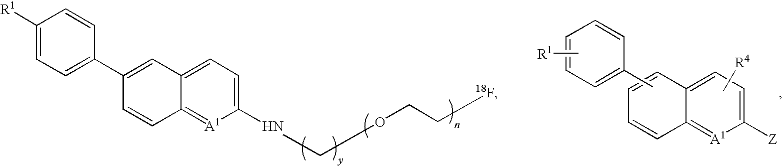
wherein, R¹ is hydroxy or NR′R″, wherein R′ and R″ are independently hydrogen or (C₁₋₄)alkyl, A¹ is C or N, Z is X, wherein X is hydrogen, hydroxy or (C₁₋₄)alkoxy and R⁴ is I, ¹²³I, ¹²⁵I, ¹³¹I, Br, ⁷⁶Br or ⁷⁷Br; 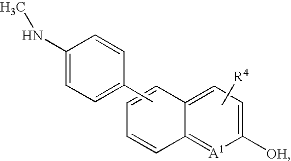
wherein A¹ is C or N, and R⁴ is I, ¹²³I, ¹²⁵I, ¹³¹I, Br, ⁷⁶Br or ⁷⁷Br; 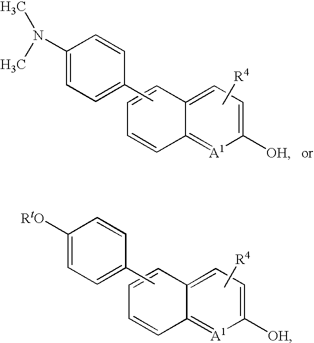
wherein A¹ is C or N, and R⁴ is I, ¹²³I, ¹²⁵I, ¹³¹I, Br, ⁷⁶Br or ⁷⁷Br; and R^{t} is (C₁₋₄)alkyl; 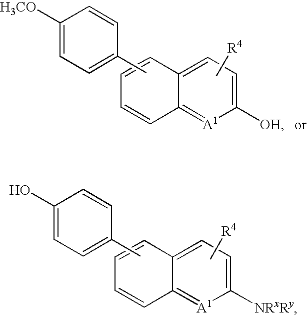
wherein R^{x} and R^{y} are each independently hydrogen or (C₁₋₄)alkyl, A¹ is C or N, and R⁴ is F, ¹⁸F, I, ¹²³I, ¹²⁵I, ¹³¹I, Br, ⁷⁶Br or ⁷⁷Br; 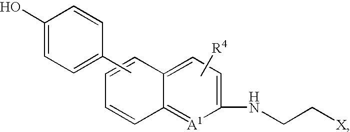
wherein A¹ is C or N, R⁴ is F, ¹⁸F, I, ¹²³I, ¹²⁵I, ¹³¹I, Br, ⁷⁶Br or ⁷⁷Br, and X is hydroxy, F or ¹⁸F; 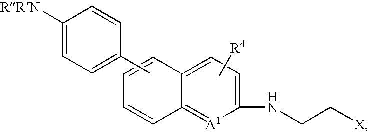
wherein R′ and R″ are each independently hydrogen or C₁₋₄ (C₁₋₄)alkyl, A¹ is C or N, R⁴ is F, ¹⁸F, I, ¹²³I, ¹²⁵I, ¹³¹I, Br, ⁷⁶Br or ⁷⁷Br, and X is hydroxy, F or ¹⁸F; 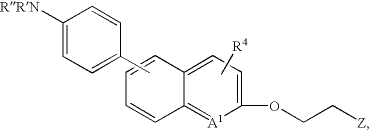
wherein R′ and R″ are each independently hydrogen or (C₁₋₄)alkyl, A¹ is C or N, R⁴ is F, ¹⁸F, I, ¹²³I, ¹²⁵I, ¹³¹I, Br, ⁷⁶Br, and Z is X, wherein X is hydroxy, F or ¹⁸F; 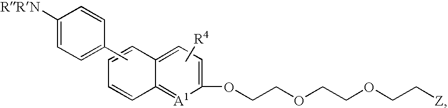
wherein R′ and R″ are each independently hydrogen or (C₁₋₄)alkyl, A¹ is C or N, R⁴ is F, ¹⁸F, I, ¹²³I, ¹²⁵I, ¹³¹I Br, ⁷⁶Br or ⁷⁷Br, and Z is X, wherein X is hydroxyl, F, ¹⁸F or Zc, wherein Zc is: 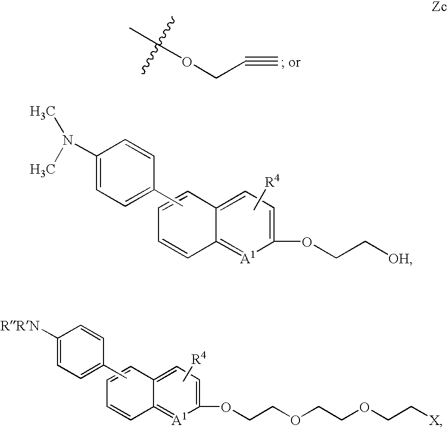
wherein, in compounds 32 and 33, if present, R⁴ is a radiohalogen, one of R′ and R″ is (C₁₋₄)alkyl, the other is hydrogen or (C₁₋₄)alkyl, A¹ is C or N, and X is F or ¹⁸F; 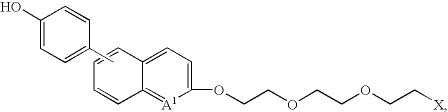
wherein, A¹ is C or N, and X is F or ¹⁸F; 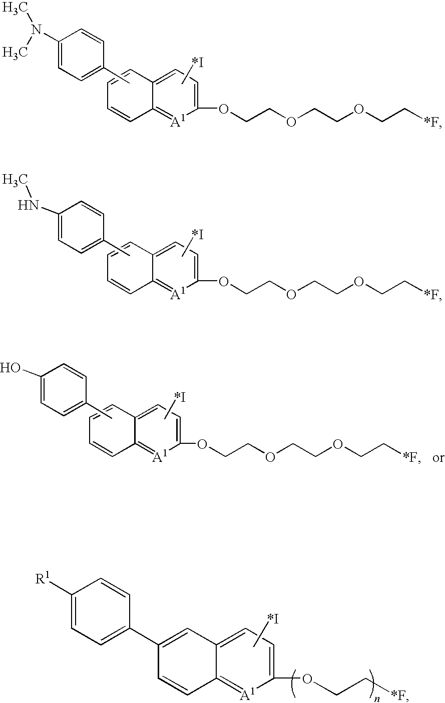
wherein R¹ is hydroxy or NR′R″, wherein R′ and R″ are independently hydrogen or C₁₋₄ alkyl, A¹ is C or N, n is 2, 3 or 4; and I and *F are non-radiolabeled or radiolabeled; 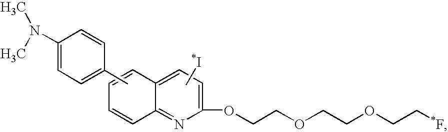
wherein *I and *F are non-radiolabeled or radiolabeled; or 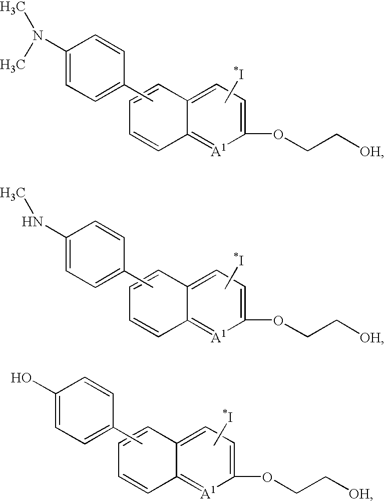
wherein, A¹ is C or N, and *I is radiolabeled or non-radiolabeled; 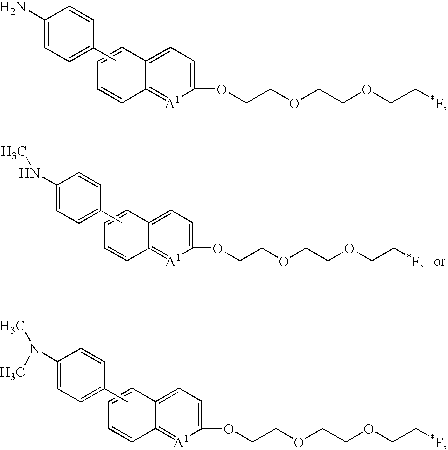
wherein, *F is radiolabeled or non-radiolabeled fluorine; 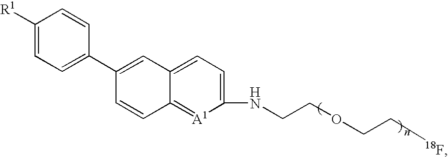
wherein, A¹ is C or N, R¹ is —N(Me)₂, —NHMe or hydroxy, and n is 1, 2 or 3;

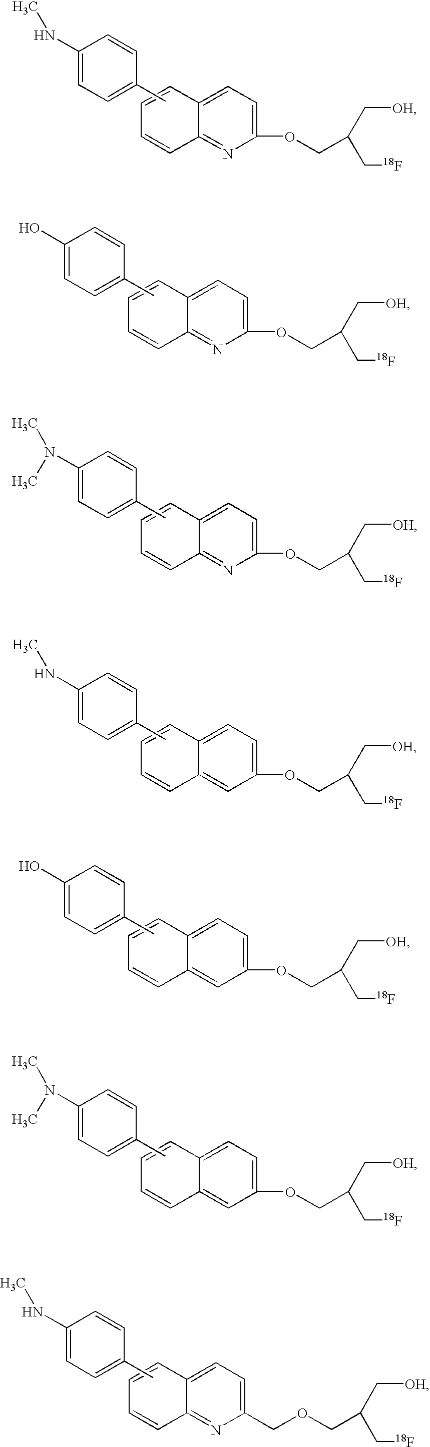

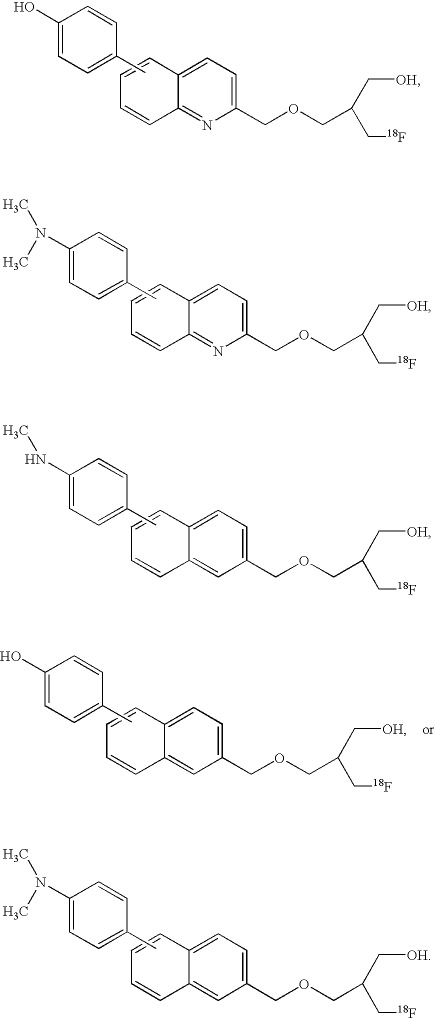

26. A compound of Formula II: 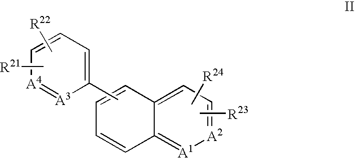
or a pharmaceutically acceptable salt or prodrug thereof, wherein:
A¹ and A² are independently C or N;
A³ and A⁴ are independently C or N;
R²¹ and R²⁴ are each independently:
NR′R″, wherein R′ and R″ are independently hydrogen, (C₁₋₄)alkyl, hydroxy(C₁₋₄alkyl or halo(C₁₋₄)alkyl; hydroxy; C₁₋₄ alkoxy; hydroxy(C₁₋₄alkyl;
halogen; cyano; hydrogen; nitro; (C₁-C₄)alkyl; halo(C₁-C₄)alkyl; formyl; —NHCO(C₁₋₄) alkyl; or radiohalogen;
R²² and R²³ are each independently hydrogen or fragment i, ii, iii or iv, wherein:
fragment i is: 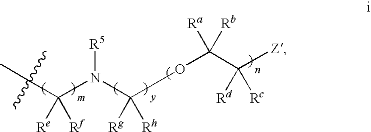
wherein, n is an integer from 1 to 10; m is an integer from 0 to 5; y is an integer from 1 to 5; R⁵ is hydrogen, (C₁₋₄)alkyl, or hydroxy(C₁₋₄alkyl; R^{a}, R^{b}, R^{c}, R^{d}, R^{e}, R^{f}, R^{g} and R^{h} are each independently hydrogen, halogen, hydroxy, (C₁₋₄)alkoxy, (C₁₋₄)alkyl or hydroxy(C₁₋₄alkyl; and
Z′ is:
a) -Ch, wherein -Ch is described fully below;
b) one of the following groups, each of which contains a -Ch directly bound to the aromatic ring: benzoyloxy, phenyl(C₁₋₄)alkyl, aryloxy and C₆₋₁₀ aryl; or
c) Z′c: 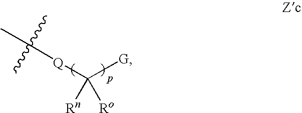
wherein, p is an integer from 1 to 4, Q is O or NR⁵; G is —C═C—(R^{G})Ch or —C≡C-Ch, wherein R^{G} is hydrogen or (C₁₋₄)alkyl; Rⁿ and R^{o} are independently hydrogen, hydroxy or (C₁₋₄)alkyl, and R⁵ and -Ch are as described below;
fragment ii is: 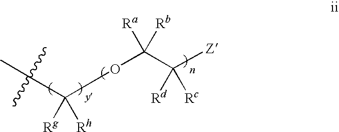
wherein, y′ is an integer from 0 to 5, and n, R^{a}, R^{b}, R^{c}, R^{d}, and Z′ are as described above;
fragment iii is: 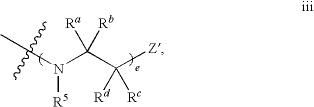
wherein, e is 0 or 1, and Z′, R^{a}, R^{ip}, R^{c}, R^{d} and R⁵ are as described above;
and fragment iv is: 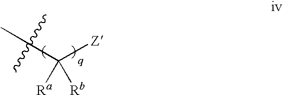
wherein Z′, R^{a} and R^{b} are as described above, and q is an integer from 1 to 10;
or R²³ and R²⁴ taken together form -Ch,
wherein, in each instance, “-Ch” is a tetradentate chelating ligand capable of complexing with a metal to form a metal chelate;
provided that one of R²² and R²³ is other than hydrogen.

27. The compound of claim 26, wherein said -Ch is a N₂S₂ type ligand.

28. A radiometal complex of a compound of claim 26.

29. A pharmaceutical composition comprising a compound of claim 1 or 26 and a pharmaceutically acceptable excipient.

30. A diagnostic composition for imaging amyloid deposits, comprising a radiolabeled compound of claim 1 or 26.

31. A method of imaging amyloid deposits, comprising:
a. introducing into a mammal a detectable quantity of a diagnostic composition of claim 30;
b. allowing sufficient time for the labeled compound to be associated with amyloid deposits; and
c. detecting the labeled compound associated with one or more amyloid deposits.

32. A method of inhibiting amyloid plaque aggregation in a mammal, comprising administering a composition of claim 29 in an amount effective to inhibit amyloid plaque aggregation.

33. The compound of claim 1, having the formula:

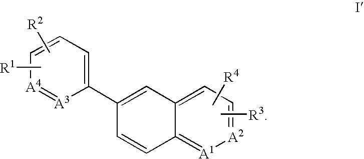

34. The compound of claim 26, having the following formula: [chemical structure]
